SEQUENCE LISTING (59 sequences)

SEQ ID NO: 1 - protein, 736 aa, synthetic construct
  REGION 1..736: note = Adeno-associated virus 9.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - protein, 736 aa, synthetic construct
  REGION 1..736: note = AAV isolate.
MTADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKQLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3 - protein, 736 aa, synthetic construct
  REGION 1..736: note = AAV isolate.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTGGNFHPSPLMGGFGMKHPPPQILIKNTPVPADPPTAFNKDKLNSFITQYSTGQVSVEIEWELQKENSKRWNPEIQYTSNYYKSNNVEFAVNTGGVYSEPRPIGTRYLTRNL

SEQ ID NO: 4 - protein, 736 aa, synthetic construct
  REGION 1..736: note = AAV isolate.
MAADGYLPDWLEDNLSEGIREWWALKPGAPQPKANQQHQDNARGLVLPGYKYLGPGNGLDKGEPINAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTYGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 5 - protein, 736 aa, synthetic construct
  REGION 1..736: note = AAV isolate.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIDKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 6 - protein, 736 aa, synthetic construct
  REGION 1..736: note = AAV isolate.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVLQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGSSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 7 - protein, 736 aa, synthetic construct
  REGION 1..736: note = AAV isolate.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQARAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 8 - protein, 736 aa, synthetic construct
  REGION 1..736: note = AAV isolate.
MAADGYLPDWLEDNLSEGIREWWALKPGAPQPKANQQHQDNARGLVLPGYKYLGPGNGLDKGEPVNAVDAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 9 - protein, 736 aa, synthetic construct
  REGION 1..736: note = AAV isolate.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPREPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 736 aa, synthetic construct
  REGION 1..736: note = AAV isolate.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASCGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - protein, 736 aa, synthetic construct
  REGION 1..736: note = AAV isolate.
MAADGYLPDWLEDNLSEGIREWWALKPGAPQPKANQQHQDNARGLVLPGYKYLGPGNGLDKGEPVNAADAAALEHDRAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKKNSKRWNPEIQYTSNYYKSNNVEFAVNTEGVYSEPRPIGTRYLTRNL

SEQ ID NO: 12 - protein, 736 aa, synthetic construct
  REGION 1..736: note = AAV isolate.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPHDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFNVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSMEIEWELQKENSKRWNPEIQYTSNYYKSNNVEFAVNTEGVYSEPRPIGTRYLTRNL

SEQ ID NO: 13 - protein, 736 aa, synthetic construct
  REGION 1..736: note = AAV isolate.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 14 - DNA, 198 bp, synthetic construct
  misc_feature 1..198: note = Synthetic polynucleotide.
  source 1..198: mol_type = other DNA
gatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 15 - protein, 736 aa, synthetic construct
  REGION 1..736: note = AAV isolate.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWERQKENSKRWNPEIQYTSNYYKSNNVEFAVNTEGVYSEPRPIGTRYLTRNL

SEQ ID NO: 16 - protein, 736 aa, synthetic construct
  REGION 1..736: note = AAV isolate.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFADSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 17 - protein, 736 aa, synthetic construct
  REGION 1..736: note = AAV isolate.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEIAWPRASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYCKSNNVEFAVNTEGVYSEPRPIGTRYLTRNL

SEQ ID NO: 18 - DNA, 365 bp, synthetic construct
  misc_feature 1..365: note = Synthetic polynucleotide.
  source 1..365: mol_type = other DNA
ctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatg

SEQ ID NO: 19 - DNA, 380 bp, synthetic construct
  misc_feature 1..380: note = Synthetic polynucleotide.
  source 1..380: mol_type = other DNA
ggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatg

SEQ ID NO: 20 - DNA, 479 bp, synthetic construct
  misc_feature 1..479: note = Synthetic polynucleotide.
  source 1..479: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggag

SEQ ID NO: 21 - DNA, 1246 bp, synthetic construct
  misc_feature 1..1246: note = Synthetic polynucleotide.
  source 1..1246: mol_type = other DNA
tcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcgg

SEQ ID NO: 22 - DNA, 953 bp, synthetic construct
  misc_feature 1..953: note = Synthetic polynucleotide.
  source 1..953: mol_type = other DNA
aattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaag

SEQ ID NO: 23 - DNA, 1168 bp, synthetic construct
  misc_feature 1..1168: note = Synthetic polynucleotide.
  source 1..1168: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctccagtacgtgattcttgatcccgagctggagccaggggcgggccttgcgctttaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaggatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctccagggggctcaaaatggaggacgcggcgctcgggagagcgggcgggtgagtcacccacacaaaggaaaggggcctttccgtcctcagccgtcgcttcatgtgactccacggagtaccgggcgccgtccaggcacctcgattagttctggagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 24 - DNA, 92 bp, synthetic construct
  misc_feature 1..92: note = Synthetic polynucleotide.
  source 1..92: mol_type = other DNA
aagaggtaagggtttaagggatggttggttggtggggtattaatgtttaattacctggagcacctgcctgaaatcactttttttcaggttgg

SEQ ID NO: 25 - DNA, 79 bp, synthetic construct
  misc_feature 1..79: note = Synthetic polynucleotide.
  source 1..79: mol_type = other DNA
aagaggtaagggtttaagggatggttggttggtggggtattaatgtttaattacctggagcacctgcctgaaatcactt

SEQ ID NO: 26 - DNA, 95 bp, synthetic construct
  misc_feature 1..95: note = Synthetic polynucleotide.
  source 1..95: mol_type = other DNA
cctctgctaaccatgttcatgccttcttctttttcctacagctcctgggcaacgtgctggttattgtgctgtctcatcattttggcaaagaattc

SEQ ID NO: 27 - DNA, 90 bp, synthetic construct
  misc_feature 1..90: note = Synthetic polynucleotide.
  source 1..90: mol_type = other DNA
cctctgctaaccatgttcatgccttcttctttttcctacagctcctgggcaacgtgctggttattgtgctgtctcatcattttggcaaag

SEQ ID NO: 28 - DNA, 918 bp, synthetic construct
  misc_feature 1..918: note = Synthetic polynucleotide.
  source 1..918: mol_type = other DNA
ggcatcctaaaaaatattcagtggaaacgtaaaaacattaaagactgattaaacatcgcagcatgacacagatttagcaactgagcataaataatttgactcggatactgctccaaaatccgaagaggaccaatttcttccaggaggacaactacctcgtcctctgcagacccctctcctcggcagctgaaggagtgtggccaatctgcctccacctccccgcggaccccctactctcaggacctcctgcagcaccccaaactggaagtggccgctgcagacccaaggacgaggggcacgcgggagccggcagccctagtggagcggttggagatgttgaggtgggagggtcacccaggtggggtgaggctggggtaggtagcggagtgaacggcttccgaagctctgggccgcccccaggttggactaagcaggcgctctgtcttcgcccccgcccagggtgggcgtctcctgaggactccccgccacacctgacccgagaccgcgcgcccagcctagaacgcttccccgacccagcgtagggccgccgcgactggcgggcgagggtcggcgggaggcctggcgaacccgggggcgggaccaggcgggcaaggcccggctgccgcagcgccgctctgcgcgaggcggctccgccgcggcggagggatacggcgcaccatatatatatcgcggggcgcagactcgcgctccggcagtggtgctgggagtgtcgtggacgccgtgccgttactcgtagtcaggcggcggcgcaggcggcggcggcggcatagcgcacagcgcgccttagcagcagcagcagcagcagcggcatcggaggtacccccgccgtcgcagcccccgcgctggtgcagccaccctcgctccctctgctcttcctcccttcgctcgcaccaagag

SEQ ID NO: 29 - DNA, 192 bp, synthetic construct
  misc_feature 1..192: note = Synthetic polynucleotide.
  source 1..192: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 30 - DNA, 205 bp, synthetic construct
  misc_feature 1..205: note = Synthetic polynucleotide.
  source 1..205: mol_type = other DNA
aatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagg

SEQ ID NO: 31 - DNA, 913 bp, synthetic construct
  misc_feature 1..913: note = Synthetic polynucleotide.
  source 1..913: mol_type = other DNA
tagggaggtcctgcacagaaggggaggagggggcagcagctgtctgaccactgttggtcttgcaacttgtgtccccaggttaatttttaaaaagcagtcaaaagtccaagtggcccttggcagcatttactctctctgtttgctctggttaataatctcaggagcacaaacattcctggaggcaggagaagaaatcaacatcctggacttatcctctgggcctctccccacccccaggattgtaactgaaatgcttcactggtgctccttttgttttaaggcattggatcttcatagctactgatcgtgcccaagcacacagtatctgcagcaaccacttaggcctccaggaatgtggtgaccattgaccctaattcattccccttcatggatcctatgtaaccatcctccaaaaagagctttcgcaaactcaaataaacacaggaaaggaagaccttcttatctttgagagtatatgtttagccctatagctctaacccactctgatctcccagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggac

SEQ ID NO: 32 - DNA, 423 bp, synthetic construct
  misc_feature 1..423: note = Synthetic polynucleotide.
  source 1..423: mol_type = other DNA
gctctaacccactctgatctcccagggcggcagtaagtcttcagcatcaggcattttggggtgactcagtaaatggtagatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggac

SEQ ID NO: 33 - DNA, 592 bp, synthetic construct
  misc_feature 1..592: note = Synthetic polynucleotide.
  source 1..592: mol_type = other DNA
gtaaattttatggaatgtgaatcataattcaatttttcaacatgcgttaggagggacatttcaaactcttttttaccctagactttcctaccatcacccagagtatccagccaggaggggaggggctagagacaccagaagtttagcagggaggagggcgtagggattcggggaatgaagggatgggattcagactagggccaggacccagggatggagagaaagagatgagagtggtttgggggcttggtgacttagagaacagagctgcaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 34 - DNA, 72 bp, synthetic construct
  misc_feature 1..72: note = Synthetic polynucleotide.
  source 1..72: mol_type = other DNA
gggggaggctgctggtgaatattaaccaaggtcaccccagttatcggaggagcaaacaggggctaagtccac

SEQ ID NO: 35 - DNA, 170 bp, synthetic construct
  misc_feature 1..170: note = Synthetic polynucleotide.
  source 1..170: mol_type = other DNA
cgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatca

SEQ ID NO: 36 - DNA, 1873 bp, synthetic construct
  misc_feature 1..1873: note = Synthetic polynucleotide.
  source 1..1873: mol_type = other DNA
gatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaatt

SEQ ID NO: 37 - DNA, 1061 bp, synthetic construct
  misc_feature 1..1061: note = Synthetic polynucleotide.
  source 1..1061: mol_type = other DNA
tagggaggtcctgcacgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactaaaacaggtaagtccggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgatgcctctactaaccatgttcatgttttctttttttttctacaggtcctgggtgacgaacag

SEQ ID NO: 38 - DNA, 398 bp, synthetic construct
  misc_feature 1..398: note = Synthetic polynucleotide.
  source 1..398: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagg

SEQ ID NO: 39 - DNA, 145 bp, synthetic construct
  misc_feature 1..145: note = Synthetic polynucleotide.
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 40 - DNA, 145 bp, synthetic construct
  misc_feature 1..145: note = Synthetic polynucleotide.
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 41 - DNA, 106 bp, synthetic construct
  misc_feature 1..106: note = Synthetic polynucleotide.
  source 1..106: mol_type = other DNA
ctgcgcgctcgctcgctcactgaggccgcccgggcaaagcccgggcgtcgggcgacctttggtcgcccggcctcagtgagcgagcgagcgcgcagagagggagtgg

SEQ ID NO: 42 - DNA, 167 bp, synthetic construct
  misc_feature 1..167: note = Synthetic polynucleotide.
  source 1..167: mol_type = other DNA
ctctcccccctgtcgcgttcgctcgctcgctggctcgtttgggggggtggcagctcaaagagctgccagacgacggccctctggccgtcgcccccccaaacgagccagcgagcgagcgaacgcgacaggggggagagtgccacactctcaagcaagggggttttgta

SEQ ID NO: 43 - DNA, 167 bp, synthetic construct
  misc_feature 1..167: note = Synthetic polynucleotide.
  source 1..167: mol_type = other DNA
tacaaaacctccttgcttgagagtgtggcactctcccccctgtcgcgttcgctcgctcgctggctcgtttgggggggtggcagctcaaagagctgccagacgacggccctctggccgtcgcccccccaaacgagccagcgagcgagcgaacgcgacaggggggagag

SEQ ID NO: 44 - DNA, 143 bp, synthetic construct
  misc_feature 1..143: note = Synthetic polynucleotide.
  source 1..143: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 45 - DNA, 46 bp, synthetic construct
  misc_feature 1..46: note = Synthetic polynucleotide.
  source 1..46: mol_type = other DNA
ggaggggtggagtcgtgacgtgaattacgtcatagggttagggagg

SEQ ID NO: 46 - DNA, 37 bp, synthetic construct
  misc_feature 1..37: note = Synthetic polynucleotide.
  source 1..37: mol_type = other DNA
gtagataagtagcatggcgggttaatcattaactaca

SEQ ID NO: 47 - DNA, 122 bp, synthetic construct
  misc_feature 1..122: note = Synthetic polynucleotide.
  source 1..122: mol_type = other DNA
aacttgtttattgcagcttataatggttacaaataaagcaatagcatcacaaatttcacaaataaagcatttttttcactgcattctagttgtggtttgtccaaactcatcaatgtatctta

SEQ ID NO: 48 - DNA, 133 bp, synthetic construct
  misc_feature 1..133: note = Synthetic polynucleotide.
  source 1..133: mol_type = other DNA
tgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 49 - DNA, 171 bp, synthetic construct
  misc_feature 1..171: note = Synthetic polynucleotide.
  source 1..171: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagg

SEQ ID NO: 50 - DNA, 4075 bp, synthetic construct
  misc_feature 1..4075: note = Synthetic polynucleotide.
  source 1..4075: mol_type = other DNA
ccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctcgaggaacgttttattttcccagagacaggctcagggacttctgggtgtagtggctgtgcagggcttcgtggagcacgctgcagctaaacacgttgccctcttgccatcggctcttgtccacggtcagccgtgaatacaggaagaagctgccatcgctgtccagcactggaggggtcgtcttgtagttgttttcaggctgtccgttgctctcccattcgacggcgatgtcgctggggtagaagcccttcaccaggcaggtcagagacacttggttctttgtcatttcttcttgagaaggaggcagtgtatagacttggggctctctgggctggcccttggcctttgagatggtcttctcgatgctgctaggcaggcccttgttagacaccttgcacttgtactctttgccgttcagccagtcctggtgcagcactgtcagcacggacaccacccggtaggtgctgttaaactgttcctcgcggggtttggtcttggcattatgaacttccactccgtcaacgtaccaattaaactgcacctcaggatcctcctgggacacatccaccaccacacatgtcacttcaggggtccggctgatcatcagggtgtctttgggctttggtgggaacaggaaaacagaagggccggcgactggtggagctggacatggtgggcattccacgcagcactttctctcaacggtcttatccactttggtgttgctaggcttgtggtccacattacaggtgtaggtctgtgtgccgaagttggagctaggaacggtgaccacgctactcagagaatacaggccagagctctgcagcacggctgggaaggtgtgcacgccgcttgtcagggcgccgctgttccagctcactgtcacgggttcggggaagtagtctttgaccagacaacccagggcggctgtgctttcagatgtgcttctgctgcatggggccagtgggaacacgctgggtcccttagtactggcgctgctgacggtgacaagggtgccttggccccacacgtcgaagtaccagttaggggatgagccaaaaaagtatctggcgcagtagtacacagctgtgtcctcgcttctcaggctgctcagttccatgtacacggtgcttgtggaggtatctctggtcatggtcactctatccttgaagttctctgtgtactcggtgtggccagatcctggcaggatctcgcccatccattccaggccttggccaggggcctgccgcacccactggatccagtagttgctgaagatgtggccgctggccttgcaggacaccttcacgctagctccgggctttttcacttcggctccgctttgaaccagctgcacttggctgtgcacgcctgtggcggtggccaccaggaacaggatgatgcaggaccagcccatggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggccgcggcacgtgcttaaggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatctccggagggggaggctgctggtgaatattaaccaaggtcaccccagttatcggaggagcaaacaggggctaagtccacctcgagccatggcgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaa

SEQ ID NO: 51 - DNA, 3533 bp, synthetic construct
  misc_feature 1..3533: note = Synthetic polynucleotide.
  source 1..3533: mol_type = other DNA
gcttcaggagcagttgtgcgaatagctggagaacaccaggctggatttaaacccagatcgctcttacatttgctctttacctgctgtgctcagcgttcacgtgccctctagctgtagttttctgaagtcagcgcacagcaaggcagtgtgcttagaggttaacagaagggaaaacaacaacaacaaaaatctaaatgagaatcctgactgtttcagctgggggtaaggggggcggattattcatataattgttataccagacggtcgcaggcttagtccaattgcagagaactcgcttcccaggcttctgagagtcccggaagtgcctaaacctgtctaatcgacggggcttgggtggcccgtcgctccctggcttcttccctttacccagggcgggcagcgaagtggtgcctcctgcgtcccccacaccctccctcagcccctcccctccggcccgtcctgggcaggtgacctggagcatccggcaggctgccctggcctcctgcgtcaggacaacgcccacgaggggcgttactgtgcggagatgcaccacgcaagagacaccctttgtaactctcttctcctccctagtgcgaggttaaaaccttcagccccacgtgctgtttgcaaacctgcctgtacctgaggccctaaaaagccagagacctcactcccggggagccagcatgtccactgcggtcctggaaaacccaggcttgggcaggaaactctctgactttggacaggtgagccacggcagcctgagctgctcagttaggggaatttgggcctccagagaaagagatctgaagactgctggtgcttcctggtttcataagctcagtaagaagtctgaattcgttggaagctgatgagaatatccaggaagtcaacagacaaatgtcctcaacaattgtttctaagtaggagaacatctgtcctcggtggctttcacaggaaaagcttctgacctcttctcttcctcccacagggcggtaccagatctggcagcggagagggcagaggaagtcttctaacatgcggtgacgtggaggagaatcccggcccttccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagcgtgcggtacgacccttatacccagaggatcgaggtgctggataatacacagcagctgaagatcctggctgactcaatcaatagcgaaatcggaatcctgtgctccgccctgcagaaaatcaaatgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaactgggatgggatgtggaatccttctagatttcttttgtaatatttataaagtgctctcagcaaggtatcaaaatggcaaaattgtgagtaactatcctcctttcattttgggaagaagatgaggcatgaagagaattcagacagaaacttactcagaccaggggaggcagaaactaagcagagaggaaaatgaccaagagttagccctgggcatggaatgtgaaagaaccctaaacgtgacttggaaataatgcccaaggtatattccattctccgggatttgttggcattttcttgaggtgaagaattgcagaatacattctttaatgtgacctacatatttacccatgggaggaagtctgctcctggactcttgagattcagtcataaagcccaggccagggaaataatgtaagtctgcaggcccctgtcatcagtaggattagggagaagagttctcagtagaaaacagggaggctggagagaaaagaatggttaatgttaacgttaatataactagaaagactgcagaacttaggactgatttttatttgaatccttaaaaaaaaaatttcttatgaaaatagtacatggctcttaggagacagaacttattgtacagaggaacagcgtgagagtcagagtgatcccagaacaggtcctggctccatcctgcacatagttttggtgctgctggcaatacggtccccacaactgtgggaaggggttaggggcagggatctcatcaggaaagcataggggtttaaagttctttatagagcacttagaagattgagaatccacaaattatattaataacaaacaaagtagtgtcgtgttatatagtaaatgtgaatttgcagacacatttagggaaaagttataattaaaaaaataggctgtatatata

SEQ ID NO: 52 - DNA, 4061 bp, synthetic construct
  misc_feature 1..4061: note = Synthetic polynucleotide.
  source 1..4061: mol_type = other DNA
tgcggccgcttcaggagcagttgtgcgaatagctggagaacaccaggctggatttaaacccagatcgctcttacatttgctctttacctgctgtgctcagcgttcacgtgccctctagctgtagttttctgaagtcagcgcacagcaaggcagtgtgcttagaggttaacagaagggaaaacaacaacaacaaaaatctaaatgagaatcctgactgtttcagctgggggtaaggggggcggattattcatataattgttataccagacggtcgcaggcttagtccaattgcagagaactcgcttcccaggcttctgagagtcccggaagtgcctaaacctgtctaatcgacggggcttgggtggcccgtcgctccctggcttcttccctttacccagggcgggcagcgaagtggtgcctcctgcgtcccccacaccctccctcagcccctcccctccggcccgtcctgggcaggtgacctggagcatccggcaggctgccctggcctcctgcgtcaggacaacgcccacgaggggcgttactgtgcggagatgcaccacgcaagagacaccctttgtaactctcttctcctccctagtgcgaggttaaaaccttcagccccacgtgctgtttgcaaacctgcctgtacctgaggccctaaaaagccagagacctcactcccggggagccagcatgtccactgcggtcctggaaaacccaggcttgggcaggaaactctctgactttggacaggtgagccacggcagcctgagctgctcagttaggggaatttgggcctccagagaaagagatctgaagactgctggtgcttcctggtttcataagctcagtaagaagtctgaattcgttggaagctgatgagaatatccaggaagtcaacagacaaatgtcctcaacaattgtttctaagtaggagaacatctgtcctcggtggctttcacaggaaccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaccgccaccatgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagtgtgcgttacgacccttatacccagaggatcgaggtgctggataatacacagcagctgaagatcctggctgactcaatcaatagcgaaatcggaatcctgtgctccgccctgcagaaaatcaaatgagaattcaaggcctctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagctttacgtacgatcgtcgactgggatgggatgtggaatccttctagatttcttttgtaatatttataaagtgctctcagcaaggtatcaaaatggcaaaattgtgagtaactatcctcctttcattttgggaagaagatgaggcatgaagagaattcagacagaaacttactcagaccaggggaggcagaaactaagcagagaggaaaatgaccaagagttagccctgggcatggaatgtgaaagaaccctaaacgtgacttggaaataatgcccaaggtatattccattctccgggatttgttggcattttcttgaggtgaagaattgcagaatacattctttaatgtgacctacatatttacccatgggaggaagtctgctcctggactcttgagattcagtcataaagcccaggccagggaaataatgtaagtctgcaggcccctgtcatcagtaggattagggagaagagttctcagtagaaaacagggaggctggagagaaaagaatggttaatgttaacgttaatataactagaaagactgcagaacttaggactgatttttatttgaatccttaaaaaaaaaatttcttatgaaaatagtacatggctcttaggagacagaacttattgtacagaggaacagcgtgagagtcagagtgatcccagaacaggtcctggctccatcctgcacatagttttggtgctgctggcaatacggtccccacaactgtgggaaggggttaggggcagggatctcatcaggaaagcataggggtttaaagttctttatagagcacttagaagattgagaatccacaaattatattaataacaaacaaagtagtgtcgtgttatatagtaaatgtgaatttgcagacacatttagggaaaagttataattaaaaaaataggctgtatatatacctgcagg

SEQ ID NO: 53 - DNA, 2141 bp, synthetic construct
  misc_feature 1..2141: note = Synthetic polynucleotide.
  source 1..2141: mol_type = other DNA
gcggccgcaactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtgccaccatgcccccacccaggaccggaagaggcctgctgtggctgggcctggtgctctcttccgtgtgcgtggccctgggaagcgaaacccaggccaacagcacaaccgacgccctgaatgtgctgctgatcattgtggacgatctgagaccctccctgggctgttacggcgacaaactggtgcggtccccaaacatcgaccagctggcctcccactccctgctgttccagaacgccttcgcccagcaggccgtgtgtgcccccagcagggtgagcttcctgaccggcagaagacctgacaccaccaggctgtacgactttaacagctactggcgggtgcacgccggcaatttcagcaccattcctcagtacttcaaggagaatggctacgtgacaatgtccgtgggcaaggtgtttcatcccggcattagctccaaccacaccgacgatagcccatactcctggtccttccccccctaccatccctccagcgagaagtacgagaacaccaaaacctgcagaggccctgacggagagctgcacgccaacctgctgtgccctgtggatgtcctggatgtgcccgaaggtaagggtttaagggatggttggttggtggggtattaatgtttaattacctggagcacctgcctgaaatcactttttttcaggcaccctgccagacaagcagtccacagagcaggccatccagctgctggagaagatgaagacaagcgccagccccttctttctggccgtgggataccacaagcctcacattccattccggtacccaaaagagttccagaagctgtaccctctggaaaacatcaccctggcccctgaccccgaggtgccagacgggctgcctcctgtggcctacaatccctggatggacatcagacagcgggaggatgtgcaggccctgaatatttccgtgccctatgggcccatccctgtggactttcagcggaaaatcagacagtcttactttgccagcgtgtcctacctggacacccaggtgggccgcctgctctcagccctggacgacctgcagctggccaattccaccatcatcgccttcaccagcgatcacggctgggccctgggcgagcacggggagtgggccaaatacagcaacttcgatgtggccacccacgtgcctctgattttttatgtgcccggccggacagccagcctgcccgaggccggggagaagctctttccttacctggaccctttcgactctgccagccagctgatggaacctggcagacagagcatggacctggtggagctggtgagcctcttccccactctggccggcctggctggcctgcaggtgccaccaagatgcccagtgccttctttccacgtggagctgtgtagagagggaaagaacctgctgaagcacttcagatttagagatctggaggaggatccctacctgccaggcaacccaagggagctgatcgcctacagccagtatcccagaccctctgatatcccccagtggaacagcgataagccctccctgaaagacatcaagattatgggctactccatcaggaccattgactaccggtacacagtgtgggtgggcttcaaccccgatgagtttctggccaacttcagcgacatccacgccggcgagctgtattttgtggactccgaccccctgcaggaccacaacatgtacaacgactcccagggcggcgacctgttccagctgctgatgccctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaacctgcaggtctagatacgta

SEQ ID NO: 54 - DNA, 2042 bp, synthetic construct
  misc_feature 1..2042: note = Synthetic polynucleotide.
  source 1..2042: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaccgccaccatgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagcgtgcggtacgacccttatacccagaggatcgaggtgctggataatacacagcagctgaagatcctggctgactcaatcaatagcgaaatcggaatcctgtgctccgccctgcagaaaatcaaatgaatgctttatttgtgaaatttgtgatgctattgctttatttgtaaccattataagctgcaataaacaagttaacaacaacaattgcattcattttatgtttcaggttcagggggaggtgtgggaggttttttaaa

SEQ ID NO: 55 - DNA, 3939 bp, synthetic construct
  misc_feature 1..3939: note = Synthetic polynucleotide.
  source 1..3939: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcttcaggagcagttgtgcgaatagctggagaacaccaggctggatttaaacccagatcgctcttacatttgctctttacctgctgtgctcagcgttcacgtgccctctagctgtagttttctgaagtcagcgcacagcaaggcagtgtgcttagaggttaacagaagggaaaacaacaacaacaaaaatctaaatgagaatcctgactgtttcagctgggggtaaggggggcggattattcatataattgttataccagacggtcgcaggcttagtccaattgcagagaactcgcttcccaggcttctgagagtcccggaagtgcctaaacctgtctaatcgacggggcttgggtggcccgtcgctccctggcttcttccctttacccagggcgggcagcgaagtggtgcctcctgcgtcccccacaccctccctcagcccctcccctccggcccgtcctgggcaggtgacctggagcatccggcaggctgccctggcctcctgcgtcaggacaacgcccacgaggggcgttactgtgcggagatgcaccacgcaagagacaccctttgtaactctcttctcctccctagtgcgaggttaaaaccttcagccccacgtgctgtttgcaaacctgcctgtacctgaggccctaaaaagccagagacctcactcccggggagccagcatgtccactgcggtcctggaaaacccaggcttgggcaggaaactctctgactttggacaggtgagccacggcagcctgagctgctcagttaggggaatttgggcctccagagaaagagatctgaagactgctggtgcttcctggtttcataagctcagtaagaagtctgaattcgttggaagctgatgagaatatccaggaagtcaacagacaaatgtcctcaacaattgtttctaagtaggagaacatctgtcctcggtggctttcacaggaaaagcttctgacctcttctcttcctcccacagggcggtaccagatctggcagcggagagggcagaggaagtcttctaacatgcggtgacgtggaggagaatcccggcccttccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagcgtgcggtacgacccttatacccagaggatcgaggtgctggataatacacagcagctgaagatcctggctgactcaatcaatagcgaaatcggaatcctgtgctccgccctgcagaaaatcaaatgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaactgggatgggatgtggaatccttctagatttcttttgtaatatttataaagtgctctcagcaaggtatcaaaatggcaaaattgtgagtaactatcctcctttcattttgggaagaagatgaggcatgaagagaattcagacagaaacttactcagaccaggggaggcagaaactaagcagagaggaaaatgaccaagagttagccctgggcatggaatgtgaaagaaccctaaacgtgacttggaaataatgcccaaggtatattccattctccgggatttgttggcattttcttgaggtgaagaattgcagaatacattctttaatgtgacctacatatttacccatgggaggaagtctgctcctggactcttgagattcagtcataaagcccaggccagggaaataatgtaagtctgcaggcccctgtcatcagtaggattagggagaagagttctcagtagaaaacagggaggctggagagaaaagaatggttaatgttaacgttaatataactagaaagactgcagaacttaggactgatttttatttgaatccttaaaaaaaaaatttcttatgaaaatagtacatggctcttaggagacagaacttattgtacagaggaacagcgtgagagtcagagtgatcccagaacaggtcctggctccatcctgcacatagttttggtgctgctggcaatacggtccccacaactgtgggaaggggttaggggcagggatctcatcaggaaagcataggggtttaaagttctttatagagcacttagaagattgagaatccacaaattatattaataacaaacaaagtagtgtcgtgttatatagtaaatgtgaatttgcagacacatttagggaaaagttataattaaaaaaataggctgtatatatacctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 56 - DNA, 2356 bp, synthetic construct
  misc_feature 1..2356: note = Synthetic polynucleotide.
  source 1..2356: mol_type = other DNA
ctgcgcgctcgctcgctcactgaggccgcccgggcaaagcccgggcgtcgggcgacctttggtcgcccggcctcagtgagcgagcgagcgcgcagagagggagtggaattcacgcgtggatctgaattcaattcacgcgtggtacctccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaccgccaccatgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagcgtgcggtacgacccttatacccagaggatcgaggtgctggataatacacagcagctgaagatcctggctgactcaatcaatagcgaaatcggaatcctgtgctccgccctgcagaaaatcaaatgaatgctttatttgtgaaatttgtgatgctattgctttatttgtaaccattataagctgcaataaacaagttaacaacaacaattgcattcattttatgtttcaggttcagggggaggtgtgggaggttttttaaagcatgctggggagagatcgatctgaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggcc

SEQ ID NO: 57 - DNA, 4452 bp, synthetic construct
  misc_feature 1..4452: note = Synthetic polynucleotide.
  source 1..4452: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcttcaggagcagttgtgcgaatagctggagaacaccaggctggatttaaacccagatcgctcttacatttgctctttacctgctgtgctcagcgttcacgtgccctctagctgtagttttctgaagtcagcgcacagcaaggcagtgtgcttagaggttaacagaagggaaaacaacaacaacaaaaatctaaatgagaatcctgactgtttcagctgggggtaaggggggcggattattcatataattgttataccagacggtcgcaggcttagtccaattgcagagaactcgcttcccaggcttctgagagtcccggaagtgcctaaacctgtctaatcgacggggcttgggtggcccgtcgctccctggcttcttccctttacccagggcgggcagcgaagtggtgcctcctgcgtcccccacaccctccctcagcccctcccctccggcccgtcctgggcaggtgacctggagcatccggcaggctgccctggcctcctgcgtcaggacaacgcccacgaggggcgttactgtgcggagatgcaccacgcaagagacaccctttgtaactctcttctcctccctagtgcgaggttaaaaccttcagccccacgtgctgtttgcaaacctgcctgtacctgaggccctaaaaagccagagacctcactcccggggagccagcatgtccactgcggtcctggaaaacccaggcttgggcaggaaactctctgactttggacaggtgagccacggcagcctgagctgctcagttaggggaatttgggcctccagagaaagagatctgaagactgctggtgcttcctggtttcataagctcagtaagaagtctgaattcgttggaagctgatgagaatatccaggaagtcaacagacaaatgtcctcaacaattgtttctaagtaggagaacatctgtcctcggtggctttcacaggaaccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtgaatcctctaaggtaaatataaaatttttaagtgtataatgtgttaaactactgattctaattgtttctctcttttagattccaacctttggaactgaccgccaccatgtccaccgctgtgctggagaaccctgggctggggaggaaactgtcagacttcgggcaggagacttcatacattgaggataactgtaaccagaatggcgccatctctctgatcttcagcctgaaggaggaagtgggcgccctggcaaaggtgctgcgcctgtttgaggagaacgacgtgaatctgacccacatcgagtcccggccttctagactgaagaaggacgagtacgagttctttacccacctggataagcggtccctgccagccctgacaaacatcatcaagatcctgaggcacgacatcggagcaaccgtgcacgagctgtctcgggacaagaagaaggataccgtgccctggttccctcggacaatccaggagctggatagatttgccaaccagatcctgtcttacggagcagagctggacgcagatcaccctggcttcaaggacccagtgtatcgggcccggagaaagcagtttgccgatatcgcctacaattataggcacggacagccaatccctcgcgtggagtatatggaggaggagaagaagacctggggcacagtgttcaagaccctgaagagcctgtacaagacacacgcctgctacgagtataaccacatcttccccctgctggagaagtattgtggctttcacgaggacaatatccctcagctggaggacgtgagccagttcctgcagacctgcacaggctttaggctgaggccagtggcaggactgctgagctcccgggacttcctgggaggactggccttcagagtgtttcactgcacccagtacatcaggcacggctccaagccaatgtatacaccagagcccgacatctgtcacgagctgctgggccacgtgcccctgtttagcgatagatccttcgcccagttttcccaggagatcggactggcatctctgggagcacctgacgagtacatcgagaagctggccaccatctattggttcacagtggagtttggcctgtgcaagcagggcgatagcatcaaggcctacggagcaggactgctgtctagcttcggcgagctgcagtattgtctgtccgagaagccaaagctgctgcccctggagctggagaagaccgccatccagaactacaccgtgacagagttccagcccctgtactatgtggccgagtcttttaacgatgccaaggagaaggtgagaaatttcgccgccacaatccctaggcccttcagtgtgcgttacgacccttatacccagaggatcgaggtgctggataatacacagcagctgaagatcctggctgactcaatcaatagcgaaatcggaatcctgtgctccgccctgcagaaaatcaaatgagaattcaaggcctctcgagcctctagaactatagtgagtcgtattacgtagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagctttacgtacgatcgtcgactgggatgggatgtggaatccttctagatttcttttgtaatatttataaagtgctctcagcaaggtatcaaaatggcaaaattgtgagtaactatcctcctttcattttgggaagaagatgaggcatgaagagaattcagacagaaacttactcagaccaggggaggcagaaactaagcagagaggaaaatgaccaagagttagccctgggcatggaatgtgaaagaaccctaaacgtgacttggaaataatgcccaaggtatattccattctccgggatttgttggcattttcttgaggtgaagaattgcagaatacattctttaatgtgacctacatatttacccatgggaggaagtctgctcctggactcttgagattcagtcataaagcccaggccagggaaataatgtaagtctgcaggcccctgtcatcagtaggattagggagaagagttctcagtagaaaacagggaggctggagagaaaagaatggttaatgttaacgttaatataactagaaagactgcagaacttaggactgatttttatttgaatccttaaaaaaaaaatttcttatgaaaatagtacatggctcttaggagacagaacttattgtacagaggaacagcgtgagagtcagagtgatcccagaacaggtcctggctccatcctgcacatagttttggtgctgctggcaatacggtccccacaactgtgggaaggggttaggggcagggatctcatcaggaaagcataggggtttaaagttctttatagagcacttagaagattgagaatccacaaattatattaataacaaacaaagtagtgtcgtgttatatagtaaatgtgaatttgcagacacatttagggaaaagttataattaaaaaaataggctgtatatatacctgcaggtctagatacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 58 - DNA, 2521 bp, synthetic construct
  misc_feature 1..2521: note = Synthetic polynucleotide.
  source 1..2521: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcatatgcggccgcaactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtgccaccatgcccccacccaggaccggaagaggcctgctgtggctgggcctggtgctctcttccgtgtgcgtggccctgggaagcgaaacccaggccaacagcacaaccgacgccctgaatgtgctgctgatcattgtggacgatctgagaccctccctgggctgttacggcgacaaactggtgcggtccccaaacatcgaccagctggcctcccactccctgctgttccagaacgccttcgcccagcaggccgtgtgtgcccccagcagggtgagcttcctgaccggcagaagacctgacaccaccaggctgtacgactttaacagctactggcgggtgcacgccggcaatttcagcaccattcctcagtacttcaaggagaatggctacgtgacaatgtccgtgggcaaggtgtttcatcccggcattagctccaaccacaccgacgatagcccatactcctggtccttccccccctaccatccctccagcgagaagtacgagaacaccaaaacctgcagaggccctgacggagagctgcacgccaacctgctgtgccctgtggatgtcctggatgtgcccgaaggtaagggtttaagggatggttggttggtggggtattaatgtttaattacctggagcacctgcctgaaatcactttttttcaggcaccctgccagacaagcagtccacagagcaggccatccagctgctggagaagatgaagacaagcgccagccccttctttctggccgtgggataccacaagcctcacattccattccggtacccaaaagagttccagaagctgtaccctctggaaaacatcaccctggcccctgaccccgaggtgccagacgggctgcctcctgtggcctacaatccctggatggacatcagacagcgggaggatgtgcaggccctgaatatttccgtgccctatgggcccatccctgtggactttcagcggaaaatcagacagtcttactttgccagcgtgtcctacctggacacccaggtgggccgcctgctctcagccctggacgacctgcagctggccaattccaccatcatcgccttcaccagcgatcacggctgggccctgggcgagcacggggagtgggccaaatacagcaacttcgatgtggccacccacgtgcctctgattttttatgtgcccggccggacagccagcctgcccgaggccggggagaagctctttccttacctggaccctttcgactctgccagccagctgatggaacctggcagacagagcatggacctggtggagctggtgagcctcttccccactctggccggcctggctggcctgcaggtgccaccaagatgcccagtgccttctttccacgtggagctgtgtagagagggaaagaacctgctgaagcacttcagatttagagatctggaggaggatccctacctgccaggcaacccaagggagctgatcgcctacagccagtatcccagaccctctgatatcccccagtggaacagcgataagccctccctgaaagacatcaagattatgggctactccatcaggaccattgactaccggtacacagtgtgggtgggcttcaaccccgatgagtttctggccaacttcagcgacatccacgccggcgagctgtattttgtggactccgaccccctgcaggaccacaacatgtacaacgactcccagggcggcgacctgttccagctgctgatgccctgagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaacctgcaggtctagatacgtagataagtagcatggcgggttaatcattaactacaaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 59 - DNA, 4483 bp, synthetic construct
  misc_feature 1..4483: note = Synthetic polynucleotide.
  source 1..4483: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgcacacgtgacgcgtccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagcctcgaggaacgttttattttcccagagacaggctcagggacttctgggtgtagtggctgtgcagggcttcgtggagcacgctgcagctaaacacgttgccctcttgccatcggctcttgtccacggtcagccgtgaatacaggaagaagctgccatcgctgtccagcactggaggggtcgtcttgtagttgttttcaggctgtccgttgctctcccattcgacggcgatgtcgctggggtagaagcccttcaccaggcaggtcagagacacttggttctttgtcatttcttcttgagaaggaggcagtgtatagacttggggctctctgggctggcccttggcctttgagatggtcttctcgatgctgctaggcaggcccttgttagacaccttgcacttgtactctttgccgttcagccagtcctggtgcagcactgtcagcacggacaccacccggtaggtgctgttaaactgttcctcgcggggtttggtcttggcattatgaacttccactccgtcaacgtaccaattaaactgcacctcaggatcctcctgggacacatccaccaccacacatgtcacttcaggggtccggctgatcatcagggtgtctttgggctttggtgggaacaggaaaacagaagggccggcgactggtggagctggacatggtgggcattccacgcagcactttctctcaacggtcttatccactttggtgttgctaggcttgtggtccacattacaggtgtaggtctgtgtgccgaagttggagctaggaacggtgaccacgctactcagagaatacaggccagagctctgcagcacggctgggaaggtgtgcacgccgcttgtcagggcgccgctgttccagctcactgtcacgggttcggggaagtagtctttgaccagacaacccagggcggctgtgctttcagatgtgcttctgctgcatggggccagtgggaacacgctgggtcccttagtactggcgctgctgacggtgacaagggtgccttggccccacacgtcgaagtaccagttaggggatgagccaaaaaagtatctggcgcagtagtacacagctgtgtcctcgcttctcaggctgctcagttccatgtacacggtgcttgtggaggtatctctggtcatggtcactctatccttgaagttctctgtgtactcggtgtggccagatcctggcaggatctcgcccatccattccaggccttggccaggggcctgccgcacccactggatccagtagttgctgaagatgtggccgctggccttgcaggacaccttcacgctagctccgggctttttcacttcggctccgctttgaaccagctgcacttggctgtgcacgcctgtggcggtggccaccaggaacaggatgatgcaggaccagcccatggtggcactagttcagttccaaaggttggaatctaaaagagagaaacaattagaatcagtagtttaacacattatacacttaaaaattttatatttaccttagaggattcactgtcccaggtcagtggtggtgcctgaagctgaggagacagggccctgtcctcgtccgtatttaagcagtggatccagaggggcaacgggggaggctgctggtgaatattaaccaaggtcaccccagttatcggaggagcaaacaggggctaagtccactggctgggatctgagtcgcccgcctacgctgcccggacgctttgcctgggcagtgtacagcttccactgcacttaccgaaaggagtcattcccctctcacactacctaaaccacgccaggacaacctctgctcctctccaccgaaattccaaggggtcgagtggatgttggaggtggcatgggcccagagaggtctctgacctctgccccagctccaaggtcagcaggcagggagggctgtgtgtttgctgtttgctgcttgcaatgtttgcccattttagggccgcggcacgtgcttaaggcccccttttgcatccagtttattcctacatttgtcacactgttaacagcccaccccttccaatgagaccagtggtatcagtgagttgtggagatcaggaaaagggctcaagagaaaggcagtcaaagccctttttctgtccctgtcccagctgctttaataagatctccataagagaagagggacagctatgactgggagtagtcaggagaggaggaaaaatctggctagtaaaacatgtaaggaaaattttagggatgttaaagaaaaaaataacacaaaacaaaatataaaaaaaatctaacctcaagtcaaggcttttctatggaataaggaatggacagcagggggctgtttcatatactgatgacctctttatagccaacctttgttcatggcagccagcatatgggcatatgttgccaaactctaaaccaaatactcattctgatgttttaaatgatttgccctcccatatgtccttccgagtgagagacacaaaaaattccaacacactattgcaatgaaaataaatttcctttattagccagaagtcagatgctcaaggggcttcatgatgtccccataatttttggcagagggaaaaagatctccggagggggaggctgctggtgaatattaaccaaggtcaccccagttatcggaggagcaaacaggggctaagtccacctcgagccatggcgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatcagctagcgccggcaagaggtaagggtttaagggatggttggttggtggggtattaatgtttaattacctggagcacctgcctgaaatcactttttttcaggttggaccggtgccaccatggacatgagggtccctgctcagctgctggggctcctgctgctctggctcagcggtgccagatgtgatatccagatgacccagtctccatctagcctgtccgccagcgtgggcgacagagtgaccatcacctgcggcgccagcgagaacatctatggcgctctgaactggtaccagcagaaacctggcaaggcccctaagctgctgatctacggcgccaccaacctggccgatggcgtgcctagtagattcagcggatctggcagcggcacagacttcaccctgaccatcagcagcctgcaacctgaggactttgccacatactactgccagaacgtgctgaatacacctctgacattcggccaaggaaccaaagtggaaatcaagcggaccgtggccgctcctagcgtgttcatcttccctccttccgatgaacaactgaagagcggaaccgcctctgtggtgtgcctgctgaacaacttctaccctagagaggccaaggtgcagtggaaggtcgacaacgccctgcagagcggcaacagccaggagagcgtgacggaacaggacagcaaggacagcacctacagcctgagctccacccttacactgtctaaagccgactacgagaagcacaaggtgtacgcctgtgaagtgacacaccagggcctgagcagccctgtgaccaagtcttttaaccggggcgagtgctgaattcgaatcgtacctagggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaagcttgtttaaacgtacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

(19) United States
(12) Patent Application Publication
(10) Pub. No.: US 2023/0076072 A1
(43) Pub. Date: Mar. 9, 2023

(54) ADENO-ASSOCIATED VIRUS FORMULATIONS

(71) Applicant: Oxford Biomedica Solutions LLC, Bedford, MA (US)

(72) Inventors: Lori KARPES, Bedford, MA (US); Michael Mercaldi, Bedford, MA (US); Eric Faulkner, Bedford, MA (US)

(21) Appl. No.: 17/821,884

(22) Filed: Aug. 24, 2022

Related U.S. Application Data
(60) Provisional application No. 63/260,521, filed on Aug. 24, 2021.

(51) Int. Cl. A61K 48/00; A61K 47/22; A61K 47/26; A61K 47/02; A61K 47/10

(52) U.S. Cl. CPC A61K 48/0008; A61K 47/22; A61K 47/26; A61K 47/02; A61K 47/10

(57) ABSTRACT

Provided herein are pharmaceutical compositions (e.g., formulations) that can provide for the long-term stability of AAV vectors. Also provided herein are methods of making and using the pharmaceutical compositions. The pharmaceutical compositions provided by the present disclosure generally comprise an AAV, histidine, a stabilizing agent, a salt, and a surfactant.

### RELATED APPLICATIONS

This application claims priority to U.S. Provisional Patent Application Ser. No. 63/260,521, filed Aug. 24, 2021, the entire disclosure of which is hereby incorporated herein by reference.

### SEQUENCE LISTING

This application contains a sequence listing which has been submitted electronically in ASCII format and is hereby incorporated by reference in its entirety (said ASCII copy, created on Aug. 16, 2022, is named “HMW-047WO (192920) Sequence Listing ST26” and is 94,647 bytes in size).

### BACKGROUND

Gene therapy using adeno-associated virus (AAV) vectors has the potential to treat a wide variety of human disorders. One challenge with using AAV gene therapy vectors is maintaining the stability of the vector during long-term storage of the final drug formulation.

Accordingly, there is a need in the art for novel AAV formulations that can provide for the long-term stability of AAV vectors.

### SUMMARY

The present disclosure provides pharmaceutical compositions (e.g., formulations) that can provide for the long-term stability of AAV vectors. Also provided herein are methods of making and using the pharmaceutical compositions. The pharmaceutical compositions provided by the present disclosure generally comprise an AAV, histidine, a stabilizing agent (e.g., trehalose), a salt, and a surfactant. The pharmaceutical compositions described herein are particularly advantageous in that they enhance the stability of AAV vectors when challenged by thermal stress, including, for example, reducing the extent of vector aggregation, maintaining high vector genome titers, and maintaining AAV vector purity over time. Consequently, the pharmaceutical compositions provided herein are particularly suitable for the long-term storage of AAV vectors (e.g., AAV gene therapy vectors).

Accordingly, in one aspect, the present disclosure provides a pharmaceutical composition comprising: (a) an adeno-associated virus (AAV); (b) histidine; (c) trehalose; and (d) greater than about 150 mM sodium chloride.

In certain embodiments, the pharmaceutical composition comprises about 5 mM to about 50 mM histidine. In certain embodiments, the pharmaceutical composition comprises about 20 mM histidine.

In certain embodiments, the pharmaceutical composition comprises about 1% (w/v) to about 10% (w/v) trehalose. In certain embodiments, the pharmaceutical composition comprises about 1% (w/v) trehalose. In certain embodiments, the pharmaceutical composition comprises about 3% (w/v) trehalose. In certain embodiments, the pharmaceutical composition comprises about 5% (w/v) trehalose.

In certain embodiments, the pharmaceutical composition comprises no more than about 200 mM sodium chloride. In certain embodiments, the pharmaceutical composition comprises about 175 mM sodium chloride. In certain embodiments, the pharmaceutical composition comprises about 200 mM sodium chloride.

In certain embodiments, the pharmaceutical composition comprises about 0.01% (w/v) to about 0.05% (w/v) Poloxamer 188. In certain embodiments, the pharmaceutical composition comprises about 0.03% (w/v) Poloxamer 188.

In certain embodiments, the pharmaceutical composition comprises: (a) an adeno-associated virus (AAV); (b) about 20 mM histidine; (c) about 3% (w/v) trehalose; (d) about 0.03% (w/v) Poloxamer 188; and (e) about 175 mM sodium chloride.

In certain embodiments, the pH of the pharmaceutical composition is from about 6 to about 8. In certain embodiments, the pH of the pharmaceutical composition is from about 6.3 to about 8.3. In certain embodiments, the pH of the pharmaceutical composition is about 7.3.

In certain embodiments, the pharmaceutical composition comprises at least about 1e13 vg/mL of the AAV. In certain embodiments, the pharmaceutical composition comprises about 1e13 vg/mL to about 6e15 vg/mL of the AAV. In certain embodiments, the pharmaceutical composition comprises about 1e13 vg/mL to about 1e15 vg/mL of the AAV. In certain embodiments, the pharmaceutical composition comprises about 2e13 vg/mL of the AAV. In certain embodiments, the pharmaceutical composition comprises about 6e13 vg/mL of the AAV. In certain embodiments, the pharmaceutical composition comprises about 8e13 vg/mL of the AAV. In certain embodiments, the pharmaceutical composition comprises about 1e14 vg/mL of the AAV. In certain embodiments, the pharmaceutical composition comprises about 2e14 vg/mL of the AAV. In certain embodiments, the pharmaceutical composition comprises at least about 1e15 vg/mL of the AAV.

In certain embodiments, the AAV is a recombinant AAV (rAAV) comprising an rAAV genome comprising a transgene. In certain embodiments, the transgene encodes a polypeptide. In certain embodiments, the transgene encodes an miRNA, shRNA, siRNA, antisense RNA, gRNA, antagomir, miRNA sponge, RNA aptazyme, RNA aptamer, lncRNA, ribozyme, or mRNA. In certain embodiments, the transgene encodes a protein selected from the group consisting of phenylalanine hydroxylase (PAH), glucose-6-phosphatase (G6Pase), iduronate-2-sulfatase (I2S), arylsulfatase A (ARSA), and frataxin (FXN). In certain embodiments, the transgene encodes a protein selected from the group consisting of glucose-6-phosphatase (G6Pase) and frataxin (FXN). In certain embodiments, the transgene encodes a protein which is not selected from the group consisting of phenylalanine hydroxylase (PAH), iduronate-2-sulfatase (I2S), arylsulfatase A (ARSA), and an anti-complement component 5 antibody.

In certain embodiments, the rAAV genome further comprises a transcriptional regulatory element operably linked to the transgene. In certain embodiments, the transcriptional regulatory element comprises a promoter element and/or an intron element.

In certain embodiments, the rAAV genome further comprises a polyadenylation sequence. In certain embodiments, the polyadenylation sequence is 3′ to the transgene.

In certain embodiments, the rAAV genome comprises a nucleotide sequence that is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 50, 51, 52, 53, or 54.

In certain embodiments, the rAAV genome further comprises a 5′ inverted terminal repeat (5′ ITR) nucleotide sequence 5′ of the transgene, and a 3′ inverted terminal repeat (3′ ITR) nucleotide sequence 3′ of the transgene. In certain embodiments, the 5′ ITR nucleotide sequence is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 39, 41, or 42, and/or the 3′ ITR nucleotide sequence is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 40, 43, or 44.

In certain embodiments, the rAAV genome comprises a nucleotide sequence that is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 55, 56, 57, 58, or 59.

In certain embodiments, the rAAV comprises an AAV capsid comprising an AAV capsid protein. In certain embodiments, the AAV capsid protein is selected from the group consisting of AAV1, AAV2, AAV3, AAV4, AAV5, AAV6, AAV7, AAV8, AAV9, AAV-DJ, AAV-LK03, NP59, VOY101, VOY201, VOY701, VOY801, VOY1101, AAVPHP.N, AAVPHP.A, AAVPHP.B, PHP.B2, PHP.B3, G2A3, G2B4, G2B5, and PUP.S.

In certain embodiments, the AAV capsid protein does not comprise an amino acid sequence that is at least 85%, 86%, 87%, 88%, 89%, 90%, 91%, 92%, 93%, 94%, 95%, 96%, 97%, 98%, 99%, or 100% identical to the amino acid sequence of amino acids 203-736 of SEQ ID NO: 1, 2, 3, 4, 5, 6, 7, 8, 9, 10, 11, 12, 13, 15, 16, or 17, wherein: the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the AAV capsid protein does not comprise an amino acid sequence that is at least 85%, 86%, 87%, 88%, 89%, 90%, 91%, 92%, 93%, 94%, 95%, 96%, 97%, 98%, 99%, or 100% identical to the amino acid sequence of amino acids 203-736 of SEQ ID NO: 1, 2, 3, 4, 5, 6, 7, 8, 9, 10, 11, 12, 13, 15, 16, or 17, wherein: (a) the amino acid in the capsid protein corresponding to amino acid 626 of SEQ ID NO: 16 is G, and the amino acid in the capsid protein corresponding to amino acid 718 of SEQ ID NO: 16 is G; (b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (c) the amino acid in the capsid protein corresponding to amino acid 505 of SEQ ID NO: 16 is R, and the amino acid in the capsid protein corresponding to amino acid 687 of SEQ ID NO: 16 is R; (d) the amino acid in the capsid protein corresponding to amino acid 346 of SEQ ID NO: 16 is A, and the amino acid in the capsid protein corresponding to amino acid 505 of SEQ ID NO: 16 is R; or (e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the AAV capsid protein does not comprise the amino acid sequence of amino acids 203-736 of SEQ ID NO: 1, 2, 3, 4, 5, 6, 7, 8, 9, 10, 11, 12, 13, 15, 16, or 17.

In certain embodiments, the AAV capsid protein does not comprise an amino acid sequence that is at least 85%, 86%, 87%, 88%, 89%, 90%, 91%, 92%, 93%, 94%, 95%, 96%, 97%, 98%, 99%, or 100% identical to the amino acid sequence of amino acids 138-736 of SEQ ID NO: 1, 2, 3, 4, 5, 6, 7, 8, 9, 10, 11, 12, 13, 15, 16, or 17, wherein: the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the AAV capsid protein does not comprise an amino acid sequence that is at least 85%, 86%, 87%, 88%, 89%, 90%, 91%, 92%, 93%, 94%, 95%, 96%, 97%, 98%, 99%, or 100% identical to the amino acid sequence of amino acids 138-736 of SEQ ID NO: 1, 2, 3, 4, 5, 6, 7, 8, 9, 10, 11, 12, 13, 15, 16, or 17, wherein: (a) the amino acid in the capsid protein corresponding to amino acid 626 of SEQ ID NO: 16 is G, and the amino acid in the capsid protein corresponding to amino acid 718 of SEQ ID NO: 16 is G; (b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (c) the amino acid in the capsid protein corresponding to amino acid 505 of SEQ ID NO: 16 is R, and the amino acid in the capsid protein corresponding to amino acid 687 of SEQ ID NO: 16 is R; (d) the amino acid in the capsid protein corresponding to amino acid 346 of SEQ ID NO: 16 is A, and the amino acid in the capsid protein corresponding to amino acid 505 of SEQ ID NO: 16 is R; or (e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the AAV capsid protein does not comprise the amino acid sequence of amino acids 138-736 of SEQ ID NO: 1, 2, 3, 4, 5, 6, 7, 9, 10, 11, 12, 13, 15, 16, or 17.

In certain embodiments, the AAV capsid protein does not comprise an amino acid sequence that is at least 85%, 86%, 87%, 88%, 89%, 90%, 91%, 92%, 93%, 94%, 95%, 96%, 97%, 98%, 99%, or 100% identical to the amino acid sequence of amino acids 1-736 of SEQ ID NO: 1, 2, 3, 4, 5, 6, 7, 8, 9, 10, 11, 12, 13, 15, 16, or 17, wherein: the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the AAV capsid protein does not comprise an amino acid sequence that is at least 85%, 86%, 87%, 88%, 89%, 90%, 91%, 92%, 93%, 94%, 95%, 96%, 97%, 98%, 99%, or 100% identical to the amino acid sequence of amino acids 1-736 of SEQ ID NO: 1, 2, 3, 4, 5, 6, 7, 8, 9, 10, 11, 12, 13, 15, 16, or 17, wherein: (a) the amino acid in the capsid protein corresponding to amino acid 2 of SEQ ID NO: 16 is T, and the amino acid in the capsid protein corresponding to amino acid 312 of SEQ ID NO: 16 is Q; (b) the amino acid in the capsid protein corresponding to amino acid 65 of SEQ ID NO: 16 is I, and the amino acid in the capsid protein corresponding to amino acid 626 of SEQ ID NO: 16 is Y; (c) the amino acid in the capsid protein corresponding to amino acid 77 of SEQ ID NO: 16 is R, and the amino acid in the capsid protein corresponding to amino acid 690 of SEQ ID NO: 16 is K; (d) the amino acid in the capsid protein corresponding to amino acid 119 of SEQ ID NO: 16 is L, and the amino acid in the capsid protein corresponding to amino acid 468 of SEQ ID NO: 16 is S; (e) the amino acid in the capsid protein corresponding to amino acid 626 of SEQ ID NO: 16 is G, and the amino acid in the capsid protein corresponding to amino acid 718 of SEQ ID NO: 16 is G; (f) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (g) the amino acid in the capsid protein corresponding to amino acid 505 of SEQ ID NO: 16 is R, and the amino acid in the capsid protein corresponding to amino acid 687 of SEQ ID NO: 16 is R; (h) the amino acid in the capsid protein corresponding to amino acid 346 of SEQ ID NO: 16 is A, and the amino acid in the capsid protein corresponding to amino acid 505 of SEQ ID NO: 16 is R; or (i) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the AAV capsid protein does not comprise the amino acid sequence of amino acids 1-736 of SEQ ID NO: 1, 2, 3, 4, 5, 6, 7, 8, 9, 10, 11, 12, 13, 15, 16, or 17.

In certain embodiments, the AAV capsid protein comprises an amino acid sequence that is at least 85%, 86%, 87%, 88%, 89%, 90%, 91%, 92%, 93%, 94%, 95%, 96%, 97%, 98%, 99%, or 100% identical to the amino acid sequence of amino acids 203-736 of SEQ ID NO: 1, 2, 3, 4, 5, 6, 7, 8, 9, 10, 11, 12, 13, 15, 16, or 17. In certain embodiments, the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, (a) the amino acid in the capsid protein corresponding to amino acid 626 of SEQ ID NO: 16 is G, and the amino acid in the capsid protein corresponding to amino acid 718 of SEQ ID NO: 16 is G; (b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (c) the amino acid in the capsid protein corresponding to amino acid 505 of SEQ ID NO: 16 is R, and the amino acid in the capsid protein corresponding to amino acid 687 of SEQ ID NO: 16 is R; (d) the amino acid in the capsid protein corresponding to amino acid 346 of SEQ ID NO: 16 is A, and the amino acid in the capsid protein corresponding to amino acid 505 of SEQ ID NO: 16 is R; or (e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the AAV capsid protein comprises the amino acid sequence of amino acids 203-736 of SEQ ID NO: 1, 2, 3, 4, 5, 6, 7, 8, 9, 10, 11, 12, 13, 15, 16, or 17.

In certain embodiments, the AAV capsid protein comprises an amino acid sequence that is at least 85%, 86%, 87%, 88%, 89%, 90%, 91%, 92%, 93%, 94%, 95%, 96%, 97%, 98%, 99%, or 100% identical to the amino acid sequence of amino acids 138-736 of SEQ ID NO: 1, 2, 3, 4, 5, 6, 7, 8, 9, 10, 11, 12, 13, 15, 16, or 17. In certain embodiments, the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, (a) the amino acid in the capsid protein corresponding to amino acid 626 of SEQ ID NO: 16 is G, and the amino acid in the capsid protein corresponding to amino acid 718 of SEQ ID NO: 16 is G; (b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (c) the amino acid in the capsid protein corresponding to amino acid 505 of SEQ ID NO: 16 is R, and the amino acid in the capsid protein corresponding to amino acid 687 of SEQ ID NO: 16 is R; (d) the amino acid in the capsid protein corresponding to amino acid 346 of SEQ ID NO: 16 is A, and the amino acid in the capsid protein corresponding to amino acid 505 of SEQ ID NO: 16 is R; or (e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the AAV capsid protein comprises the amino acid sequence of amino acids 138-736 of SEQ ID NO: 1, 2, 3, 4, 5, 6, 7, 9, 10, 11, 12, 13, 15, 16, or 17.

In certain embodiments, the AAV capsid protein comprises an amino acid sequence that is at least 85%, 86%, 87%, 88%, 89%, 90%, 91%, 92%, 93%, 94%, 95%, 96%, 97%, 98%, 99%, or 100% identical to the amino acid sequence of amino acids 1-736 of SEQ ID NO: 1, 2, 3, 4, 5, 6, 7, 8, 9, 10, 11, 12, 13, 15, 16, or 17. In certain embodiments, the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, (a) the amino acid in the capsid protein corresponding to amino acid 2 of SEQ ID NO: 16 is T, and the amino acid in the capsid protein corresponding to amino acid 312 of SEQ ID NO: 16 is Q; (b) the amino acid in the capsid protein corresponding to amino acid 65 of SEQ ID NO: 16 is I, and the amino acid in the capsid protein corresponding to amino acid 626 of SEQ ID NO: 16 is Y; (c) the amino acid in the capsid protein corresponding to amino acid 77 of SEQ ID NO: 16 is R, and the amino acid in the capsid protein corresponding to amino acid 690 of SEQ ID NO: 16 is K; (d) the amino acid in the capsid protein corresponding to amino acid 119 of SEQ ID NO: 16 is L, and the amino acid in the capsid protein corresponding to amino acid 468 of SEQ ID NO: 16 is S; (e) the amino acid in the capsid protein corresponding to amino acid 626 of SEQ ID NO: 16 is G, and the amino acid in the capsid protein corresponding to amino acid 718 of SEQ ID NO: 16 is G; (f) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M; (g) the amino acid in the capsid protein corresponding to amino acid 505 of SEQ ID NO: 16 is R, and the amino acid in the capsid protein corresponding to amino acid 687 of SEQ ID NO: 16 is R; (h) the amino acid in the capsid protein corresponding to amino acid 346 of SEQ ID NO: 16 is A, and the amino acid in the capsid protein corresponding to amino acid 505 of SEQ ID NO: 16 is R; or (i) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the AAV capsid protein comprises the amino acid sequence of amino acids 1-736 of SEQ ID NO: 1, 2, 3, 4, 5, 6, 7, 8, 9, 10, 11, 12, 13, 15, 16, or 17.

In another aspect, the present disclosure provides a method of transducing a target cell in a subject, comprising administering to the subject a therapeutically effective amount of a pharmaceutical composition described herein under conditions whereby the target cell is transduced.

In another aspect, the present disclosure provides a method of expressing a transgene in a target cell in a subject, comprising administering to the subject a therapeutically effective amount of a pharmaceutical composition described herein under conditions whereby the target cell is transduced and the transgene is expressed.

In certain embodiments, the target cell is a cell of the blood, liver, heart, joint tissue, muscle, brain, kidney, or lung. In certain embodiments, the target cell is a cell of the central nervous system, or the peripheral nervous system.

In another aspect, the present disclosure provides a method of treating or preventing a disease in a subject in need thereof, comprising administering to the subject a therapeutically effective amount of a pharmaceutical composition described herein.

In certain embodiments, the formulation is administered to the subject intravenously, intraperitoneally, subcutaneously, intramuscularly, intrathecally, intracerebroventricularly, intradermally, or directly into the central nervous system of the subject.

In certain embodiments, the subject is a human subject.

In another aspect, the present disclosure provides a method for the preparation of a pharmaceutical composition described herein.

### BRIEF DESCRIPTION OF DRAWINGS

FIG. 1 is a graph showing the increase in percentage aggregation of AAV vectors maintained in Formulations 1-3 for two weeks at 40° C. The components of Formulations 1-3 are set forth in Table 1.

FIG. 2 is a graph showing the increase in percentage aggregation of AAV vectors maintained in Formulations 4-6 for two weeks at 40° C. The components of Formulations 4-6 are set forth in Table 1.

FIGS. 3A-3D are graphs showing the level of vector genome titers in Formulations 1, 7, 8, 9, and 10 at 25° C. (FIGS. 3A and 3B) and 40° C. (FIGS. 3C and 3D) over time. The components of Formulations 1, 7, 8, 9, and 10 are set forth in Tables 1 and 2.

FIGS. 4A-4D are graphs showing the level of AAV vector purity (% Purity) in Formulations 1, 7, 8, 9, and 10 at 25° C. (FIGS. 4A and 4B) and 40° C. (FIGS. 4C and 4D) over time. The components of Formulations 1, 7, 8, 9, and 10 are set forth in Table 2.

FIGS. 5A-5D are graphs showing the level of VP1 capsid protein integrity measured by CE-SDS as a function of corrected area under the curve for VP1 (% Total Area) in Formulations 1, 7, 8, 9, and 10 at 25° C. (FIGS. 5A and 5B) and 40° C. (FIGS. 5C and 5D) over time. The components of Formulations 1, 7, 8, 9, and 10 are set forth in Tables 1 and 2.

FIGS. 6A-6D are graphs showing the level of AAV vector aggregates as a function of high molecular weight species (% HMW) in Formulations 1, 7, 8, 9, and 10 at 25° C. (FIGS. 6A and 6B) and 40° C. (FIGS. 6C and 6D) over time. The components of Formulations 1, 7, 8, 9, and 10 are set forth in Tables 1 and 2.

FIGS. 7A-7D are graphs showing the level of AAV vector aggregates as a function of high molecular weight species (% HMW) in Formulations 1, 8, 9, and 10 to reduce the increase in vector aggregates when incubated at 25° C. (FIGS. 7A and 7B) and 40° C. (FIGS. 7C and 7D) over time. The components of Formulations 1, 8, 9, and 10 are set forth in Tables 1 and 2.

FIGS. 8A-8D are graphs showing the level of vector genome titers in Formulations 1 and 9 at −80° C. (FIG. 8A), 5° C. (FIG. 8B), 25° C. (FIG. 8C), and 40° C. (FIG. 8D). The components of Formulations 1 and 9 are set forth in Tables 1 and 2.

FIGS. 9A-9D are graphs showing the level of AAV vector aggregates as a function of high molecular weight species (% HMW) in Formulations 1 and 9 at −80° C. (FIG. 9A), 5° C. (FIG. 9B), 25° C. (FIG. 9C), and 40° C. (FIG. 9D). The components of Formulations 1 and 9 are set forth in Tables 1 and 2.

FIGS. 10A-10D are graphs showing the level of AAV vector purity (% Purity) in Formulations 1 and 9 at −80° C. (FIG. 10A), 5° C. (FIG. 10B), 25° C. (FIG. 10C), and 40° C. (FIG. 10D). The components of Formulations 1 and 9 are set forth in Tables 1 and 2.

FIGS. 11A-11D are graphs showing the level of VP1 capsid protein integrity measured by CE-SDS as a function of corrected area under the curve for VP1 (% Total Area) in Formulations 1 and 9 at −80° C. (FIG. 11A), 5° C. (FIG. 11B), 25° C. (FIG. 11C), and 40° C. (FIG. 11D). The components of Formulations 1 and 9 are set forth in Tables 1 and 2.

FIGS. 12A-12F are graphs showing the level of vector genome titers (FIG. 12A), capsid titers (FIG. 12B), AAV vector aggregates as a function of high molecular weight species (% HMW) (FIG. 12C), AAV vector purity (% Purity) (FIG. 12D), VP1 capsid protein integrity measured by CE-SDS as a function of corrected area under the curve for VP1 (% Total Area) (FIG. 12E), and AAV vector potency measured by relative gene expression (% RGE) (FIG. 12F) in Formulation 9 at −80° C., 5° C., and 25° C. The components of Formulation 9 are set forth in Table 2.

FIGS. 13A-13C are graphs showing the level of subvisible particulate counts over time for formulations tested at −80° C., 5° C., and 25° C.

FIGS. 14A-14F are graphs showing the level of vector genome titers (FIG. 14A), capsid titers (FIG. 14B), AAV vector aggregates as a function of high molecular weight species (% HMW) (FIG. 14C), AAV potency measured by relative gene expression (% RGE) (FIG. 14D), AAV vector purity (% Purity) (FIG. 14E), VP1 capsid protein integrity measured by CE-SDS as a function of corrected area under the curve for VP1 (% Total Area) (FIG. 14F) in Formulation 9 across multiple freeze-thaw cycles and at pH values from 6.3 to 8.3. The components of Formulation 9 are set forth in Table 2.

FIGS. 15A-15K are graphs showing the level of vector genome titers (FIG. 15A), capsid titers (FIG. 15C), AAV vector aggregates as a function of high molecular weight species (% HMW) (FIG. 15. E), AAV vector purity (% Purity) (FIG. 15G), VP1 capsid protein integrity measured by CE-SDS as a function of corrected area under the curve for VP1 (% Total Area) (FIG. 15I), and AAV potency measured by relative gene expression (% RGE) (FIG. 15K) in Formulation 9 at 5° C. at various pH values; and the level of vector genome titers (FIG. 15B), capsid titers (FIG. 15D), AAV vector aggregates as a function of high molecular weight species (% HMW) (FIG. 15F), AAV vector purity (% Purity) (FIG. 15H), and VP1 capsid protein integrity measured by CE-SDS as a function of corrected area under the curve for VP1 (% Total Area) (FIG. 15J) in Formulation 9 at 25° C. at various pH values. The components of Formulation 9 are set forth in Table 2.

FIGS. 16A-16C are a table (FIG. 16A) and graphs (FIG. 16B-C) showing the level of AAV vector aggregates as a function of high molecular weight species (% HMW) (FIG. 16A), diameter on Dynamic Light Scattering (DLS) (FIG. 16B), and diameter on Polydispersity Index (PDI) (FIG. 16C) in various concentrations (vg/mL, capsids/mL) of Formulation 9 at 2-8° C. over 90 days. The components of Formulation 9 are set forth in Table 2.

FIG. 17 is a table showing the melting temperature and the level of AAV vector aggregates as a function of particle diameter, on Dynamic Light Scattering (DLS) and Polydispersity Index (PDI), of various capsid serotypes in Formulation 9. The components of Formulation 9 are set forth in Table 2.

### DETAILED DESCRIPTION

The present disclosure provides pharmaceutical compositions (e.g., formulations) that can provide for the long-term stability of AAV vectors. Also provided herein are methods of making and using the pharmaceutical compositions. The pharmaceutical compositions provided by the present disclosure generally comprise an AAV, a buffering agent (e.g., histidine), a stabilizing agent (e.g., trehalose), a salt, and a surfactant. The pharmaceutical compositions described herein are particularly advantageous in that they enhance the stability of AAV vectors when challenged by thermal stress, including, for example, reducing the extent of vector aggregation, maintaining high vector genome titers, and maintaining AAV vector purity over time.

### I. Definitions

As used herein, the term “recombinant adeno-associated virus” or “rAAV” refers to an adeno-associated virus (AAV) comprising a genome lacking functional rep and cap genes.

As used herein, the term “cap gene” refers to a nucleic acid sequence that encodes an AAV capsid protein.

As used herein, the term “rAAV genome” refers to a nucleic acid molecule comprising the genome sequence of an rAAV. The skilled artisan will appreciate that where an rAAV genome comprises a transgene, the rAAV genome can be in the sense or antisense orientation relative to the direction of transcription of the transgene.

As used herein, the term “editing genome” refers to a recombinant AAV genome that is capable of integrating an editing element (e.g., one or more nucleotides or an internucleotide bond) via homologous recombination into a target locus to correct a genetic defect in a target gene. The skilled artisan will appreciate that the portion of an editing genome comprising the 5′ homology arm, editing element, and 3′ homology arm can be in the sense or antisense orientation relative to the target locus.

As used herein, the term “editing element” refers to the portion of an editing genome that when integrated at a target locus modifies the target locus. An editing element can mediate insertion, deletion, or substitution of one or more nucleotides at the target locus. As used herein, the term “target locus” refers to a region of a chromosome or an internucleotide bond (e.g., a region or an internucleotide bond of a target gene) that is modified by an editing element.

As used herein, the term “homology arm” refers to a portion of an editing genome positioned 5′ or 3′ of an editing element that is substantially identical to the genome flanking a target locus.

As used herein, the “percentage identity” between two nucleotide sequences or between two amino acid sequences is calculated by multiplying the number of matches between the pair of aligned sequences by 100, and dividing by the length of the aligned region, including internal gaps. Identity scoring only counts perfect matches and does not consider the degree of similarity of amino acids to one another. Note that only internal gaps are included in the length, not gaps at the sequence ends.

As used herein, the term “coding sequence” refers to the portion of a complementary DNA (cDNA) that encodes a polypeptide, starting at the start codon and ending at the stop codon. A gene may have one or more coding sequences due to alternative splicing, alternative translation initiation, and variation within the population. A coding sequence may be wild-type or a non-naturally occurring variant (e.g., a codon optimized variant).

As used herein, the term “transcriptional regulatory element” or “TRE” refers to a cis-acting nucleotide sequence, for example, a DNA sequence, that regulates (e.g., controls, increases, or reduces) transcription of an operably linked nucleotide sequence by an RNA polymerase to form an RNA molecule. A TRE relies on one or more trans-acting molecules, such as transcription factors, to regulate transcription. Thus, one TRE may regulate transcription in different ways when it is in contact with different trans-acting molecules, for example, when it is in different types of cells. A TRE may comprise one or more promoter elements and/or enhancer elements. A skilled artisan would appreciate that the promoter and enhancer elements in a gene may be close in location, and the term “promoter” may refer to a sequence comprising a promoter element and an enhancer element. Thus, the term “promoter” does not exclude an enhancer element in the sequence. The promoter and enhancer elements do not need to be derived from the same gene or species, and the sequence of each promoter or enhancer element may be either identical or substantially identical to the corresponding endogenous sequence in the genome.

As used herein, the term “operably linked” is used to describe the connection between a TRE and a coding sequence to be transcribed. Typically, gene expression is placed under the control of a TRE comprising one or more promoter and/or enhancer elements. The coding sequence is “operably linked” to the TRE if the transcription of the coding sequence is controlled or influenced by the TRE. The promoter and enhancer elements of the TRE may be in any orientation and/or distance from the coding sequence, as long as the desired transcriptional activity is obtained. In certain embodiments, the TRE is upstream from the coding sequence.

As used herein, the term “polyadenylation sequence” refers to a DNA sequence that, when transcribed into RNA, constitutes a polyadenylation signal sequence. The polyadenylation sequence can be native or exogenous. The exogenous polyadenylation sequence can be a mammalian or a viral polyadenylation sequence (e.g., an SV40 polyadenylation sequence).

As used herein, “exogenous polyadenylation sequence” refers to a polyadenylation sequence not identical or substantially identical to the endogenous polyadenylation sequence of a transgene. In certain embodiments, an exogenous polyadenylation sequence is a polyadenylation sequence of a gene different from the transgene, but within the same species (e.g., human). In certain embodiments, an exogenous polyadenylation sequence is a polyadenylation sequence of a different organism (e.g., a virus).

### II. Recombinant Adeno-Associated Virus

Pharmaceutical compositions (e.g., formulations) disclosed herein comprise an adeno-associated virus (AAV). In certain embodiments, the pharmaceutical compositions comprise a recombinant adeno-associated virus (rAAV). As exemplified in the experimental Examples herein, pharmaceutical compositions of the present disclosure provide long-term stability and allow for high titer AAV formulations. The pharmaceutical compositions described herein enhance the stability of AAV vectors when challenged by thermal stress, including, for example, reducing the extent of vector aggregation, maintaining high vector genome titers, and maintaining AAV vector purity over time.

rAAV Genome

In certain embodiments, the pharmaceutical compositions of the present disclosure comprise a recombinant adeno-associated virus (rAAV) comprising an rAAV genome. In certain embodiments, the rAAV genome comprises a transgene.

In certain embodiments, the transgene comprises one or more sequences encoding an RNA molecule. Suitable RNA molecules include, without limitation, miRNA, shRNA, siRNA, antisense RNA, gRNA, antagomirs, miRNA sponges, RNA aptazymes, RNA aptamers, mRNA, lncRNAs, ribozymes, and synthetic RNAs known in the art.

certain embodiments, the transgene encodes one or more polypeptides, or a fragment thereof. Such transgenes can comprise the complete coding sequence of a polypeptide, or only a fragment of a coding sequence of a polypeptide. In certain embodiments, the transgene encodes a polypeptide that is useful to treat a disease or disorder in a subject. Suitable polypeptides include, without limitation, β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a), transforming growth factor beta (TGF-β), and the like; soluble receptors, such as soluble TNF-a receptors, soluble interleukin receptors (e.g., soluble IL-1 receptors and soluble type II IL-1 receptors), soluble γ/Δ T cell receptors, ligand-binding fragments of a soluble receptor, and the like; enzymes, such as a-glucosidase, imiglucerase, β-glucocerebrosidase, and alglucerase; enzyme activators, such as tissue plasminogen activator; chemokines, such as IP-10, monokine induced by interferon-gamma (Mig), Groα/IL-8, RANTES, MIP-1a, MIP-1β, MCP-1, PF-4, and the like; angiogenic agents, such as vascular endothelial growth factors (VEGFs, e.g., VEGF121, VEGF165, VEGF-C, VEGF-2), glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastrin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); tissue factors; macrophage activating factors; tumor necrosis factor (TNF); neutrophil chemotactic factor (NCF); tissue inhibitors of metalloproteinases; vasoactive intestinal peptide; angiogenin; angiotrofin; fibrin; hirudin; IL-1 receptor antagonists; ciliary neurotrophic factor (CNTF); brain-derived neurotrophic factor (BDNF); neurotrophins 3 and 4/5 (NT-3 and -4/5); glial cell derived neurotrophic factor (GDNF); aromatic amino acid decarboxylase (AADC); Factor VIII, Factor IX, Factor X; dystrophin or mini-dystrophin; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase, glucose transporter, aldolase A, β-enolase, glycogen synthase; lysosomal enzymes, such as iduronate-2-sulfatase (12S), and arylsulfatase A; and mitochondrial proteins, such as frataxin.

In certain embodiments, the transgene encodes one or more polypeptides, or a fragment thereof. Such transgenes can comprise the complete coding sequence of a polypeptide, or only a fragment of a coding sequence of a polypeptide. In certain embodiments, the transgene encodes a polypeptide that is useful to treat a disease or disorder in a subject. Suitable polypeptides include, without limitation, β-globin, hemoglobin, tissue plasminogen activator, and coagulation factors; colony stimulating factors (CSF); interleukins, such as IL-1, IL-2, IL-3, IL-4, IL-5, IL-6, IL-7, IL-8, IL-9, etc.; growth factors, such as keratinocyte growth factor (KGF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a), transforming growth factor beta (TGF-0), and the like; soluble receptors, such as soluble TNF-a receptors, soluble interleukin receptors (e.g., soluble IL-1 receptors and soluble type II IL-1 receptors), soluble γ/Δ T cell receptors, ligand-binding fragments of a soluble receptor, and the like; enzymes, such as a-glucosidase, imiglucerase, β-glucocerebrosidase, and alglucerase; enzyme activators, such as tissue plasminogen activator; chemokines, such as IP-10, monokine induced by interferon-gamma (Mig), Groα/IL-8, RANTES, MIP-1a, MIP-1β, MCP-1, PF-4, and the like; angiogenic agents, such as vascular endothelial growth factors (VEGFs, e.g., VEGF121, VEGF165, VEGF-C, VEGF-2), glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastrin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); tissue factors; macrophage activating factors; tumor necrosis factor (TNF); neutrophil chemotactic factor (NCF); tissue inhibitors of metalloproteinases; vasoactive intestinal peptide; angiogenin; angiotrofin; fibrin; hirudin; IL-1 receptor antagonists; ciliary neurotrophic factor (CNTF); brain-derived neurotrophic factor (BDNF); neurotrophins 3 and 4/5 (NT-3 and -4/5); glial cell derived neurotrophic factor (GDNF); aromatic amino acid decarboxylase (AADC); Factor VIII, Factor IX, Factor X; dystrophin or mini-dystrophin; lysosomal acid lipase; glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase, glucose transporter, aldolase A, β-enolase, glycogen synthase; lysosomal enzymes; and mitochondrial proteins, such as frataxin.

In certain embodiments, the transgene encodes a protein that may be defective in one or more lysosomal storage diseases. Suitable proteins include, without limitation, α-sialidase, cathepsin A, α-mannosidase, β-mannosidase, glycosylasparaginase, α-fucosidase, α-N-acetylglucosaminidase, β-galactosidase, β-hexosaminidase α-subunit, β-hexosaminidase β-subunit, GM2 activator protein, glucocerebrosidase, Saposin C, Arylsulfatase A, Saposin B, formyl-glycine generating enzyme, β-galactosylceramidase, α-galactosidase A, iduronate sulfatase, α-iduronidase, heparan N-sulfatase, acetyl-CoA transferase, N-acetyl glucosaminidase, β-glucuronidase, N-acetyl glucosamine 6-sulfatase, N-acetylgalactosamine 4-sulfatase, galactose 6-sulfatase, hyaluronidase, α-glucosidase, acid sphingomyelinase, acid ceramidase, acid lipase, cathepsin K, tripeptidyl peptidase, palmitoyl-protein thioesterase, cystinosin, sialin, UDP-N-acetylglucosamine, phosphotransferase γ-subunit, mucolipin-1, LAMP-2, NPC1, CLN 3, CLN 6, CLN 8, LYST, MYOV, RAB27A, melanophilin, and AP3 β-subunit. In certain embodiments, the transgene encodes a protein which is not selected from the group consisting of phenylalanine hydroxylase (PAH), iduronate-2-sulfatase (12S), arylsulfatase A (ARSA), and an anti-complement component 5 antibody.

In certain embodiments, the transgene encodes an antibody or a fragment thereof (e.g., a Fab, scFv, or full-length antibody). Suitable antibodies include, without limitation, muromonab-cd3, efalizumab, tositumomab, daclizumab, nebacumab, catumaxomab, edrecolomab, abciximab, rituximab, basiliximab, palivizumab, infliximab, trastuzumab, adalimumab, ibritumomab tiuxetan, omalizumab, cetuximab, bevacizumab, natalizumab, panitumumab, ranibizumab, eculizumab, certolizumab, ustekinumab, canakinumab, golimumab, ofatumumab, tocilizumab, denosumab, belimumab, ipilimumab, brentuximab vedotin, pertuzumab, raxibacumab, obinutuzumab, alemtuzumab, siltuximab, ramucirumab, vedolizumab, blinatumomab, nivolumab, pembrolizumab, idarucizumab, necitumumab, dinutuximab, secukinumab, mepolizumab, alirocumab, evolocumab, daratumumab, elotuzumab, ixekizumab, reslizumab, olaratumab, bezlotoxumab, atezolizumab, obiltoxaximab, inotuzumab ozogamicin, brodalumab, guselkumab, dupilumab, sarilumab, avelumab, ocrelizumab, emicizumab, benralizumab, gemtuzumab ozogamicin, durvalumab, burosumab, erenumab, galcanezumab, lanadelumab, mogamulizumab, tildrakizumab, cemiplimab, fremanezumab, ravulizumab, emapalumab, ibalizumab, moxetumomab, caplacizumab, romosozumab, risankizumab, polatuzumab, eptinezumab, leronlimab, sacituzumab, brolucizumab, isatuximab, and teprotumumab. In certain embodiments, the transgene encodes an antibody which is not an anti-complement component 5 antibody or a fragment thereof.

In certain embodiments, the transgene encodes a nuclease. Suitable nucleases include, without limitation, zinc fingers nucleases (ZFN) (see, e.g., Porteus and Baltimore (2003) Science 300: 763; Miller et al. (2007) Nat. Biotechnol. 25:778-785; Sander et al. (2011) Nature Methods 8:67-69; and Wood et al. (2011) Science 333:307, each of which is hereby incorporated by reference in its entirety), transcription activator-like effectors nucleases (TALEN) (see, e.g., Wood et al. (2011) Science 333:307; Boch et al. (2009) Science 326:1509-1512; Moscou and Bogdanove (2009) Science 326:1501; Christian et al. (2010) Genetics 186:757-761; Miller et al. (2011) Nat. Biotechnol. 29:143-148; Zhang et al. (2011) Nat. Biotechnol. 29:149-153; and Reyon et al. (2012) Nat. Biotechnol. 30(5): 460-465, each of which is hereby incorporated by reference in its entirety), homing endonucleases, meganucleases (see, e.g., U.S. Patent Publication No. US 2014/0121115, which is hereby incorporated by reference in its entirety), and RNA-guided nucleases (see, e.g., Makarova et al. (2018) The CRISPR Journal 1(5): 325-336; and Adli (2018) Nat. Communications 9:1911, each of which is hereby incorporated by reference in its entirety).

In certain embodiments, the transgene encodes an RNA-guided nuclease. Suitable RNA-guided nucleases include, without limitation, Class I and Class II clustered regularly interspaced short palindromic repeats (CRISPR)-associated nucleases. Class I is divided into types I, III, and IV, and includes, without limitation, type I (Cas3), type I-A (Cas8a, Cas5), type I-B (Cas8b), type I-C(Cas8c), type 1-D (Cas10d), type I-E (Cse1, Cse2), type I-F (Csy1, Csy2, Csy3), type I-U (GSU0054), type III (Cas10), type III-A (Csm2), type III-B (Cmr5), type III-C(Csx10 or Csx11), type III-D (Csx10), and type IV (Csf1). Class II is divided into types II, V, and VI, and includes, without limitation, type II (Cas9), type II-A (Csn2), type II-B (Cas4), type V (Cpf1, C2c1, C2c3), and type VI (Cas13a, Cas13b, Cas13c). RNA-guided nucleases also include naturally-occurring Class II CRISPR nucleases such as Cas9 (Type II) or Cas12a/Cpf1 (Type V), as well as other nucleases derived or obtained therefrom. Exemplary Cas9 nucleases that may be used in the present invention include, but are not limited to, S. pyogenes Cas9 (SpCas9), S. aureus Cas9 (SaCas9), N. meningitidis Cas9 (NmCas9), C. jejuni Cas9 (CjCas9), and Geobacillus Cas9 (GeoCas9).

In certain embodiments, the transgene encodes one or more reporter sequences, which upon expression produce a detectable signal. Such reporter sequences include, without limitation, DNA sequences encoding β-lactamase, β-galactosidase (LacZ), alkaline phosphatase, thymidine kinase, green fluorescent protein (GFP), red fluorescent protein (RFP), chloramphenicol acetyltransferase (CAT), luciferase, membrane bound proteins, including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

In certain embodiments, the rAAV genome comprises a transcriptional regulatory element (TRE) operably linked to the transgene, to control expression of an RNA or polypeptide encoded by the transgene. In certain embodiments, the TRE comprises a constitutive promoter. In certain embodiments, the TRE can be active in any mammalian cell (e.g., any human cell). In certain embodiments, the TRE is active in abroad range of human cells. Such TREs may comprise constitutive promoter and/or enhancer elements, including any of those described herein, and any of those known to one of skill in the art. In certain embodiments, the TRE comprises an inducible promoter. In certain embodiments, the TRE may be a tissue-specific TRE, i.e., it is active in specific tissue(s) and/or organ(s). A tissue-specific TRE comprises one or more tissue-specific promoter and/or enhancer elements, and optionally one or more constitutive promoter and/or enhancer elements. A skilled artisan would appreciate that tissue-specific promoter and/or enhancer elements can be isolated from genes specifically expressed in the tissue by methods well known in the art.

Suitable promoters include, e.g., cytomegalovirus promoter (CMV) (Stinski et al. (1985) Journal of Virology 55(2): 431-441), CMV early enhancer/chicken β-actin (CBA) promoter/rabbit β-globin intron (CAG) (Miyazaki et al. (1989) Gene 79(2): 269-277), CB^{SB} (Jacobson et al. (2006) Molecular Therapy 13(6): 1074-1084), human elongation factor 1α promoter (EF1α) (Kim et al. (1990) Gene 91 (2): 217-223), human phosphoglycerate kinase promoter (PGK) (Singer-Sam et al. (1984) Gene 32(3): 409-417), mitochondrial heavy-strand promoter (Lodeiro et al. (2012) PNAS 109(17): 6513-6518), ubiquitin promoter (Wulff et al. (1990) FEBS Letters 261:101-105). In certain embodiments, the TRE comprises a cytomegalovirus (CMV) promoter/enhancer (e.g., comprising a nucleotide sequence at least 90%, 91%, 92%, 93%, 94%, 95%, 96%, 97%, 98%, 99%, or 100% identical to SEQ ID NO: 18 or 19), an SV40 promoter, a chicken beta actin (CBA) promoter (e.g., comprising a nucleotide sequence at least 90%, 91%, 92%, 93%, 94%, 95%, 96%, 97%, 98%, 99%, or 100% identical to SEQ ID NO: 20 or 21), a smCBA promoter (e.g., comprising a nucleotide sequence at least 90%, 91%, 92%, 93%, 94%, 95%, 96%, 97%, 98%, 99%, or 100% identical to SEQ ID NO: 22), a human elongation factor 1 alpha (EF1α) promoter (e.g., comprising a nucleotide sequence at least 90%, 91%, 92%, 93%, 94%, 95%, 96%, 97%, 98%, 99%, or 100% identical to SEQ ID NO: 23), a minute virus of mouse (MVM) intron which comprises transcription factor binding sites (e.g., comprising a nucleotide sequence at least 90%, 91%, 92%, 93%, 94%, 95%, 96%, 97%, 98%, 99%, or 100% identical to SEQ ID NO: 24 or 25), a human phosphoglycerate kinase (PGK1) promoter, a human ubiquitin C (Ubc) promoter, a human beta actin promoter, a human neuron-specific enolase (ENO2) promoter, a human beta-glucuronidase (GUSB) promoter, a rabbit beta-globin element (e.g., comprising a nucleotide sequence at least 90%, 91%, 92%, 93%, 94%, 95%, 96%, 97%, 98%, 99%, or 100% identical to SEQ ID NO: 26 or 27), a human calmodulin 1 (CALM1) promoter (e.g., comprising a nucleotide sequence at least 90%, 91%, 92%, 93%, 94%, 95%, 96%, 97%, 98%, 99%, or 100% identical to SEQ ID NO: 28), a human ApoE/C-I hepatic control region (HCR1) (e.g., comprising a nucleotide sequence at least 90%, 91%, 92%, 93%, 94%, 95%, 96%, 97%, 98%, 99%, or 100% identical to SEQ ID NO: 29), a human al-antitrypsin (hAAT) promoter (e.g., comprising a nucleotide sequence at least 90%, 91%, 92%, 93%, 94%, 95%, 96%, 97%, 98%, 99%, or 100% identical to SEQ ID NO: 30, 31, or 32), an extended HCR1 (e.g., comprising a nucleotide sequence at least 90%, 91%, 92%, 93%, 94%, 95%, 96%, 97%, 98%, 99%, or 100% identical to SEQ ID NO: 33), an HS-CRM8 element of an hAAT promoter (e.g., comprising a nucleotide sequence at least 90%, 91%, 92%, 93%, 94%, 95%, 96%, 97%, 98%, 99%, or 100% identical to SEQ ID NO: 34), a human transthyretin (TTR) promoter (e.g., comprising a nucleotide sequence at least 90%, 91%, 92%, 93%, 94%, 95%, 96%, 97%, 98%, 99%, or 100% identical to SEQ ID NO: 35), and/or a human Methyl-CpG Binding Protein 2 (MeCP2) promoter. Any of the TREs described herein can be combined in any order to drive efficient transcription. For example, an rAAV genome may comprise a TRE comprising a CMV enhancer, a CBA promoter, and the splice acceptor from exon 3 of the rabbit beta-globin gene, collectively called a CAG promoter (e.g., comprising a nucleotide sequence at least 90%, 91%, 92%, 93%, 94%, 95%, 96%, 97%, 98%, 99%, or 100% identical to SEQ ID NO: 36). For example, an rAAV genome may comprise a TRE comprising a hybrid of CMV enhancer and CBA promoter followed by a splice donor and splice acceptor, collectively called a CASI promoter region (e.g., comprising a nucleotide sequence at least 90%, 91%, 92%, 93%, 94%, 95%, 96%, 97%, 98%, 99%, or 100% identical to SEQ ID NO: 37). For example, an rAAV genome may comprise a TRE comprising an HCR1 and hAAT promoter (also referred to as an LP1 promoter, e.g., comprising a nucleotide sequence at least 90%, 91%, 92%, 93%, 94%, 95%, 96%, 97%, 98%, 99%, or 100% identical to SEQ ID NO: 38).

In certain embodiments, the TRE is brain-specific (e.g., neuron-specific, glial cell-specific, astrocyte-specific, oligodendrocyte-specific, microglia-specific and/or central nervous system-specific). Exemplary brain-specific TREs may comprise one or more elements from, without limitation, human glial fibrillary acidic protein (GFAP) promoter, human synapsin 1 (SYN1) promoter, human synapsin 2 (SYN2) promoter, human metallothionein 3 (MT3) promoter, and/or human proteolipid protein 1 (PLP1) promoter. More brain-specific promoter elements are disclosed in WO 2016/100575A1, which is incorporated by reference herein in its entirety.

In certain embodiments, the native promoter for the transgene may be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In certain embodiments, the rAAV genome comprises an editing genome. Editing genomes can be used to edit the genome of a cell by homologous recombination of the editing genome with a genomic region surrounding a target locus in the cell. In certain embodiments, the editing genome is designed to correct a genetic defect in a gene by homologous recombination. Editing genomes generally comprise: (i) an editing element for editing a target locus in a target gene, (ii) a 5′ homology arm nucleotide sequence 5′ of the editing element having homology to a first genomic region 5′ to the target locus, and (iii) a 3′ homology arm nucleotide sequence 3′ of the editing element having homology to a second genomic region 3′ to the target locus, wherein the portion of the editing genome comprising the 5′ homology arm, editing element, and 3′ homology arm can be in the sense or antisense orientation relative to the target locus. Suitable target genes for editing using an editing genome include, without limitation, phenylalanine hydroxylase (PAH), cystic fibrosis conductance transmembrane regulator (CFTR), beta hemoglobin (HBB), oculocutaneous albinism II (OCA2), Huntingtin (HTT), dystrophia myotonica-protein kinase (DMPK), low-density lipoprotein receptor (LDLR), apolipoprotein B (APOB), neurofibromin 1 (NF1), polycystic kidney disease 1 (PKD1), polycystic kidney disease 2 (PKD2), coagulation factor VIII (F8), dystrophin (DMD), phosphate-regulating endopeptidase homologue, X-linked (PHEX), methyl-CpG-binding protein 2 (MECP2), and ubiquitin-specific peptidase 9Y, Y-linked (USP9Y). In certain embodiments, suitable target genes for editing using an editing genome are not selected from the group consisting of phenylalanine hydroxylase, arylsulfatase A, and iduronate 2-sulfatase.

In certain embodiments, the rAAV genomes disclosed herein further comprise a transcription terminator (e.g., a polyadenylation sequence). In certain embodiments, the transcription terminator is 3′ to the transgene. The transcription terminator may be any sequence that effectively terminates transcription, and a skilled artisan would appreciate that such sequences can be isolated from any genes that are expressed in the cell in which transcription of the at least a portion of an antibody coding sequence is desired. In certain embodiments, the transcription terminator comprises a polyadenylation sequence. In certain embodiments, the polyadenylation sequence is identical or substantially identical to the endogenous polyadenylation sequence of an immunoglobulin gene. In certain embodiments, the polyadenylation sequence is an exogenous polyadenylation sequence. In certain embodiments, the polyadenylation sequence is an SV40 polyadenylation sequence (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 14, 47, or 48, or a nucleotide sequence complementary thereto). In certain embodiments, the polyadenylation sequence comprises the nucleotide sequence set forth in SEQ ID NO: 14. In certain embodiments, the polyadenylation sequence consists of the nucleotide sequence set forth in SEQ ID NO: 14. In certain embodiments, the polyadenylation sequence is a bovine growth hormone (BGH) polyadenylation sequence (e.g., comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 49, or a nucleotide sequence complementary thereto). In certain embodiments, the polyadenylation sequence comprises the nucleotide sequence set forth in SEQ ID NO: 49. In certain embodiments, the polyadenylation sequence consists of the nucleotide sequence set forth in SEQ ID NO: 49.

In certain embodiments, an rAAV genome comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, or 54. In certain embodiments, the editing element comprises the nucleotide sequence set forth in SEQ ID NO: 50, 51, 52, 53, or 54. In certain embodiments, the editing element consists of the nucleotide sequence set forth in SEQ ID NO: 50, 51, 52, 53, or 54.

In certain embodiments, the rAAV genomes disclosed herein further comprise a 5′ inverted terminal repeat (5′ ITR) nucleotide sequence 5′ of the TRE, and a 3′ inverted terminal repeat (3′ ITR) nucleotide sequence 3′ of the polyadenylation sequence associated with an antibody light chain coding sequence. ITR sequences from any AAV serotype or variant thereof can be used in the rAAV genomes disclosed herein. The 5′ and 3′ ITR can be from an AAV of the same serotype or from AAVs of different serotypes. Exemplary ITRs for use in the rAAV genomes disclosed herein are set forth in SEQ ID NOs: 39, 40, 41, 42, 43, and 44, herein.

In certain embodiments, the 5′ ITR or 3′ ITR is from AAV2. In certain embodiments, both the 5′ ITR and the 3′ ITR are from AAV2. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 39, or the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 40. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 39, and the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 40. In certain embodiments, the rAAV genome comprises a 5′ ITR nucleotide sequence having the sequence of SEQ ID NO: 39, and a 3′ ITR nucleotide sequence having the sequence of SEQ ID NO: 40.

In certain embodiments, the 5′ ITR or 3′ ITR are from AAV5. In certain embodiments, both the 5′ ITR and 3′ ITR are from AAV5. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 42, or the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 43. In certain embodiments, the 5′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 42, and the 3′ ITR nucleotide sequence has at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to SEQ ID NO: 43. In certain embodiments, the rAAV genome comprises a 5′ ITR nucleotide sequence having the sequence of SEQ ID NO: 42, and a 3′ ITR nucleotide sequence having the sequence of SEQ ID NO: 43.

In certain embodiments, the 5′ ITR nucleotide sequence and the 3′ ITR nucleotide sequence are substantially complementary to each other (e.g., are complementary to each other except for mismatch at 1, 2, 3, 4, or 5 nucleotide positions in the 5′ or 3′ ITR).

In certain embodiments, the 5′ ITR or the 3′ ITR is modified to reduce or abolish resolution by Rep protein (“non-resolvable ITR”). In certain embodiments, the non-resolvable ITR comprises an insertion, deletion, or substitution in the nucleotide sequence of the terminal resolution site. Such modification allows formation of a self-complementary, double-stranded DNA genome of the AAV after the rAAV genome is replicated in an infected cell. Exemplary non-resolvable ITR sequences are known in the art (see, e.g., those provided in U.S. Pat. Nos. 7,790,154 and 9,783,824, which are incorporated by reference herein in their entirety). In certain embodiments, the 5′ ITR comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 41. In certain embodiments, the 5′ ITR consists of a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 41. In certain embodiments, the 5′ ITR consists of the nucleotide sequence set forth in SEQ ID NO: 41. In certain embodiments, the 3′ ITR comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 44. In certain embodiments, the 5′ ITR consists of a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 44. In certain embodiments, the 3′ ITR consists of the nucleotide sequence set forth in SEQ ID NO: 44. In certain embodiments, the 5′ ITR consists of the nucleotide sequence set forth in SEQ ID NO: 41, and the 3′ ITR consists of the nucleotide sequence set forth in SEQ ID NO: 44. In certain embodiments, the 5′ ITR consists of the nucleotide sequence set forth in SEQ ID NO: 41, and the 3′ ITR consists of the nucleotide sequence set forth in SEQ ID NO: 44.

In certain embodiments, the 5′ ITR is flanked by an additional nucleotide sequence derived from a wild-type AAV2 genomic sequence. In certain embodiments, the 5′ ITR is flanked by an additional 46 bp sequence derived from a wild-type AAV2 sequence that is adjacent to a wild-type AAV2 ITR in an AAV2 genome. In certain embodiments, the additional 46 bp sequence is 3′ to the 5′ ITR in the rAAV genome. In certain embodiments, the 46 bp sequence consists of the nucleotide sequence set forth in SEQ ID NO: 45.

In certain embodiments, the 3′ ITR is flanked by an additional nucleotide sequence derived from a wild-type AAV2 genomic sequence. In certain embodiments, the 3′ ITR is flanked by an additional 37 bp sequence derived from a wild-type AAV2 sequence that is adjacent to a wild-type AAV2 ITR in an AAV2 genome. See, e.g., Savy et al., Human Gene Therapy Methods (2017) 28(5): 277-289 (which is hereby incorporated by reference herein in its entirety). In certain embodiments, the additional 37 bp sequence is 5′ to the 3′ ITR in the rAAV genome. In certain embodiments, the 37 bp sequence consists of the nucleotide sequence set forth in SEQ ID NO: 46.

In certain embodiments, an rAAV genome comprises a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 55, 56, 57, 58, or 59. In certain embodiments, the editing element comprises the nucleotide sequence set forth in SEQ ID NO: 55, 56, 57, 58, or 59. In certain embodiments, the editing element consists of the nucleotide sequence set forth in SEQ ID NO: 55, 56, 57, 58, or 59.

### AAV Capsid Protein

In certain embodiments, the pharmaceutical compositions of the present disclosure comprise an rAAV comprising an AAV capsid. In certain embodiments, the AAV capsid comprises an AAV capsid protein. The rAAV can comprise an AAV capsid comprising an AAV capsid protein from any AAV capsid known in the art, including natural AAV isolates and variants thereof.

AAV capsid proteins include VP1, VP2, and VP3 capsid proteins. VP1, VP2, and/or VP3 capsid proteins assemble into a capsid that surrounds the rAAV genome. In certain embodiments, assembly of the capsid proteins is facilitated by the assembly-activating protein (AAP). Capsids of certain AAV serotypes require the role of AAP in transporting the capsid proteins to the nucleolus for assembly. For example, AAV1, AAV2, AAV3, AAV6, AAV7, AAV8, AAV9, AAV10, and AAV12 require AAP to form capsids, while capsids of AAV4, AAV5, and AAV11 can assemble without AAP. See, e.g., Earley et al. (2017) J. Virol. 91(3): e01980-16.

Different AAV serotypes or variants thereof comprise AAV capsid proteins having different amino acid sequences. Suitable AAV capsid proteins include, without limitation, a capsid protein from AAV1, AAV2, AAV3, AAV4, AAV5, AAV6, AAV7, AAV8, AAV9, AAV10, AAV11, AAV12, AAV13, AAV-DJ, AAV-LK03, NP59, VOY101, VOY201, VOY701, VOY801, VOY1101, AAVPHP.N, AAVPHP.A, AAVPHP.B, PHP.B2, PHP.B3, G2A3, G2B4, G2B5, PHP.S, AAVRh32.33, AAVrh74, AAVrh10, AAVHSC1, AAVHSC2, AAVHSC3, AAVHSC4, AAVHSC5, AAVHSC6, AAVHSC7, AAVHSC8, AAVHSC9, AAVHSC10, AAVHSC11, AAVHSC12, AAVHSC13, AAVHSC14, AAVHSC15, AAVHSC16, AAVHSC17, and any variants thereof. In certain embodiments, the AAV capsid protein is not from an AAVHSC. The sequences of the various AAV capsid proteins are disclosed in, e.g., U.S. Patent Publication Nos.: US20140359799, US20150376607, US20150159173, US20170081680, and US20170360962A1, and PCT Publication No. WO2020227515, the disclosures of which are incorporated by reference herein in their entireties.

For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 203-736 of SEQ ID NO: 1, 2, 3, 4, 5, 6, 7, 8, 9, 10, 11, 12, 13, 15, 16, or 17. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 203-736 of SEQ ID NO: 1, 2, 3, 4, 5, 6, 7, 8, 9, 10, 11, 12, 13, 15, 16, or 17, wherein: the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the amino acid in the capsid protein corresponding to amino acid 626 of SEQ ID NO: 16 is G, and the amino acid in the capsid protein corresponding to amino acid 718 of SEQ ID NO: 16 is G. In certain embodiments, the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M. In certain embodiments, the amino acid in the capsid protein corresponding to amino acid 505 of SEQ ID NO: 16 is R, and the amino acid in the capsid protein corresponding to amino acid 687 of SEQ ID NO: 16 is R. In certain embodiments, the amino acid in the capsid protein corresponding to amino acid 346 of SEQ ID NO: 16 is A, and the amino acid in the capsid protein corresponding to amino acid 505 of SEQ ID NO: 16 is R. In certain embodiments, the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the capsid protein comprises the amino acid sequence of amino acids 203-736 of SEQ ID NO: 1, 2, 3, 4, 5, 6, 7, 8, 9, 10, 11, 12, 13, 15, 16, or 17.

For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 138-736 of SEQ ID NO: 1, 2, 3, 4, 5, 6, 7, 8, 9, 10, 11, 12, 13, 15, 16, or 17. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 138-736 of SEQ ID NO: 1, 2, 3, 4, 5, 6, 7, 8, 9, 10, 11, 12, 13, 15, 16, or 17, wherein: the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the amino acid in the capsid protein corresponding to amino acid 626 of SEQ ID NO: 16 is G, and the amino acid in the capsid protein corresponding to amino acid 718 of SEQ ID NO: 16 is G. In certain embodiments, the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M. In certain embodiments, the amino acid in the capsid protein corresponding to amino acid 505 of SEQ ID NO: 16 is R, and the amino acid in the capsid protein corresponding to amino acid 687 of SEQ ID NO: 16 is R. In certain embodiments, the amino acid in the capsid protein corresponding to amino acid 346 of SEQ ID NO: 16 is A, and the amino acid in the capsid protein corresponding to amino acid 505 of SEQ ID NO: 16 is R. In certain embodiments, the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the capsid protein comprises the amino acid sequence of amino acids 138-736 of SEQ ID NO: 1, 2, 3, 4, 5, 6, 7, 8, 9, 10, 11, 12, 13, 15, 16, or 17.

For example, in certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 1-736 of SEQ ID NO: 1, 2, 3, 4, 5, 6, 7, 8, 9, 10, 11, 12, 13, 15, 16, or 17. In certain embodiments, the capsid protein comprises an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the amino acid sequence of amino acids 1-736 of SEQ ID NO: 1, 2, 3, 4, 5, 6, 7, 8, 9, 10, 11, 12, 13, 15, 16, or 17, wherein: the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G. In certain embodiments, the amino acid in the capsid protein corresponding to amino acid 2 of SEQ ID NO: 16 is T, and the amino acid in the capsid protein corresponding to amino acid 312 of SEQ ID NO: 16 is Q. In certain embodiments, the amino acid in the capsid protein corresponding to amino acid 65 of SEQ ID NO: 16 is I, and the amino acid in the capsid protein corresponding to amino acid 626 of SEQ ID NO: 16 is Y. In certain embodiments, the amino acid in the capsid protein corresponding to amino acid 77 of SEQ ID NO: 16 is R, and the amino acid in the capsid protein corresponding to amino acid 690 of SEQ ID NO: 16 is K. In certain embodiments, the amino acid in the capsid protein corresponding to amino acid 119 of SEQ ID NO: 16 is L, and the amino acid in the capsid protein corresponding to amino acid 468 of SEQ ID NO: 16 is S. In certain embodiments, the amino acid in the capsid protein corresponding to amino acid 626 of SEQ ID NO: 16 is G, and the amino acid in the capsid protein corresponding to amino acid 718 of SEQ ID NO: 16 is G. In certain embodiments, the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M. In certain embodiments, the amino acid in the capsid protein corresponding to amino acid 505 of SEQ ID NO: 16 is R, and the amino acid in the capsid protein corresponding to amino acid 687 of SEQ ID NO: 16 is R. In certain embodiments, the amino acid in the capsid protein corresponding to amino acid 346 of SEQ ID NO: 16 is A, and the amino acid in the capsid protein corresponding to amino acid 505 of SEQ ID NO: 16 is R. In certain embodiments, the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C. In certain embodiments, the capsid protein comprises the amino acid sequence of amino acids 1-736 of SEQ ID NO: 1, 2, 3, 4, 5, 6, 7, 8, 9, 10, 11, 12, 13, 15, 16, or 17.

In certain embodiments, the AAV capsid comprises two or more of: (a) a capsid protein comprising the amino acid sequence of amino acids 203-736 of SEQ ID NO: 1, 2, 3, 4, 6, 7, 10, 11, 12, 13, 15, 16, or 17; (b) a capsid protein comprising the amino acid sequence of amino acids 138-736 of SEQ ID NO: 1, 2, 3, 4, 5, 6, 7, 9, 10, 11, 12, 13, 15, 16, or 17; and (c) a capsid protein comprising the amino acid sequence of amino acids 1-736 of SEQ ID NO: 1, 2, 3, 4, 5, 6, 7, 8, 9, 10, 11, 12, 13, 15, 16, or 17. In certain embodiments, the AAV capsid comprises: (a) a capsid protein having an amino acid sequence consisting of amino acids 203-736 of SEQ ID NO: 1, 2, 3, 4, 6, 7, 10, 11, 12, 13, 15, 16, or 17; (b) a capsid protein having an amino acid sequence consisting of amino acids 138-736 of SEQ ID NO: 1, 2, 3, 4, 5, 6, 7, 9, 10, 11, 12, 13, 15, 16, or 17; and (c) a capsid protein having an amino acid sequence consisting of amino acids 1-736 of SEQ ID NO: 1, 2, 3, 4, 5, 6, 7, 8, 9, 10, 11, 12, 13, 15, 16, or 17.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 203-736 of SEQ ID NO: 8; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 138-736 of SEQ ID NO: 8; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 1-736 of SEQ ID NO: 8. In certain embodiments, the AAV capsid comprises one or more of: (a) a capsid protein comprising the amino acid sequence of amino acids 203-736 of SEQ ID NO: 8; (b) a capsid protein comprising the amino acid sequence of amino acids 138-736 of SEQ ID NO: 8; and (c) a capsid protein comprising the amino acid sequence of amino acids 1-736 of SEQ ID NO: 8. In certain embodiments, the AAV capsid comprises two or more of: (a) a capsid protein comprising the amino acid sequence of amino acids 203-736 of SEQ ID NO: 8; (b) a capsid protein comprising the amino acid sequence of amino acids 138-736 of SEQ ID NO: 8; and (c) a capsid protein comprising the amino acid sequence of amino acids 1-736 of SEQ ID NO: 8. In certain embodiments, the AAV capsid comprises: (a) a capsid protein having an amino acid sequence consisting of amino acids 203-736 of SEQ ID NO: 8; (b) a capsid protein having an amino acid sequence consisting of amino acids 138-736 of SEQ ID NO: 8; and (c) a capsid protein having an amino acid sequence consisting of amino acids 1-736 of SEQ ID NO: 8.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 203-736 of SEQ ID NO: 11; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 138-736 of SEQ ID NO: 11; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 1-736 of SEQ ID NO: 11. In certain embodiments, the AAV capsid comprises one or more of: (a) a capsid protein comprising the amino acid sequence of amino acids 203-736 of SEQ ID NO: 11; (b) a capsid protein comprising the amino acid sequence of amino acids 138-736 of SEQ ID NO: 11; and (c) a capsid protein comprising the amino acid sequence of amino acids 1-736 of SEQ ID NO: 11. In certain embodiments, the AAV capsid comprises two or more of: (a) a capsid protein comprising the amino acid sequence of amino acids 203-736 of SEQ ID NO: 11; (b) a capsid protein comprising the amino acid sequence of amino acids 138-736 of SEQ ID NO: 11; and (c) a capsid protein comprising the amino acid sequence of amino acids 1-736 of SEQ ID NO: 11. In certain embodiments, the AAV capsid comprises: (a) a capsid protein having an amino acid sequence consisting of amino acids 203-736 of SEQ ID NO: 11; (b) a capsid protein having an amino acid sequence consisting of amino acids 138-736 of SEQ ID NO: 11; and (c) a capsid protein having an amino acid sequence consisting of amino acids 1-736 of SEQ ID NO: 11.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 203-736 of SEQ ID NO: 13; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 138-736 of SEQ ID NO: 13; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the sequence of amino acids 1-736 of SEQ ID NO: 13. In certain embodiments, the AAV capsid comprises one or more of: (a) a capsid protein comprising the amino acid sequence of amino acids 203-736 of SEQ ID NO: 13; (b) a capsid protein comprising the amino acid sequence of amino acids 138-736 of SEQ ID NO: 13; and (c) a capsid protein comprising the amino acid sequence of amino acids 1-736 of SEQ ID NO: 13. In certain embodiments, the AAV capsid comprises two or more of: (a) a capsid protein comprising the amino acid sequence of amino acids 203-736 of SEQ ID NO: 13; (b) a capsid protein comprising the amino acid sequence of amino acids 138-736 of SEQ ID NO: 13; and (c) a capsid protein comprising the amino acid sequence of amino acids 1-736 of SEQ ID NO: 13. In certain embodiments, the AAV capsid comprises: (a) a capsid protein having an amino acid sequence consisting of amino acids 203-736 of SEQ ID NO: 13; (b) a capsid protein having an amino acid sequence consisting of amino acids 138-736 of SEQ ID NO: 13; and (c) a capsid protein having an amino acid sequence consisting of amino acids 1-736 of SEQ ID NO: 13.

In certain embodiments, the AAV capsid comprises one or more of: (a) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 203-736 of SEQ ID NO: 16; (b) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 138-736 of SEQ ID NO: 16; and (c) a capsid protein comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity with the sequence of amino acids 1-736 of SEQ ID NO: 16. In certain embodiments, the AAV capsid comprises one or more of: (a) a capsid protein comprising the amino acid sequence of amino acids 203-736 of SEQ ID NO: 16; (b) a capsid protein comprising the amino acid sequence of amino acids 138-736 of SEQ ID NO: 16; and (c) a capsid protein comprising the amino acid sequence of amino acids 1-736 of SEQ ID NO: 16. In certain embodiments, the AAV capsid comprises two or more of: (a) a capsid protein comprising the amino acid sequence of amino acids 203-736 of SEQ ID NO: 16; (b) a capsid protein comprising the amino acid sequence of amino acids 138-736 of SEQ ID NO: 16; and (c) a capsid protein comprising the amino acid sequence of amino acids 1-736 of SEQ ID NO: 16. In certain embodiments, the AAV capsid comprises: (a) a capsid protein having an amino acid sequence consisting of amino acids 203-736 of SEQ ID NO: 16; (b) a capsid protein having an amino acid sequence consisting of amino acids 138-736 of SEQ ID NO: 16; and (c) a capsid protein having an amino acid sequence consisting of amino acids 1-736 of SEQ ID NO: 16.

### III. Excipients and pH

The pharmaceutical compositions provided by the present disclosure comprise one or more excipients. As used herein, the term “excipient,” refers to any non-therapeutic agent added to the pharmaceutical composition (e.g., formulation) to provide certain desired characteristics, e.g., a desired pH, viscosity, or stabilizing effect.

In certain embodiments, a pharmaceutical composition of the present disclosure comprises one or more excipients in an amount that stabilizes the AAV (e.g., rAAV). AAV stabilization can refer to, without limitation, maintained viral titers, maintained AAV vector purity (e.g., improved control over the levels of aggregates, oxidized species, and other degradation products), maintained potency, and reduced AAV degradation (e.g., maintained retention of capsid proteins) over time and at various temperatures (e.g., various storage temperatures).

In certain embodiments, the one or more excipients in the pharmaceutical composition are capable of controlling the level of aggregates (e.g., reduce the accumulation of aggregates). AAV aggregates can reduce viral titer and product efficacy, and at the same time, can increase immunogenicity. Aggregation of AAV vectors may be formed at multiple points in the manufacturing and purification processes and during subsequent formulation and storage. For example, aggregation has been found to occur following freeze-thaw cycles and during concentration of purified rAAV, see, e.g., Wright et al., Mol. Ther. (2005) 12(1): 171-178.

In certain embodiments, the pharmaceutical composition comprises a buffering agent or pH adjusting agent. In certain embodiments, the pharmaceutical composition comprises about 1 mM to about 50 mM of the buffering agent, for example, about 1 mM to about 10 mM, about 5 mM to about 15 mM, about 10 mM to about 20 mM, about 15 mM to about 25 mM, about 20 mM to about 30 mM, about 25 mM to about 35 mM, about 30 mM to about 40 mM, about 35 mM to about 45 mM, or about 40 mM to about 50 mM of the buffering agent. In certain embodiments, the pharmaceutical composition comprises about 1 mM, about 5 mM, about 10 mM, about 15 mM, about 20 mM, about 25 mM, about 30 mM, about 35 mM, about 40 mM, about 45 mM, or about 50 mM of the buffering agent. In certain embodiments, the pharmaceutical composition comprises about 20 mM of the buffering agent.

Pharmaceutically acceptable buffering agents are well known in the art, including, without limitation, sodium phosphate dibasic, potassium phosphate monobasic, potassium phosphate, potassium chloride, sodium chloride, sodium phosphate dibasic anhydrous, sodium phosphate hexahydrate, sodium phosphate monobasic monohydrate, sodium phosphate, sodium succinate, sodium citrate, sodium acetate, sodium carbonate, sodium sulfate, magnesium sulfate, magnesium chloride, tromethamine, tris(hydroxymethyl)aminomethane hydrochloride (Tris-HCl), histidine (e.g., L-histidine), histidine hydrochloride (histidine-HCl), bicine, glycine, glycyl glycine, lysine, arginine, 4-(2-hydroxyethyl)-1-piperazineethanesulfonic acid (HEPES), calcium sulfate, calcium chloride, calcium citrate, and any mixtures thereof.

In certain embodiments, the buffering agent is sodium phosphate dibasic or sodium phosphate. In certain embodiments, the pharmaceutical composition comprises sodium phosphate dibasic or sodium phosphate. Accordingly, in certain embodiments, the pharmaceutical composition comprises about 1 mM to about 50 mM sodium phosphate dibasic or sodium phosphate, for example, about 1 mM to about 10 mM, about 5 mM to about 15 mM, about 10 mM to about 20 mM, about 15 mM to about 25 mM, about 20 mM to about 30 mM, about 25 mM to about 35 mM, about 30 mM to about 40 mM, about 35 mM to about 45 mM, or about 40 mM to about 50 mM sodium phosphate dibasic or sodium phosphate. In certain embodiments, the pharmaceutical composition comprises about 1 mM, 5 mM, about 10 mM, about 15 mM, about 20 mM, about 25 mM, about 30 mM, about 35 mM, about 40 mM, about 45 mM, or about 50 mM sodium phosphate dibasic or sodium phosphate. In certain embodiments, the pharmaceutical composition comprises about 20 mM sodium phosphate.

In certain embodiments, the buffering agent is histidine. In certain embodiments, the pharmaceutical composition comprises histidine. Accordingly, in certain embodiments, the pharmaceutical composition comprises about 1 mM to about 50 mM histidine, for example, about 1 mM to about 10 mM, about 5 mM to about 15 mM, about 10 mM to about 20 mM, about 15 mM to about 25 mM, about 20 mM to about 30 mM, about 25 mM to about 35 mM, about 30 mM to about 40 mM, about 35 mM to about 45 mM, or about 40 mM to about 50 mM histidine. In certain embodiments, the pharmaceutical composition comprises about 1 mM, about 5 mM, about 10 mM, about 15 mM, about 20 mM, about 25 mM, about 30 mM, about 35 mM, about 40 mM, about 45 mM, or about 50 mM histidine. In certain embodiments, the pharmaceutical composition comprises about 20 mM histidine.

In certain embodiments, pharmaceutical compositions comprising histidine as described herein provide increased long-term stability of AAV titer (e.g., rAAV titer). The increased stability of AAV titer (e.g., rAAV titer) provided by the pharmaceutical compositions comprising histidine was found to extend across various thermal stresses (e.g., extend across studies performed at −80° C., 2-8° C., 5° C., 25° C., and 40° C.). Various methods of determining AAV titer within a given pharmaceutical composition are well known in the art. For example, droplet digital polymerase chain reaction (ddPCR) can be used to titer AAV in a given composition.

In certain embodiments, pharmaceutical compositions comprising histidine as described herein provide increased long-term purity of AAV (e.g., rAAV) vectors. The increased long-term purity of AAV (e.g., rAAV) vectors provided by the pharmaceutical compositions comprising histidine was found to extend across various thermal stresses (e.g., extend across studies performed at −80° C., 2-8° C., 5° C., 25° C., and 40° C.). In certain embodiments, pharmaceutical compositions comprising histidine as described herein provide increased long-term retention of the VP1 capsid protein, which is a key factor in capsid potency. The increased long-term retention of the VP1 capsid protein provided by the pharmaceutical compositions comprising histidine was found to extend across various thermal stresses (e.g., extend across studies performed at −80° C., 2-8° C., 5° C., 25° C., and 40° C.). Various methods of determining AAV vector purity and the level of VP1 capsid protein retention within a given pharmaceutical composition are well known in the art. For example, capillary electrophoresis sodium dodecyl sulfate (CE-SDS) can be performed to determine the purity of AAV vectors and the level of VP1 capsid protein retention in a given composition.

In certain embodiments, pharmaceutical compositions comprising histidine as described herein provide improved control over the level of aggregates (e.g., reduce the accumulation of aggregates) over time. The improved control over the level of aggregates over time provided by the pharmaceutical compositions comprising histidine were found to extend across various thermal stresses (e.g., extend across studies performed at −80° C., 2-8° C., 5° C., 25° C., and 40° C.). Various methods of determining the level of aggregation within a given pharmaceutical composition are well known in the art. For example, size exclusion chromatography (SEC) analysis can be used to determine the level of aggregation in a given composition.

In certain embodiments, pharmaceutical compositions comprising histidine as described herein provide increased long-term potency of AAV (e.g., rAAV) vectors. The increased long-term potency of AAV (e.g., rAAV) vectors provided by the pharmaceutical compositions comprising histidine was found to extend across various thermal stresses (e.g., extend across studies performed at −80° C., 2-8° C., 5° C., 25° C., and 40° C.). In certain embodiments, pharmaceutical compositions comprising histidine as described herein provide improved control over the relative gene expression (% RGE), which is a key factor in AAV vector potency.

In certain embodiments, the pharmaceutical composition comprises a salt. In certain embodiments, the pharmaceutical composition comprises about 50 mM to about 200 mM of the salt, for example, about 50 mM to about 90 mM, about 70 mM to about 110 mM, about 90 mM to about 130 mM, about 110 mM to about 150 mM, about 130 mM to about 170 mM, about 150 mM to about 190 mM, about 170 mM to about 200 mM, or about 150 mM to about 200 mM of the salt. In certain embodiments, the pharmaceutical composition comprises about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, or about 200 mM of the salt. In certain embodiments, the pharmaceutical composition comprises greater than about 150 mM of the salt. In certain embodiments, the pharmaceutical composition comprises about 150 mM of the salt. In certain embodiments, the pharmaceutical composition comprises about 175 mM of the salt. In certain embodiments, the pharmaceutical composition comprises about 200 mM of the salt.

Pharmaceutically acceptable salts are well known in the art, including, without limitation, potassium chloride, sodium chloride, magnesium chloride, calcium chloride, and any mixtures thereof. In certain embodiments, the salt is a monovalent salt.

In certain embodiments, the pharmaceutical composition comprises a monovalent salt. In certain embodiments, the salt is a monovalent salt having an ionic strength of about 50 mM to about 200 mM, for example, about 50 mM to about 90 mM, about 70 mM to about 110 mM, about 90 mM to about 130 mM, about 110 mM to about 150 mM, about 130 mM to about 170 mM, about 150 mM to about 190 mM, about 170 mM to about 200 mM, or about 150 mM to about 200 mM. In certain embodiments, the pharmaceutical composition comprises a monovalent salt having an ionic strength of about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, or about 200 mM. In certain embodiments, the pharmaceutical composition comprises a monovalent salt having an ionic strength of greater than about 150 mM. In certain embodiments, the pharmaceutical composition comprises a monovalent salt having an ionic strength of about 150 mM. In certain embodiments, the pharmaceutical composition comprises a monovalent salt having an ionic strength of about 175 mM. In certain embodiments, the pharmaceutical composition comprises a monovalent salt having an ionic strength of about 200 mM.

Where reference is made to a specific concentration of sodium chloride or potassium chloride, or a range of concentrations thereto, the skilled artisan readily appreciates that the sodium chloride or potassium chloride can be substituted for another salt, or a mixture of suitable salts, adjusted to the appropriate concentration to provide an ionic strength equivalent to that provided by the sodium chloride or potassium chloride. As such, salt concentrations referred to herein may also refer to an equivalent ionic strength provided by the salt concentration, and may be referred to as “or an ionic strength equivalent thereto.”

In certain embodiments, the salt is sodium chloride. In certain embodiments, the pharmaceutical composition comprises sodium chloride. Accordingly, in certain embodiments, the pharmaceutical composition comprises about 50 mM to about 200 mM sodium chloride or an ionic strength equivalent thereto, for example, about 50 mM to about 90 mM, about 70 mM to about 110 mM, about 90 mM to about 130 mM, about 110 mM to about 150 mM, about 130 mM to about 170 mM, about 150 mM to about 190 mM, about 170 mM to about 200 mM, or about 150 mM to about 200 mM sodium chloride or an ionic strength equivalent thereto. In certain embodiments, the pharmaceutical composition comprises about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, or about 200 mM sodium chloride or an ionic strength equivalent thereto. In certain embodiments, the pharmaceutical composition comprises greater than about 150 mM sodium chloride or an ionic strength equivalent thereto. In certain embodiments, the pharmaceutical composition comprises about 150 mM sodium chloride or an ionic strength equivalent thereto. In certain embodiments, the pharmaceutical composition comprises about 175 mM sodium chloride or an ionic strength equivalent thereto. In certain embodiments, the pharmaceutical composition comprises about 200 mM sodium chloride or an ionic strength equivalent thereto.

In certain embodiments, the salt is potassium chloride. In certain embodiments, the pharmaceutical composition comprises potassium chloride. Accordingly, in certain embodiments, the pharmaceutical composition comprises about 50 mM to about 200 mM potassium chloride or an ionic strength equivalent thereto, for example, about 50 mM to about 90 mM, about 70 mM to about 110 mM, about 90 mM to about 130 mM, about 110 mM to about 150 mM, about 130 mM to about 170 mM, about 150 mM to about 190 mM, about 170 mM to about 200 mM, or about 150 mM to about 200 mM potassium chloride or an ionic strength equivalent thereto. In certain embodiments, the pharmaceutical composition comprises about 50 mM, about 60 mM, about 70 mM, about 80 mM, about 90 mM, about 100 mM, about 110 mM, about 120 mM, about 130 mM, about 140 mM, about 150 mM, about 160 mM, about 170 mM, about 180 mM, about 190 mM, or about 200 mM potassium chloride or an ionic strength equivalent thereto. In certain embodiments, the pharmaceutical composition comprises greater than about 150 mM potassium chloride or an ionic strength equivalent thereto. In certain embodiments, the pharmaceutical composition comprises about 150 mM potassium chloride or an ionic strength equivalent thereto. In certain embodiments, the pharmaceutical composition comprises about 175 mM potassium chloride or an ionic strength equivalent thereto. In certain embodiments, the pharmaceutical composition comprises about 200 mM potassium chloride or an ionic strength equivalent thereto.

In certain embodiments, the pharmaceutical composition comprises a stabilizing agent. In certain embodiments, the pharmaceutical composition comprises about 0.01% (w/v) to about 10% (w/v) of the stabilizing agent, for example, about 0.01% (w/v) to about 0.1% (w/v), about 0.050% (w/v) to about 0.50% (w/v), about 0.10% (w/v) to about 10% (w/v), 10% (w/v) to about 3% (w/v), about 2% (w/v) to about 4% (w/v), about 3% (w/v) to about 5% (w/v), about 4% (w/v) to about 6% (w/v), about 5% (w/v) to about 7% (w/v), about 6% (w/v) to about 8% (w/v), about 7% (w/v) to about 9% (w/v), or about 8% (w/v) to about 10% (w/v) of the stabilizing agent. In certain embodiments, the pharmaceutical composition comprises about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v) of the stabilizing agent. In certain embodiments, the pharmaceutical composition comprises about 1% (w/v) of the stabilizing agent. In certain embodiments, the pharmaceutical composition comprises about 3% (w/v) of the stabilizing agent. In certain embodiments, the pharmaceutical composition comprises about 5% (w/v) of the stabilizing agent.

Pharmaceutically acceptable stabilizing agents are well known in the art, including, without limitation, any sugar or sugar-alcohol such as a monosaccharide, a disaccharide, a polysaccharide, a glucan (e.g., water-soluble glucan), e.g., fructose, glucose, mannose, sorbose, sorbitol, xylose, maltose, lactose, sucrose, dextran, trehalose, pullulan, dextrin, cyclodextrin, soluble starch, hydroxyethyl starch, and carboxymethylcellulose, and any mixtures thereof.

In certain embodiments, the stabilizing agent is sucrose. In certain embodiments, the pharmaceutical composition comprises sucrose. Accordingly, in certain embodiments, the pharmaceutical composition comprises about 1% (w/v) to about 10% (w/v) sucrose, for example, about 1% (w/v) to about 3% (w/v), about 2% (w/v) to about 4% (w/v), about 3% (w/v) to about 5% (w/v), about 4% (w/v) to about 6% (w/v), about 5% (w/v) to about 7% (w/v), about 6% (w/v) to about 8% (w/v), about 7% (w/v) to about 9% (w/v), or about 8% (w/v) to about 10% (w/v) sucrose. In certain embodiments, the pharmaceutical composition comprises about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v) sucrose. In certain embodiments, the pharmaceutical composition comprises about 1% (w/v) sucrose. In certain embodiments, the pharmaceutical composition comprises about 3% (w/v) sucrose. In certain embodiments, the pharmaceutical composition comprises about 5% (w/v) sucrose.

In certain embodiments, the stabilizing agent is trehalose. In certain embodiments, the pharmaceutical composition comprises trehalose. Accordingly, in certain embodiments, the pharmaceutical composition comprises about 1% (w/v) to about 10% (w/v) trehalose, for example, about 1% (w/v) to about 3% (w/v), about 2% (w/v) to about 4% (w/v), about 3% (w/v) to about 5% (w/v), about 4% (w/v) to about 6% (w/v), about 5% (w/v) to about 7% (w/v), about 6% (w/v) to about 8% (w/v), about 7% (w/v) to about 9% (w/v), or about 8% (w/v) to about 10% (w/v) trehalose. In certain embodiments, the pharmaceutical composition comprises about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v) trehalose. In certain embodiments, the pharmaceutical composition comprises about 1% (w/v) trehalose. In certain embodiments, the pharmaceutical composition comprises about 3% (w/v) trehalose. In certain embodiments, the pharmaceutical composition comprises about 5% (w/v) trehalose.

In certain embodiments, pharmaceutical compositions comprising trehalose as described herein provide improved control over the levels of aggregation (e.g., reduce the accumulation of aggregates) over time. The improved control over the levels of aggregation over time provided by the pharmaceutical compositions comprising trehalose were found to extend across various thermal stresses (e.g., extend across studies performed at −80° C., 2-8° C., 5° C., 25° C., and 40° C.). Various methods of determining the level of aggregation within a given pharmaceutical composition are well known in the art. For example, size exclusion chromatography (SEC) analysis can be used to determine the level of aggregation in a given composition.

In certain embodiments, the pharmaceutical composition comprises a surfactant (e.g., a non-ionic surfactant). In certain embodiments, the pharmaceutical composition comprises about 0.001% (w/v) to about 1% (w/v) of the surfactant (e.g., a non-ionic surfactant), for example, about 0.001% (w/v) to about 0.05% (w/v), about 0.01% (w/v) to about 0.05% (w/v), about 0.05% (w/v) to about 0.10% (w/v), about 0.10% (w/v) to about 0.50% (w/v), or about 0.50% (w/v) to about 1% (w/v) of the surfactant (e.g., a non-ionic surfactant). In certain embodiments, the pharmaceutical composition comprises about 0.001% (w/v), about 0.005% (w/v), about 0.01% (w/v), about 0.015% (w/v), about 0.02% (w/v), about 0.025% (w/v), about 0.03% (w/v), about 0.035% (w/v), about 0.04% (w/v), about 0.045% (w/v), about 0.05% (w/v), about 0.055% (w/v), about 0.06% (w/v), about 0.065% (w/v), about 0.07% (w/v), about 0.075% (w/v), about 0.08% (w/v), about 0.085% (w/v), about 0.09% (w/v), about 0.095% (w/v), about 0.10% (w/v), about 0.2% (w/v), about 0.30% (w/v), about 0.4% (w/v), about 0.50% (w/v), about 0.6% (w/v), about 0.7% (w/v), about 0.8% (w/v), about 0.9% (w/v), or about 1% (w/v) of the surfactant (e.g., a non-ionic surfactant). In certain embodiments, the pharmaceutical composition comprises about 0.03% (w/v) of the surfactant (e.g., a non-ionic surfactant).

Pharmaceutically acceptable surfactants are well known in the art, including, without limitation, Polysorbate 20, Polysorbate 80, Brij-35, Poloxamer 188, and any mixtures thereof. In certain embodiments, the surfactant is a non-ionic surfactant. In certain embodiments, the surfactant is Poloxamer 188 (P188).

In certain embodiments, the surfactant is Poloxamer 188 (P188). In certain embodiments, the pharmaceutical composition comprises Poloxamer 188 (P188). Accordingly, in certain embodiments, the pharmaceutical composition comprises about 0.001% (w/v) to about 1% (w/v) of the surfactant, for example, about 0.001% (w/v) to about 0.05% (w/v), about 0.01% (w/v) to about 0.050% (w/v), about 0.050% (w/v) to about 0.1% (w/v), about 0.1% (w/v) to about 0.50% (w/v), or about 0.5% (w/v) to about 1% (w/v) Poloxamer 188 (P188). In certain embodiments, the pharmaceutical composition comprises about 0.001% (w/v), about 0.005% (w/v), about 0.01% (w/v), about 0.015% (w/v), about 0.02% (w/v), about 0.025% (w/v), about 0.03% (w/v), about 0.035% (w/v), about 0.04% (w/v), about 0.045% (w/v), about 0.05% (w/v), about 0.055% (w/v), about 0.06% (w/v), about 0.065% (w/v), about 0.07% (w/v), about 0.075% (w/v), about 0.08% (w/v), about 0.085% (w/v), about 0.09% (w/v), about 0.095% (w/v), about 0.1% (w/v), about 0.2% (w/v), about 0.30% (w/v), about 0.4% (w/v), about 0.5% (w/v), about 0.6% (w/v), about 0.7% (w/v), about 0.8% (w/v), about 0.9% (w/v), or about 1% (w/v) Poloxamer 188 (P188). In certain embodiments, the pharmaceutical composition comprises about 0.03% (w/v) Poloxamer 188 (P188).

The pharmaceutical compositions of the present disclosure may have a pH of about 6.0 to about 8.0, for example, about 6.0 to about 6.4, about 6.2 to about 6.6, about 6.4 to about 6.8, about 6.6 to about 7.0, about 6.8 to about 7.2, about 7.0 to about 7.4, about 7.2 to about 7.6, about 7.4 to about 7.8, or about 7.6 to about 8.0. In certain embodiments, the pharmaceutical composition has a pH of about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, or about 8.0. In certain embodiments, the pharmaceutical composition has a pH of about 7.3.

The pharmaceutical compositions of the present disclosure may have a conductivity of about 15 mS/cm to about 20 mS/cm at about 20° C., for example, about 15 mS/cm to about 16 mS/cm at about 20° C., about 16 mS/cm to about 17 mS/cm at about 20° C., about 17 mS/cm to about 18 mS/cm at about 20° C., about 18 mS/cm to about 19 mS/cm at about 20° C., about 19 mS/cm to about 20 mS/cm at about 20° C. In certain embodiments, the pharmaceutical composition has a conductivity of about 15 mS/cm at about 20° C., about 15.5 mS/cm at about 20° C., about 16 mS/cm at about 20° C., about 16.5 mS/cm at about 20° C., about 17 mS/cm at about 20° C., about 17.5 mS/cm at about 20° C., about 18 mS/cm at about 20° C., about 18.5 mS/cm at about 20° C., about 19 mS/cm at about 20° C., about 19.5 mS/cm at about 20° C., or about 20 mS/cm at about 20° C. In certain embodiments, the pharmaceutical composition has a conductivity of about 17 mS/cm at about 20° C.

The pharmaceutical composition of the present disclosure may have an osmolality of about 350 mOsm/kg to about 550 mOsm/kg, for example, about 350 mOsm/kg to about 400 mOsm/kg, about 400 mOsm/kg to about 450 mOsm/kg, about 450 mOsm/kg to about 500 mOsm/kg, or about 500 mOsm/kg to about 550 mOsm/kg. In certain embodiments, the pharmaceutical composition has an osmolality of about 350 mOsm/kg, about 360 mOsm/kg, about 370 mOsm/kg, about 380 mOsm/kg, about 390 mOsm/kg, about 400 mOsm/kg, about 410 mOsm/kg, about 420 mOsm/kg, about 430 mOsm/kg, about 440 mOsm/kg, about 450 mOsm/kg, about 460 mOsm/kg, about 470 mOsm/kg, about 480 mOsm/kg, about 490 mOsm/kg, or about 500 mOsm/kg. In certain embodiments, the pharmaceutical composition has an osmolality of about 450 mOsm/kg.

### IV. Adeno-Associated Virus Formulations

The present disclosure provides pharmaceutical compositions comprising an adeno-associated virus (AAV) and one or more excipients in an amount that stabilizes the AAV, thus allowing for maintained viral titers, maintained AAV vector purity, improved control over the level of aggregates (e.g., reduced accumulation of aggregates), reduced AAV degradation products, and prevents non-specific binding of the AAV to surfaces. The pharmaceutical compositions described herein are suitable for long-term storage of AAV (e.g., rAAV) and are able to resist thermal stress (e.g., incubation at high temperatures such as 25° C. and 40° C.).

In certain embodiments, a pharmaceutical composition of the present disclosure comprises an AAV (e.g., rAAV), a buffering agent, a salt, and a stabilizing agent. In certain embodiments, the pharmaceutical composition comprises an rAAV, a buffering agent, a salt, and a stabilizing agent.

In certain embodiments, the pharmaceutical composition comprises an AAV (e.g., rAAV), histidine, a salt, and a stabilizing agent. In certain embodiments, the pharmaceutical composition comprises an AAV (e.g., rAAV), about 5 mM to about 50 mM histidine, a salt, and a stabilizing agent. In certain embodiments, the pharmaceutical composition comprises an AAV (e.g., rAAV), about 20 mM histidine, a salt, and a stabilizing agent. In certain embodiments, the pharmaceutical composition comprises an AAV (e.g., rAAV), sodium phosphate, a salt, and a stabilizing agent. In certain embodiments, the pharmaceutical composition comprises an AAV (e.g., rAAV), about 5 mM to about 50 mM sodium phosphate, a salt, and a stabilizing agent. In certain embodiments, the pharmaceutical composition comprises an AAV (e.g., rAAV), about 20 mM sodium phosphate, a salt, and a stabilizing agent. In certain embodiments, the pharmaceutical composition has a pH of about 6 to about 8. In certain embodiments, the pharmaceutical composition has a pH of about 6.3 to about 8.3. In certain embodiments, the pharmaceutical composition has a pH of about 7.3.

In certain embodiments, the pharmaceutical composition comprises an AAV (e.g., rAAV), a buffering agent, sodium chloride, and a stabilizing agent. In certain embodiments, the pharmaceutical composition comprises an AAV (e.g., rAAV), a buffering agent, greater than about 150 mM sodium chloride, and a stabilizing agent. In certain embodiments, the pharmaceutical composition comprises an AAV (e.g., rAAV), a buffering agent, about 150 mM to about 200 mM sodium chloride, and a stabilizing agent. In certain embodiments, the pharmaceutical composition comprises an AAV (e.g., rAAV), a buffering agent, about 175 mM sodium chloride, and a stabilizing agent. In certain embodiments, the pharmaceutical composition has a pH of about 6 to about 8. In certain embodiments, the pharmaceutical composition has a pH of about 6.3 to about 8.3. In certain embodiments, the pharmaceutical composition has a pH of about 7.3.

In certain embodiments, the pharmaceutical composition comprises an AAV (e.g., rAAV), a buffering agent, a salt, and trehalose. In certain embodiments, the pharmaceutical composition comprises an AAV (e.g., rAAV), a buffering agent, a salt, and about 1% (w/v) to about 10% (w/v) trehalose. In certain embodiments, the pharmaceutical composition comprises an AAV (e.g., rAAV), a buffering agent, a salt, and about 1% (w/v) trehalose. In certain embodiments, the pharmaceutical composition comprises an AAV (e.g., rAAV), a buffering agent, a salt, and about 3% (w/v) trehalose. In certain embodiments, the pharmaceutical composition comprises an AAV (e.g., rAAV), a buffering agent, a salt, and about 5% (w/v) trehalose. In certain embodiments, the pharmaceutical composition has a pH of about 6 to about 8. In certain embodiments, the pharmaceutical composition has a pH of about 6.3 to about 8.3. In certain embodiments, the pharmaceutical composition has a pH of about 7.3.

In certain embodiments, the pharmaceutical composition further comprises a surfactant. In certain embodiments, the pharmaceutical composition comprises an AAV (e.g., rAAV), a buffering agent, a salt, a stabilizing agent, and a surfactant. In certain embodiments, the pharmaceutical composition comprises an AAV (e.g., rAAV), a buffering agent, a salt, a stabilizing agent, and a non-ionic surfactant. In certain embodiments, the pharmaceutical composition comprises an AAV (e.g., rAAV), a buffering agent, a salt, a stabilizing agent, and Poloxamer 188 (P188). In certain embodiments, the pharmaceutical composition comprises an AAV (e.g., rAAV), a buffering agent, a salt, a stabilizing agent, and about 0.01% (w/v) to about 0.05% (w/v) Poloxamer 188 (P188). In certain embodiments, the pharmaceutical composition comprises an AAV (e.g., rAAV), a buffering agent, a salt, a stabilizing agent, and about 0.03% (w/v) Poloxamer 188 (P188). In certain embodiments, the pharmaceutical composition has a pH of about 6 to about 8. In certain embodiments, the pharmaceutical composition has a pH of about 6.3 to about 8.3. In certain embodiments, the pharmaceutical composition has a pH of about 7.3.

In certain embodiments, the pharmaceutical composition comprises at least about 1e13 vg/ml of the AAV (e.g., rAAV). In certain embodiments, the pharmaceutical composition comprises at least about 2e13 vg/ml of the AAV (e.g., rAAV). In certain embodiments, the pharmaceutical composition comprises about 2e13 vg/ml to about 2e14 vg/ml of the AAV (e.g., rAAV). In certain embodiments, the pharmaceutical composition comprises at least about 1e15 vg/ml of the AAV (e.g., rAAV). In certain embodiments, the pharmaceutical composition comprises up to about 6e15 vg/ml of the AAV (e.g., rAAV). In certain embodiments, the pharmaceutical composition comprises about 1e13 vg/ml to about 6e15 vg/ml of the AAV (e.g., rAAV). In certain embodiments, the pharmaceutical composition comprises about 1e13 vg/ml, about 2e13 vg/ml, about 3e13 vg/ml, about 4e13 vg/ml, about 5e13 vg/ml, about 6e13 vg/ml, about 7e13 vg/ml, about 8e13 vg/ml, about 9e13 vg/ml, about 1e14 vg/ml, about 2e14 vg/ml, about 3e14 vg/ml, about 4e14 vg/ml, about 5e14 vg/ml, about 6e14 vg/ml, about 7e14 vg/ml, about 8e14 vg/ml, about 9e14 vg/ml, about 1e15 vg/ml, about 2e15 vg/ml, about 3e15 vg/ml, about 4e15 vg/ml, about 5e15 vg/ml, about 6e15 vg/ml, about 7e15 vg/ml, about 8e15 vg/ml, about 9e15 vg/ml, or about 1e16 vg/ml of the AAV (e.g., rAAV). In certain embodiments, the pharmaceutical composition comprises about 2e13 vg/ml of the AAV (e.g., rAAV). In certain embodiments, the pharmaceutical composition comprises about 6e13 vg/ml of the AAV (e.g., rAAV). In certain embodiments, the pharmaceutical composition comprises about 8e13 vg/ml of the AAV (e.g., rAAV). In certain embodiments, the pharmaceutical composition comprises about 1e14 vg/ml of the AAV (e.g., rAAV). In certain embodiments, the pharmaceutical composition comprises about 2e14 vg/ml of the AAV (e.g., rAAV). In certain embodiments, the pharmaceutical composition comprises about 1e15 vg/ml of the AAV (e.g., rAAV). In certain embodiments, the pharmaceutical composition comprises about 6e15 vg/ml of the AAV (e.g., rAAV).

In certain embodiments, the pharmaceutical composition comprises about 1e13 capsids/ml to about 1e16 capsids/ml of the AAV (e.g., rAAV). In certain embodiments, the pharmaceutical composition comprises about 5e13 capsids/ml to about 8e15 capsids/ml of the AAV (e.g., rAAV). In certain embodiments, the pharmaceutical composition comprises about 1e13 capsids/ml, about 2e13 capsids/ml, about 3e13 capsids/ml, about 4e13 capsids/ml, about 5e13 capsids/ml, about 6e13 capsids/ml, about 7e13 capsids/ml, about 8e13 capsids/ml, about 9e13 capsids/ml, about 1e14 capsids/ml, about 2e14 capsids/ml, about 3e14 capsids/ml, about 4e14 capsids/ml, about 5e14 capsids/ml, about 6e14 capsids/ml, about 7e14 capsids/ml, about 8e14 capsids/ml, about 9e14 capsids/ml, about 1e15 capsids/ml, about 2e15 capsids/ml, about 3e15 capsids/ml, about 4e15 capsids/ml, about 5e15 capsids/ml, about 6e15 capsids/ml, about 7e15 capsids/ml, about 8e15 capsids/ml, about 9e15 capsids/ml, or about 1e16 capsids/ml of the AAV (e.g., rAAV). In certain embodiments, the pharmaceutical composition comprises about 1e14 capsids/ml of the AAV (e.g., rAAV).

In certain embodiments, the pharmaceutical composition comprises an AAV (e.g., rAAV), histidine, sodium chloride, trehalose, and Poloxamer 188 (P188). In certain embodiments, the pharmaceutical composition comprises an AAV (e.g., rAAV), about 5 mM to about 50 mM histidine, about 1% (w/v) to about 10% (w/v) trehalose, greater than about 150 mM sodium chloride, and about 0.01% (w/v) to about 0.05% (w/v) P188. In certain embodiments, the pharmaceutical composition comprises an AAV (e.g., rAAV), about 20 mM histidine, about 1% (w/v) to about 10% (w/v) trehalose, greater than about 150 mM sodium chloride, and about 0.01% (w/v) to about 0.05% (w/v) P188. In certain embodiments, the pharmaceutical composition comprises an AAV (e.g., rAAV), about 5 mM to about 50 mM histidine, about 3% (w/v) trehalose, greater than about 150 mM sodium chloride, and about 0.01% (w/v) to about 0.05% (w/v) P188. In certain embodiments, the pharmaceutical composition comprises an AAV (e.g., rAAV), about 5 mM to about 50 mM histidine, about 1% (w/v) to about 10% (w/v) trehalose, about 175 mM sodium chloride, and about 0.01% (w/v) to about 0.05% (w/v) P188. In certain embodiments, the pharmaceutical composition comprises an AAV (e.g., rAAV), about 5 mM to about 50 mM histidine, about 1% (w/v) to about 10% (w/v) trehalose, greater than about 150 mM sodium chloride, and about 0.03% (w/v) P188. In certain embodiments, the pharmaceutical composition has a pH of about 6 to about 8. In certain embodiments, the pharmaceutical composition has a pH of about 6.3 to about 8.3. In certain embodiments, the pharmaceutical composition has a pH of about 7.3.

In certain embodiments, the pharmaceutical composition comprises an AAV (e.g., rAAV), about 20 mM histidine, about 3% (w/v) trehalose, greater than about 150 mM sodium chloride, and about 0.01% (w/v) to about 0.05% (w/v) P188. In certain embodiments, the pharmaceutical composition comprises an AAV (e.g., rAAV), about 20 mM histidine, about 1% (w/v) to about 10% (w/v) trehalose, about 175 mM sodium chloride, and about 0.01% (w/v) to about 0.05% (w/v) P188. In certain embodiments, the pharmaceutical composition comprises an AAV (e.g., rAAV), about 20 mM histidine, about 1% (w/v) to about 10% (w/v) trehalose, greater than about 150 mM sodium chloride, about 0.03% (w/v) P188. In certain embodiments, the pharmaceutical composition comprises an AAV (e.g., rAAV), about 5 mM to about 50 mM histidine, about 3% (w/v) trehalose, about 175 mM sodium chloride, and about 0.01% (w/v) to about 0.05% (w/v) P188. In certain embodiments, the pharmaceutical composition comprises an AAV (e.g., rAAV), about 5 mM to about 50 mM histidine, about 3% (w/v) trehalose, greater than about 150 mM sodium chloride, and about 0.03% (w/v) P188. In certain embodiments, the pharmaceutical composition comprises an AAV (e.g., rAAV), about 5 mM to about 50 mM histidine, about 1% (w/v) to about 10% (w/v) trehalose, about 175 mM sodium chloride, and about 0.03% (w/v) P188. In certain embodiments, the pharmaceutical composition comprises an AAV (e.g., rAAV), about 20 mM histidine, about 3% (w/v) trehalose, about 175 mM sodium chloride, and about 0.01% (w/v) to about 0.05% (w/v) P188. In certain embodiments, the pharmaceutical composition comprises an AAV (e.g., rAAV), about 5 mM to about 50 mM histidine, about 3% (w/v) trehalose, about 175 mM sodium chloride, and about 0.03% (w/v) P188. In certain embodiments, the pharmaceutical composition comprises an AAV (e.g., rAAV), about 20 mM histidine, about 3% (w/v) trehalose, about 175 mM sodium chloride, and about 0.03% (w/v) P188. In certain embodiments, the pharmaceutical composition has a pH of about 6 to about 8. In certain embodiments, the pharmaceutical composition has a pH of about 6.3 to about 8.3. In certain embodiments, the pharmaceutical composition has a pH of about 7.3.

In certain embodiments, the pharmaceutical composition comprises about 1e13 vg/ml to about 6e15 vg/ml of an AAV (e.g., rAAV), histidine, sodium chloride, trehalose, and Poloxamer 188 (P188). In certain embodiments, the pharmaceutical composition comprises about 1e13 vg/ml to about 6e15 vg/ml of an AAV (e.g., rAAV), about 5 mM to about 50 mM histidine, about 1% (w/v) to about 10% (w/v) trehalose, greater than about 150 mM sodium chloride, and about 0.01% (w/v) to about 0.05% (w/v) P188. In certain embodiments, the pharmaceutical composition comprises about 1e13 vg/ml to about 6e15 vg/ml of an AAV (e.g., rAAV), about 20 mM histidine, about 1% (w/v) to about 10% (w/v) trehalose, greater than about 150 mM sodium chloride, and about 0.01% (w/v) to about 0.05% (w/v) P188. In certain embodiments, the pharmaceutical composition comprises about 1e13 vg/ml to about 6e15 vg/ml of an AAV (e.g., rAAV), about 5 mM to about 50 mM histidine, about 3% (w/v) trehalose, greater than about 150 mM sodium chloride, and about 0.01% (w/v) to about 0.05% (w/v) P188. In certain embodiments, the pharmaceutical composition comprises about 1e13 vg/ml to about 6e15 vg/ml of an AAV (e.g., rAAV), about 5 mM to about 50 mM histidine, about 1% (w/v) to about 10% (w/v) trehalose, about 175 mM sodium chloride, and about 0.01% (w/v) to about 0.05% (w/v) P188. In certain embodiments, the pharmaceutical composition comprises about 1e13 vg/ml to about 6e15 vg/ml of an AAV (e.g., rAAV), about 5 mM to about 50 mM histidine, about 1% (w/v) to about 10% (w/v) trehalose, greater than about 150 mM sodium chloride, and about 0.03% (w/v) P188. In certain embodiments, the pharmaceutical composition comprises about 1e13 vg/ml to about 1e15 vg/ml of an AAV (e.g., rAAV), histidine, sodium chloride, trehalose, and Poloxamer 188 (P188). In certain embodiments, the pharmaceutical composition comprises about 1e13 vg/ml to about 1e15 vg/ml of an AAV (e.g., rAAV), about 5 mM to about 50 mM histidine, about 1% (w/v) to about 10% (w/v) trehalose, greater than about 150 mM sodium chloride, and about 0.01% (w/v) to about 0.05% (w/v) P188. In certain embodiments, the pharmaceutical composition comprises about 1e13 vg/ml to about 1e15 vg/ml of an AAV (e.g., rAAV), about 20 mM histidine, about 1% (w/v) to about 10% (w/v) trehalose, greater than about 150 mM sodium chloride, and about 0.01% (w/v) to about 0.05% (w/v) P188. In certain embodiments, the pharmaceutical composition comprises about 1e13 vg/ml to about 1e15 vg/ml of an AAV (e.g., rAAV), about 5 mM to about 50 mM histidine, about 3% (w/v) trehalose, greater than about 150 mM sodium chloride, and about 0.01% (w/v) to about 0.05% (w/v) P188. In certain embodiments, the pharmaceutical composition comprises about 1e13 vg/ml to about 1e15 vg/ml of an AAV (e.g., rAAV), about 5 mM to about 50 mM histidine, about 1% (w/v) to about 10% (w/v) trehalose, about 175 mM sodium chloride, and about 0.01% (w/v) to about 0.05% (w/v) P188. In certain embodiments, the pharmaceutical composition comprises about 1e13 vg/ml to about 1e15 vg/ml of an AAV (e.g., rAAV), about 5 mM to about 50 mM histidine, about 1% (w/v) to about 10% (w/v) trehalose, greater than about 150 mM sodium chloride, and about 0.03% (w/v) P188. In certain embodiments, the pharmaceutical composition has a pH of about 6 to about 8. In certain embodiments, the pharmaceutical composition has a pH of about 6.3 to about 8.3. In certain embodiments, the pharmaceutical composition has a pH of about 7.3.

In certain embodiments, the pharmaceutical composition comprises about 1e13 vg/ml to about 6e15 vg/ml of an AAV (e.g., rAAV), about 20 mM histidine, about 3% (w/v) trehalose, greater than about 150 mM sodium chloride, and about 0.01% (w/v) to about 0.05% (w/v) P188. In certain embodiments, the pharmaceutical composition comprises about 1e13 vg/ml to about 6e15 vg/ml of an AAV (e.g., rAAV), about 20 mM histidine, about 1% (w/v) to about 10% (w/v) trehalose, about 175 mM sodium chloride, and about 0.01% (w/v) to about 0.05% (w/v) P188. In certain embodiments, the pharmaceutical composition comprises about 1e13 vg/ml to about 6e15 vg/ml of an AAV (e.g., rAAV), about 20 mM histidine, about 1% (w/v) to about 10% (w/v) trehalose, greater than about 150 mM sodium chloride, about 0.03% (w/v) P188. In certain embodiments, the pharmaceutical composition comprises about 1e13 vg/ml to about 6e15 vg/ml of an AAV (e.g., rAAV), about 5 mM to about 50 mM histidine, about 3% (w/v) trehalose, about 175 mM sodium chloride, and about 0.01% (w/v) to about 0.05% (w/v) P188. In certain embodiments, the pharmaceutical composition comprises about 1e13 vg/ml to about 6e15 vg/ml of an AAV (e.g., rAAV), about 5 mM to about 50 mM histidine, about 3% (w/v) trehalose, greater than about 150 mM sodium chloride, and about 0.03% (w/v) P188. In certain embodiments, the pharmaceutical composition comprises about 1e13 vg/ml to about 6e15 vg/ml of an AAV (e.g., rAAV), about 5 mM to about 50 mM histidine, about 1% (w/v) to about 10% (w/v) trehalose, about 175 mM sodium chloride, and about 0.03% (w/v) P188. In certain embodiments, the pharmaceutical composition comprises about 1e13 vg/ml to about 6e15 vg/ml of an AAV (e.g., rAAV), about 20 mM histidine, about 3% (w/v) trehalose, about 175 mM sodium chloride, and about 0.01% (w/v) to about 0.05% (w/v) P188. In certain embodiments, the pharmaceutical composition comprises about 1e13 vg/ml to about 6e15 vg/ml of an AAV (e.g., rAAV), about 5 mM to about 50 mM histidine, about 3% (w/v) trehalose, about 175 mM sodium chloride, and about 0.03% (w/v) P188. In certain embodiments, the pharmaceutical composition comprises about 1e13 vg/ml to about 6e15 vg/ml of an AAV (e.g., rAAV), about 20 mM histidine, about 3% (w/v) trehalose, about 175 mM sodium chloride, and about 0.03% (w/v) P188. In certain embodiments, the pharmaceutical composition comprises about 1e13 vg/ml to about 1e15 vg/ml of an AAV (e.g., rAAV), about 20 mM histidine, about 3% (w/v) trehalose, greater than about 150 mM sodium chloride, and about 0.01% (w/v) to about 0.05% (w/v) P188. In certain embodiments, the pharmaceutical composition comprises about 1e13 vg/ml to about 1e15 vg/ml of an AAV (e.g., rAAV), about 20 mM histidine, about 1% (w/v) to about 10% (w/v) trehalose, about 175 mM sodium chloride, and about 0.01% (w/v) to about 0.05% (w/v) P188. In certain embodiments, the pharmaceutical composition comprises about 1e13 vg/ml to about 1e15 vg/ml of an AAV (e.g., rAAV), about 20 mM histidine, about 1% (w/v) to about 10% (w/v) trehalose, greater than about 150 mM sodium chloride, about 0.03% (w/v) P188. In certain embodiments, the pharmaceutical composition comprises about 1e13 vg/ml to about 1e15 vg/ml of an AAV (e.g., rAAV), about 5 mM to about 50 mM histidine, about 3% (w/v) trehalose, about 175 mM sodium chloride, and about 0.01% (w/v) to about 0.05% (w/v) P188. In certain embodiments, the pharmaceutical composition comprises about 1e13 vg/ml to about 1e15 vg/ml of an AAV (e.g., rAAV), about 5 mM to about 50 mM histidine, about 3% (w/v) trehalose, greater than about 150 mM sodium chloride, and about 0.03% (w/v) P188. In certain embodiments, the pharmaceutical composition comprises about 1e13 vg/ml to about 1e15 vg/ml of an AAV (e.g., rAAV), about 5 mM to about 50 mM histidine, about 1% (w/v) to about 10% (w/v) trehalose, about 175 mM sodium chloride, and about 0.03% (w/v) P188. In certain embodiments, the pharmaceutical composition comprises about 1e13 vg/ml to about 1e15 vg/ml of an AAV (e.g., rAAV), about 20 mM histidine, about 3% (w/v) trehalose, about 175 mM sodium chloride, and about 0.01% (w/v) to about 0.05% (w/v) P188. In certain embodiments, the pharmaceutical composition comprises about 1e13 vg/ml to about 1e15 vg/ml of an AAV (e.g., rAAV), about 5 mM to about 50 mM histidine, about 3% (w/v) trehalose, about 175 mM sodium chloride, and about 0.03% (w/v) P188. In certain embodiments, the pharmaceutical composition comprises about 1e13 vg/ml to about 1e15 vg/ml of an AAV (e.g., rAAV), about 20 mM histidine, about 3% (w/v) trehalose, about 175 mM sodium chloride, and about 0.03% (w/v) P188. In certain embodiments, the pharmaceutical composition has a pH of about 6 to about 8. In certain embodiments, the pharmaceutical composition has a pH of about 6.3 to about 8.3. In certain embodiments, the pharmaceutical composition has a pH of about 7.3.

In certain embodiments, the pharmaceutical composition comprises about 1e13 vg/ml to about 6e15 vg/ml of an AAV (e.g., rAAV), histidine, sodium chloride, trehalose, and Poloxamer 188 (P188), wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition comprises about 1e13 vg/ml to about 6e15 vg/ml of an AAV (e.g., rAAV), about 5 mM to about 50 mM histidine, about 1% (w/v) to about 10% (w/v) trehalose, greater than about 150 mM sodium chloride, and about 0.01% (w/v) to about 0.05% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition comprises about 1e13 vg/ml to about 6e15 vg/ml of an AAV (e.g., rAAV), about 20 mM histidine, about 1% (w/v) to about 10% (w/v) trehalose, greater than about 150 mM sodium chloride, and about 0.01% (w/v) to about 0.05% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition comprises about 1e13 vg/ml to about 6e15 vg/ml of an AAV (e.g., rAAV), about 5 mM to about 50 mM histidine, about 3% (w/v) trehalose, greater than about 150 mM sodium chloride, and about 0.01% (w/v) to about 0.05% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition comprises about 1e13 vg/ml to about 6e15 vg/ml of an AAV (e.g., rAAV), about 5 mM to about 50 mM histidine, about 1% (w/v) to about 10% (w/v) trehalose, about 175 mM sodium chloride, and about 0.01% (w/v) to about 0.05% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition comprises about 1e13 vg/ml to about 6e15 vg/ml of an AAV (e.g., rAAV), about 5 mM to about 50 mM histidine, about 1% (w/v) to about 10% (w/v) trehalose, greater than about 150 mM sodium chloride, and about 0.03% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition has a pH of about 6 to about 8. In certain embodiments, the pharmaceutical composition has a pH of about 6.3 to about 8.3. In certain embodiments, the pharmaceutical composition has a pH of about 7.3.

In certain embodiments, the pharmaceutical composition comprises about 1e13 vg/ml to about 6e15 vg/ml of an AAV (e.g., rAAV), about 20 mM histidine, about 3% (w/v) trehalose, greater than about 150 mM sodium chloride, and about 0.01% (w/v) to about 0.05% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition comprises about 1e13 vg/ml to about 6e15 vg/ml of an AAV (e.g., rAAV), about 20 mM histidine, about 1% (w/v) to about 10% (w/v) trehalose, about 175 mM sodium chloride, and about 0.01% (w/v) to about 0.05% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition comprises about 1e13 vg/ml to about 6e15 vg/ml of an AAV (e.g., rAAV), about 20 mM histidine, about 1% (w/v) to about 10% (w/v) trehalose, greater than about 150 mM sodium chloride, about 0.03% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition comprises about 1e13 vg/ml to about 6e15 vg/ml of an AAV (e.g., rAAV), about 5 mM to about 50 mM histidine, about 3% (w/v) trehalose, about 175 mM sodium chloride, and about 0.01% (w/v) to about 0.05% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition comprises about 1e13 vg/ml to about 6e15 vg/ml of an AAV (e.g., rAAV), about 5 mM to about 50 mM histidine, about 3% (w/v) trehalose, greater than about 150 mM sodium chloride, and about 0.03% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition comprises about 1e13 vg/ml to about 6e15 vg/ml of an AAV (e.g., rAAV), about 5 mM to about 50 mM histidine, about 1% (w/v) to about 10% (w/v) trehalose, about 175 mM sodium chloride, and about 0.03% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition comprises about 1e13 vg/ml to about 6e15 vg/ml of an AAV (e.g., rAAV), about 20 mM histidine, about 3% (w/v) trehalose, about 175 mM sodium chloride, and about 0.01% (w/v) to about 0.05% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition comprises about 1e13 vg/ml to about 6e15 vg/ml of an AAV (e.g., rAAV), about 5 mM to about 50 mM histidine, about 3% (w/v) trehalose, about 175 mM sodium chloride, and about 0.03% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition comprises about 1e13 vg/ml to about 6e15 vg/ml of an AAV (e.g., rAAV), about 20 mM histidine, about 3% (w/v) trehalose, about 175 mM sodium chloride, and about 0.03% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition has a pH of about 6 to about 8. In certain embodiments, the pharmaceutical composition has a pH of about 6.3 to about 8.3. In certain embodiments, the pharmaceutical composition has a pH of about 7.3.

In certain embodiments, the pharmaceutical composition comprises about 1e13 vg/ml to about 6e15 vg/ml of an rAAV comprising the nucleotide sequence set forth in SEQ ID NO: 54 or 56, about 20 mM histidine, about 3% (w/v) trehalose, about 175 mM sodium chloride, and about 0.03% (w/v) P188, wherein the pharmaceutical composition has a pH of about 7.3. In certain embodiments, the pharmaceutical composition comprises about 2e13 vg/ml of an rAAV comprising the nucleotide sequence set forth in SEQ ID NO: 54 or 56, about 20 mM histidine, about 3% (w/v) trehalose, about 175 mM sodium chloride, and about 0.03% (w/v) P188, wherein the pharmaceutical composition has a pH of about 7.3. In certain embodiments, the pharmaceutical composition comprises about 6e13 vg/ml of an rAAV comprising the nucleotide sequence set forth in SEQ ID NO: 54 or 56, about 20 mM histidine, about 3% (w/v) trehalose, about 175 mM sodium chloride, and about 0.03% (w/v) P188, wherein the pharmaceutical composition has a pH of about 7.3. In certain embodiments, the pharmaceutical composition comprises about 8e13 vg/ml of an rAAV comprising the nucleotide sequence set forth in SEQ ID NO: 54 or 56, about 20 mM histidine, about 3% (w/v) trehalose, about 175 mM sodium chloride, and about 0.03% (w/v) P188, wherein the pharmaceutical composition has a pH of about 7.3. In certain embodiments, the pharmaceutical composition comprises about 1e14 vg/ml of an rAAV comprising the nucleotide sequence set forth in SEQ ID NO: 54 or 56, about 20 mM histidine, about 3% (w/v) trehalose, about 175 mM sodium chloride, and about 0.03% (w/v) P188, wherein the pharmaceutical composition has a pH of about 7.3. In certain embodiments, the pharmaceutical composition comprises about 2e14 vg/ml of an rAAV comprising the nucleotide sequence set forth in SEQ ID NO: 54 or 56, about 20 mM histidine, about 3% (w/v) trehalose, about 175 mM sodium chloride, and about 0.03% (w/v) P188, wherein the pharmaceutical composition has a pH of about 7.3.

In certain embodiments, the pharmaceutical composition comprises an AAV (e.g., rAAV), sodium phosphate, sodium chloride, sucrose, and Poloxamer 188 (P188). In certain embodiments, the pharmaceutical composition comprises an AAV (e.g., rAAV), about 5 mM to about 50 mM sodium phosphate, about 1% (w/v) to about 10% (w/v) sucrose, greater than about 150 mM sodium chloride, and about 0.01% (w/v) to about 0.05% (w/v) P188. In certain embodiments, the pharmaceutical composition comprises an AAV (e.g., rAAV), about 20 mM sodium phosphate, about 1% (w/v) to about 10% (w/v) sucrose, greater than about 150 mM sodium chloride, and about 0.01% (w/v) to about 0.05% (w/v) P188. In certain embodiments, the pharmaceutical composition comprises an AAV (e.g., rAAV), about 5 mM to about 50 mM sodium phosphate, about 1% (w/v) sucrose, greater than about 150 mM sodium chloride, and about 0.01% (w/v) to about 0.05% (w/v) P188. In certain embodiments, the pharmaceutical composition comprises an AAV (e.g., rAAV), about 5 mM to about 50 mM sodium phosphate, about 1% (w/v) to about 10% (w/v) sucrose, about 175 mM sodium chloride, and about 0.01% (w/v) to about 0.05% (w/v) P188. In certain embodiments, the pharmaceutical composition comprises an AAV (e.g., rAAV), about 5 mM to about 50 mM sodium phosphate, about 1% (w/v) to about 10% (w/v) sucrose, greater than about 150 mM sodium chloride, and about 0.03% (w/v) P188. In certain embodiments, the pharmaceutical composition has a pH of about 6 to about 8. In certain embodiments, the pharmaceutical composition has a pH of about 6.3 to about 8.3. In certain embodiments, the pharmaceutical composition has a pH of about 7.3.

In certain embodiments, the pharmaceutical composition comprises an AAV (e.g., rAAV), about 20 mM sodium phosphate, about 1% (w/v) sucrose, greater than about 150 mM sodium chloride, and about 0.01% (w/v) to about 0.05% (w/v) P188. In certain embodiments, the pharmaceutical composition comprises an AAV (e.g., rAAV), about 20 mM sodium phosphate, about 1% (w/v) to about 10% (w/v) sucrose, about 175 mM sodium chloride, and about 0.01% (w/v) to about 0.05% (w/v) P188. In certain embodiments, the pharmaceutical composition comprises an AAV (e.g., rAAV), about 20 mM sodium phosphate, about 1% (w/v) to about 10% (w/v) trehalose, greater than about 150 mM sodium chloride, about 0.03% (w/v) P188. In certain embodiments, the pharmaceutical composition comprises an AAV (e.g., rAAV), about 5 mM to about 50 mM sodium phosphate, about 1% (w/v) sucrose, about 175 mM sodium chloride, and about 0.01% (w/v) to about 0.05% (w/v) P188. In certain embodiments, the pharmaceutical composition comprises an AAV (e.g., rAAV), about 5 mM to about 50 mM sodium phosphate, about 1% (w/v) sucrose, greater than about 150 mM sodium chloride, and about 0.03% (w/v) P188. In certain embodiments, the pharmaceutical composition comprises an AAV (e.g., rAAV), about 5 mM to about 50 mM sodium phosphate, about 1% (w/v) to about 10% (w/v) sucrose, about 175 mM sodium chloride, and about 0.03% (w/v) P188. In certain embodiments, the pharmaceutical composition comprises an AAV (e.g., rAAV), about 20 mM sodium phosphate, about 1% (w/v) sucrose, about 175 mM sodium chloride, and about 0.01% (w/v) to about 0.05% (w/v) P188. In certain embodiments, the pharmaceutical composition comprises an AAV (e.g., rAAV), about 5 mM to about 50 mM sodium phosphate, about 1% (w/v) sucrose, about 175 mM sodium chloride, and about 0.03% (w/v) P188. In certain embodiments, the pharmaceutical composition comprises an AAV (e.g., rAAV), about 20 mM sodium phosphate, about 1% (w/v) sucrose, about 175 mM sodium chloride, and about 0.03% (w/v) P188. In certain embodiments, the pharmaceutical composition has a pH of about 6 to about 8. In certain embodiments, the pharmaceutical composition has a pH of about 6.3 to about 8.3. In certain embodiments, the pharmaceutical composition has a pH of about 7.3.

In certain embodiments, the pharmaceutical composition comprises about 1e13 vg/ml to about 6e15 vg/ml of an AAV (e.g., rAAV), sodium phosphate, sodium chloride, sucrose, and Poloxamer 188 (P188). In certain embodiments, the pharmaceutical composition comprises about 1e13 vg/ml to about 6e15 vg/ml of an AAV (e.g., rAAV), about 5 mM to about 50 mM sodium phosphate, about 1% (w/v) to about 10% (w/v) sucrose, greater than about 150 mM sodium chloride, and about 0.01% (w/v) to about 0.05% (w/v) P188. In certain embodiments, the pharmaceutical composition comprises about 1e13 vg/ml to about 6e15 vg/ml of an AAV (e.g., rAAV), about 20 mM sodium phosphate, about 1% (w/v) to about 10% (w/v) sucrose, greater than about 150 mM sodium chloride, and about 0.01% (w/v) to about 0.05% (w/v) P188. In certain embodiments, the pharmaceutical composition comprises about 1e13 vg/ml to about 6e15 vg/ml of an AAV (e.g., rAAV), about 5 mM to about 50 mM sodium phosphate, about 1% (w/v) sucrose, greater than about 150 mM sodium chloride, and about 0.01% (w/v) to about 0.05% (w/v) P188. In certain embodiments, the pharmaceutical composition comprises about 1e13 vg/ml to about 6e15 vg/ml of an AAV (e.g., rAAV), about 5 mM to about 50 mM sodium phosphate, about 1% (w/v) to about 10% (w/v) sucrose, about 175 mM sodium chloride, and about 0.01% (w/v) to about 0.05% (w/v) P188. In certain embodiments, the pharmaceutical composition comprises about 1e13 vg/ml to about 6e15 vg/ml of an AAV (e.g., rAAV), about 5 mM to about 50 mM sodium phosphate, about 1% (w/v) to about 10% (w/v) sucrose, greater than about 150 mM sodium chloride, and about 0.03% (w/v) P188. In certain embodiments, the pharmaceutical composition has a pH of about 6 to about 8. In certain embodiments, the pharmaceutical composition has a pH of about 6.3 to about 8.3. In certain embodiments, the pharmaceutical composition has a pH of about 7.3.

In certain embodiments, the pharmaceutical composition comprises about 1e13 vg/ml to about 6e15 vg/ml of an AAV (e.g., rAAV), about 20 mM sodium phosphate, about 1% (w/v) sucrose, greater than about 150 mM sodium chloride, and about 0.01% (w/v) to about 0.05% (w/v) P188. In certain embodiments, the pharmaceutical composition comprises about 1e13 vg/ml to about 6e15 vg/ml of an AAV (e.g., rAAV), about 20 mM sodium phosphate, about 1% (w/v) to about 10% (w/v) sucrose, about 175 mM sodium chloride, and about 0.01% (w/v) to about 0.05% (w/v) P188. In certain embodiments, the pharmaceutical composition comprises about 1e13 vg/ml to about 6e15 vg/ml of an AAV (e.g., rAAV), about 20 mM sodium phosphate, about 1% (w/v) to about 10% (w/v) trehalose, greater than about 150 mM sodium chloride, about 0.03% (w/v) P188. In certain embodiments, the pharmaceutical composition comprises about 1e13 vg/ml to about 6e15 vg/ml of an AAV (e.g., rAAV), about 5 mM to about 50 mM sodium phosphate, about 1% (w/v) sucrose, about 175 mM sodium chloride, and about 0.01% (w/v) to about 0.05% (w/v) P188. In certain embodiments, the pharmaceutical composition comprises about 1e13 vg/ml to about 6e15 vg/ml of an AAV (e.g., rAAV), about 5 mM to about 50 mM sodium phosphate, about 1% (w/v) sucrose, greater than about 150 mM sodium chloride, and about 0.03% (w/v) P188. In certain embodiments, the pharmaceutical composition comprises about 1e13 vg/ml to about 6e15 vg/ml of an AAV (e.g., rAAV), about 5 mM to about 50 mM sodium phosphate, about 1% (w/v) to about 10% (w/v) sucrose, about 175 mM sodium chloride, and about 0.03% (w/v) P188. In certain embodiments, the pharmaceutical composition comprises about 1e13 vg/ml to about 6e15 vg/ml of an AAV (e.g., rAAV), about 20 mM sodium phosphate, about 1% (w/v) sucrose, about 175 mM sodium chloride, and about 0.01% (w/v) to about 0.05% (w/v) P188. In certain embodiments, the pharmaceutical composition comprises about 1e13 vg/ml to about 6e15 vg/ml of an AAV (e.g., rAAV), about 5 mM to about 50 mM sodium phosphate, about 1% (w/v) sucrose, about 175 mM sodium chloride, and about 0.03% (w/v) P188. In certain embodiments, the pharmaceutical composition comprises about 1e13 vg/ml to about 6e15 vg/ml of an AAV (e.g., rAAV), about 20 mM sodium phosphate, about 1% (w/v) sucrose, about 175 mM sodium chloride, and about 0.03% (w/v) P188. In certain embodiments, the pharmaceutical composition has a pH of about 6 to about 8. In certain embodiments, the pharmaceutical composition has a pH of about 6.3 to about 8.3. In certain embodiments, the pharmaceutical composition has a pH of about 7.3.

In certain embodiments, the pharmaceutical composition comprises about 1e13 vg/ml to about 6e15 vg/ml of an AAV (e.g., rAAV), sodium phosphate, sodium chloride, sucrose, and Poloxamer 188 (P188), wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition comprises about 1e13 vg/ml to about 6e15 vg/ml of an AAV (e.g., rAAV), about 5 mM to about 50 mM sodium phosphate, about 1% (w/v) to about 10% (w/v) sucrose, greater than about 150 mM sodium chloride, and about 0.01% (w/v) to about 0.05% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition comprises about 1e13 vg/ml to about 6e15 vg/ml of an AAV (e.g., rAAV), about 20 mM sodium phosphate, about 1% (w/v) to about 10% (w/v) sucrose, greater than about 150 mM sodium chloride, and about 0.01% (w/v) to about 0.05% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition comprises about 1e13 vg/ml to about 6e15 vg/ml of an AAV (e.g., rAAV), about 5 mM to about 50 mM sodium phosphate, about 1% (w/v) sucrose, greater than about 150 mM sodium chloride, and about 0.01% (w/v) to about 0.05% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition comprises about 1e13 vg/ml to about 6e15 vg/ml of an AAV (e.g., rAAV), about 5 mM to about 50 mM sodium phosphate, about 1% (w/v) to about 10% (w/v) sucrose, about 175 mM sodium chloride, and about 0.01% (w/v) to about 0.05% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition comprises about 1e13 vg/ml to about 6e15 vg/ml of an AAV (e.g., rAAV), about 5 mM to about 50 mM sodium phosphate, about 1% (w/v) to about 10% (w/v) sucrose, greater than about 150 mM sodium chloride, and about 0.03% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition has a pH of about 6 to about 8. In certain embodiments, the pharmaceutical composition has a pH of about 6.3 to about 8.3. In certain embodiments, the pharmaceutical composition has a pH of about 7.3.

In certain embodiments, the pharmaceutical composition comprises about 1e13 vg/ml to about 6e15 vg/ml of an AAV (e.g., rAAV), about 20 mM sodium phosphate, about 1% (w/v) sucrose, greater than about 150 mM sodium chloride, and about 0.01% (w/v) to about 0.05% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition comprises about 1e13 vg/ml to about 6e15 vg/ml of an AAV (e.g., rAAV), about 20 mM sodium phosphate, about 1% (w/v) to about 10% (w/v) sucrose, about 175 mM sodium chloride, and about 0.01% (w/v) to about 0.05% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition comprises about 1e13 vg/ml to about 6e15 vg/ml of an AAV (e.g., rAAV), about 20 mM sodium phosphate, about 1% (w/v) to about 10% (w/v) trehalose, greater than about 150 mM sodium chloride, about 0.03% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition comprises about 1e13 vg/ml to about 6e15 vg/ml of an AAV (e.g., rAAV), about 5 mM to about 50 mM sodium phosphate, about 1% (w/v) sucrose, about 175 mM sodium chloride, and about 0.01% (w/v) to about 0.05% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition comprises about 1e13 vg/ml to about 6e15 vg/ml of an AAV (e.g., rAAV), about 5 mM to about 50 mM sodium phosphate, about 1% (w/v) sucrose, greater than about 150 mM sodium chloride, and about 0.03% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition comprises about 1e13 vg/ml to about 6e15 vg/ml of an AAV (e.g., rAAV), about 5 mM to about 50 mM sodium phosphate, about 1% (w/v) to about 10% (w/v) sucrose, about 175 mM sodium chloride, and about 0.03% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition comprises about 1e13 vg/ml to about 6e15 vg/ml of an AAV (e.g., rAAV), about 20 mM sodium phosphate, about 1% (w/v) sucrose, about 175 mM sodium chloride, and about 0.01% (w/v) to about 0.05% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition comprises about 1e13 vg/ml to about 6e15 vg/ml of an AAV (e.g., rAAV), about 5 mM to about 50 mM sodium phosphate, about 1% (w/v) sucrose, about 175 mM sodium chloride, and about 0.03% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition comprises about 1e13 vg/ml to about 6e15 vg/ml of an AAV (e.g., rAAV), about 20 mM sodium phosphate, about 1% (w/v) sucrose, about 175 mM sodium chloride, and about 0.03% (w/v) P188, wherein the AAV is an rAAV comprising an rAAV genome comprising a nucleotide sequence at least 80%, 81%, 82%, 83%, 84%, 85%, 86%, 87%, 88%, 89%, 90%, 91%, 92%, 93%, 94%, 95%, 96%, 97%, 98%, 99%, or 100% identical to the sequence set forth in SEQ ID NO: 50, 51, 52, 53, 54, 55, 56, 57, 58, or 59. In certain embodiments, the pharmaceutical composition has a pH of about 6 to about 8. In certain embodiments, the pharmaceutical composition has a pH of about 6.3 to about 8.3. In certain embodiments, the pharmaceutical composition has a pH of about 7.3.

In certain embodiments, the pharmaceutical composition comprises about 1e13 vg/ml to about 6e15 vg/ml of an rAAV comprising the nucleotide sequence set forth in SEQ ID NO: 54 or 56, about 20 mM sodium phosphate, about 1% (w/v) sucrose, about 175 mM sodium chloride, and about 0.03% (w/v) P188, wherein the pharmaceutical composition has a pH of about 7.3. In certain embodiments, the pharmaceutical composition comprises about 2e13 vg/ml of an rAAV comprising the nucleotide sequence set forth in SEQ ID NO: 54 or 56, about 20 mM sodium phosphate, about 1% (w/v) sucrose, about 175 mM sodium chloride, and about 0.03% (w/v) P188, wherein the pharmaceutical composition has a pH of about 7.3. In certain embodiments, the pharmaceutical composition comprises about 6e13 vg/ml of an rAAV comprising the nucleotide sequence set forth in SEQ ID NO: 54 or 56, about 20 mM sodium phosphate, about 1% (w/v) sucrose, about 175 mM sodium chloride, and about 0.03% (w/v) P188, wherein the pharmaceutical composition has a pH of about 7.3. In certain embodiments, the pharmaceutical composition comprises about 8e13 vg/ml of an rAAV comprising the nucleotide sequence set forth in SEQ ID NO: 54 or 56, about 20 mM sodium phosphate, about 1% (w/v) sucrose, about 175 mM sodium chloride, and about 0.03% (w/v) P188, wherein the pharmaceutical composition has a pH of about 7.3. In certain embodiments, the pharmaceutical composition comprises about 1e14 vg/ml of an rAAV comprising the nucleotide sequence set forth in SEQ ID NO: 54 or 56, about 20 mM sodium phosphate, about 1% (w/v) sucrose, about 175 mM sodium chloride, and about 0.03% (w/v) P188, wherein the pharmaceutical composition has a pH of about 7.3. In certain embodiments, the pharmaceutical composition comprises about 2e14 vg/ml of an rAAV comprising the nucleotide sequence set forth in SEQ ID NO: 54 or 56, about 20 mM sodium phosphate, about 1% (w/v) sucrose, about 175 mM sodium chloride, and about 0.03% (w/v) P188, wherein the pharmaceutical composition has a pH of about 7.3.

### V. Characteristics of the Formulations

Various methods of determining the titer of AAV in a pharmaceutical composition are known, for example, AAV vector genomes can be quantified using droplet digital PCR (ddPCR), and intact AAV particles can be quantified using an enzyme-linked immunosorbent assay utilizing an antibody specific for a conformational epitope on assembled AAV capsids (capsid ELISA). While ddPCR and capsid ELISA provide valuable information regarding the number of AAV vector genomes and intact AAV particles in a composition, other methods are known in the art which can distinguish between intact AAV particles containing vector genomes (“full” capsids) and intact AAV particles that contain partial vector genomes or that do not contain vector genomes (“empty” capsids). Such methods include vector capsid characterization by analytical ultracentrifugation (AUC) that provides a sedimentation profile that can determine the amount of full and empty capsids in a composition.

Various methods of AAV characterization and stability testing are known in the art, including, for example, vector aggregation characterization by size exclusion chromatography (SEC), vector protein characterization by capillary electrophoresis sodium dodecyl sulfate (CE-SDS) analysis, quantitation of residual reagents and plasmid supercoiling such as by reverse-phase chromatography and high performance liquid chromatography (HPLC), and detection and characterization of host cell protein contaminants by liquid chromatography and mass spectrometry (LC-MS).

In certain embodiments, the pharmaceutical compositions of the present disclosure allow for the formulation of high AAV (e.g., rAAV) titers. For example, the pharmaceutical compositions described herein allow for the formulation of greater than about 1e15 vg/ml of AAV (e.g., rAAV). In certain embodiments, the AAV (e.g., rAAV) titer is informed by the vector genome titer which can be determined by ddPCR. In certain embodiments, the pharmaceutical compositions of the present disclosure allow for the maintaining of the vector genome titer after a defined amount of time at a defined temperature. For example, the vector genome titer within the pharmaceutical compositions described herein is maintained after a defined amount of time at a defined temperature, as compared to the vector genome titer determined when the AAV was formulated (e.g., at time 0). In certain embodiments, the vector genome titer within the pharmaceutical compositions described herein after a defined amount of time at a defined temperature is at least about 60%, e.g., about 60%, about 61%, about 62%, about 63%, about 64%, about 65%, about 66%, about 67%, about 68%, about 69%, about 70%, about 71%, about 72%, about 73%, about 74%, about 75%, about 76% about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97% about 98%, about 99%, about 100% of the vector genome titer determined when the AAV was formulated (e.g., at time 0). The defined amount of time can be, e.g., at least about 7 days, at least about 14 days, at least about 21 days, at least about 28 days, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 15 months, at least about 18 months, at least about 24 months, or more after the AAV was formulated. The defined temperature (e.g., the defined temperature at which the pharmaceutical composition is stored at) can be, e.g., about −80° C. to about 45° C., e.g., at about −80° C., about −40° C., about −30° C., about −20° C., about 0° C., about 4°−8° C., about 5° C., about 25° C., about 35° C., about 37° C., or about 45° C. For example, in certain embodiments, the vector genome titer in a pharmaceutical composition as described herein is maintained at least about 60% as compared to the vector genome titer determined when the AAV was formulated, for at least about 12 months after the AAV was formulated when stored at about −80° C. In certain embodiments, the vector genome titer in a pharmaceutical composition as described herein is maintained at least about 60% as compared to the vector genome titer determined when the AAV was formulated, for at least about 24 months after the AAV was formulated when stored at about −80° C. In certain embodiments, the vector genome titer in a pharmaceutical composition as described herein is maintained at least about 60% as compared to the vector genome titer determined when the AAV was formulated, for at least about 12 months after the AAV was formulated when stored at about 5° C. In certain embodiments, the vector genome titer in a pharmaceutical composition as described herein is maintained at least about 60% as compared to the vector genome titer determined when the AAV was formulated, for at least about 24 months after the AAV was formulated when stored at about 5° C. In certain embodiments, the vector genome titer in a pharmaceutical composition as described herein is maintained at least about 60% as compared to the vector genome titer determined when the AAV was formulated, for at least about 6 months after the AAV was formulated when stored at about 25° C. In certain embodiments, the vector genome titer in a pharmaceutical composition as described herein is maintained at least about 60% as compared to the vector genome titer determined when the AAV was formulated, for at least about 2 months after the AAV was formulated when stored at about 40° C.

In certain embodiments, the AAV (e.g., rAAV) titer is informed by the capsid titer which can be determined by capsid ELISA. In certain embodiments, the pharmaceutical compositions of the present disclosure allow for the maintaining of the capsid titer after a defined amount of time at a defined temperature. For example, the capsid titer within the pharmaceutical compositions described herein is maintained after a defined amount of time at a defined temperature, as compared to the capsid titer determined when the AAV was formulated (e.g., at time 0). In certain embodiments, the capsid titer within the pharmaceutical compositions described herein after a defined amount of time at a defined temperature is at least about 60%, e.g., about 60%, about 61%, about 62%, about 63%, about 64%, about 65%, about 66%, about 67%, about 68%, about 69%, about 70%, about 71%, about 72%, about 73%, about 74%, about 75%, about 76% about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97% about 98%, about 99%, about 100% of the capsid titer determined when the AAV was formulated (e.g., at time 0). The defined amount of time can be, e.g., at least about 7 days, at least about 14 days, at least about 21 days, at least about 28 days, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 15 months, at least about 18 months, at least about 24 months, or more after the AAV was formulated. The defined temperature (e.g., the defined temperature at which the pharmaceutical composition is stored at) can be, e.g., about −80° C. to about 45° C., e.g., at about −80° C., about −40° C., about −30° C., about −20° C., about 0° C., about 4°−8° C., about 5° C., about 25° C., about 35° C., about 37° C., or about 45° C. For example, in certain embodiments, the capsid titer in a pharmaceutical composition as described herein is maintained at least about 60% as compared to the capsid titer determined when the AAV was formulated, for at least about 12 months after the AAV was formulated when stored at about −80° C. In certain embodiments, the capsid titer in a pharmaceutical composition as described herein is maintained at least about 60% as compared to the capsid titer determined when the AAV was formulated, for at least about 24 months after the AAV was formulated when stored at about −80° C. In certain embodiments, the capsid titer in a pharmaceutical composition as described herein is maintained at least about 60% as compared to the capsid titer determined when the AAV was formulated, for at least about 12 months after the AAV was formulated when stored at about 5° C. In certain embodiments, the capsid titer in a pharmaceutical composition as described herein is maintained at least about 60% as compared to the capsid titer determined when the AAV was formulated, for at least about 24 months after the AAV was formulated when stored at about 5° C. In certain embodiments, the capsid titer in a pharmaceutical composition as described herein is maintained at least about 60% as compared to the capsid titer determined when the AAV was formulated, for at least about 6 months after the AAV was formulated when stored at about 25° C. In certain embodiments, the capsid titer in a pharmaceutical composition as described herein is maintained at least about 60% as compared to the capsid titer determined when the AAV was formulated, for at least about 2 months after the AAV was formulated when stored at about 40° C.

In certain embodiments, the pharmaceutical compositions of the present disclosure exhibit improved control over AAV (e.g., rAAV) vector aggregation (e.g., reduced accumulation of aggregates). In certain embodiments, the AAV (e.g., rAAV) vector aggregation can be determined by SEC. As determined by SEC, AAV aggregates are represented as the amount of high molecular weight species (HMWs) as a percentage of total protein (% HMWs) detected in a given composition. In certain embodiments, the pharmaceutical compositions of the present disclosure allow for the reduced accumulation of aggregates after a defined amount of time at a defined temperature. For example, the level of aggregates detected in a pharmaceutical composition described herein, represented as % HMWs in the composition, is less than about 5%, e.g., less than about 4%, less than about 3%, less than about 2%, less than about 1%, after a defined amount of time at a defined temperature. The defined amount of time can be, e.g., at least about 7 days, at least about 14 days, at least about 21 days, at least about 28 days, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 15 months, at least about 18 months, at least about 24 months, or more after the AAV was formulated. The defined temperature (e.g., the defined temperature at which the pharmaceutical composition is stored at) can be, e.g., about −80° C. to about 45° C., e.g., at about −80° C., about −40° C., about −30° C., about −20° C., about 0° C., about 4°−8° C., about 5° C., about 25° C., about 35° C., about 37° C., or about 45° C. For example, in certain embodiments, the level of aggregates detected in a pharmaceutical composition as described herein, represented as % HMWs in the composition, is less than about 1%, after at least about 9 months when stored at about −80° C. In certain embodiments the level of aggregates detected in a pharmaceutical composition as described herein, represented as % HMWs in the composition, is less than about 1%, after at least about 9 months when stored at about 5° C. In certain embodiments the level of aggregates detected in a pharmaceutical composition as described herein, represented as % HMWs in the composition, is less than about 2%, after at least about 6 months when stored at about 25° C. In certain embodiments the level of aggregates detected in a pharmaceutical composition as described herein, represented as % HMWs in the composition, is less than about 2%, after at least about 2 months when stored at about 40° C.

In certain embodiments, the pharmaceutical compositions of the present disclosure exhibit maintained levels of AAV vector purity. In certain embodiments, the AAV vector purity of a composition can be determined by CE-SDS. As determined by CE-SDS, the AAV vector purity of a composition is represented as the amount of AAV capsid proteins as a percentage of total protein (% purity) detected in a given composition. In certain embodiments, the pharmaceutical compositions of the present disclosure allow for maintained levels of AAV vector purity, represented as % purity, after a defined amount of time at a defined temperature. For example, the level of AAV vector purity of a pharmaceutical composition described herein, represented as % purity, is at least about 80%, e.g., about 80%, about 81%, about 82%, about 83%, about 84%, about 85%, about 86%, about 87%, about 88%, about 89%, about 90%, about 91%, about 92%, about 93%, about 94%, about 95%, about 96%, about 97% about 98%, about 99%, about 100% purity, after a defined amount of time at a defined temperature. The defined amount of time can be, e.g., at least about 7 days, at least about 14 days, at least about 21 days, at least about 28 days, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 15 months, at least about 18 months, at least about 24 months, or more after the AAV was formulated. The defined temperature (e.g., the defined temperature at which the pharmaceutical composition is stored at) can be, e.g., about −80° C. to about 45° C., e.g., at about −80° C., about −40° C., about −30° C., about −20° C., about 0° C., about 4°−8° C., about 5° C., about 25° C., about 35° C., about 37° C., or about 45° C. For example, in certain embodiments, the level of AAV vector purity of a pharmaceutical composition as described herein, represented as % purity, is at least about 90% after at least about 12 months when stored at about −80° C. In certain embodiments, the level of AAV vector purity of a pharmaceutical composition as described herein, represented as % purity, is at least about 90% after at least about 24 months when stored at about −80° C. In certain embodiments, the level of AAV vector purity of a pharmaceutical composition as described herein, represented as % purity, is at least about 90% after at least about 12 months when stored at about 5° C. In certain embodiments, the level of AAV vector purity of a pharmaceutical composition as described herein, represented as % purity, is at least about 90% after at least about 24 months when stored at about 5° C. In certain embodiments, the level of AAV vector purity of a pharmaceutical composition as described herein, represented as % purity, is at least about 90% after at least about 6 months when stored at about 25° C. In certain embodiments, the level of AAV vector purity of a pharmaceutical composition as described herein, represented as % purity, is at least about 90% after at least about 2 months when stored at about 40° C.

Additional methods for assessing the characteristics of a pharmaceutical composition comprising an AAV (e.g., rAAV) are demonstrated in the Examples presented below.

### VI. Routes of Administration and Methods of Use

In certain embodiments, the present disclosure provides a method for transducing a cell in a subject. The method generally comprises administering to the subject an effective amount of a pharmaceutical composition described herein.

The pharmaceutical compositions as described herein can be administered to a subject (e.g., a human subject) by any appropriate route, including, without limitation, intravenous, intraperitoneal, subcutaneous, intramuscular, intrathecal, intranasal, intracerebroventricular, topical or intradermal routes. In certain embodiments, the pharmaceutical compositions provided by the present disclosure are suitable for administration via intravenous injection or subcutaneous injection.

In certain embodiments, the pharmaceutical compositions provided by the present disclosure comprise an rAAV. The rAAV can comprise a transgene under the control of a TRE. Accordingly, in certain embodiments, the present disclosure provides methods for expressing a transgene in a cell in a subject, the method generally comprising administering to the subject an effective amount of a pharmaceutical composition comprising an rAAV as described herein, whereby the cell is transduced by the rAAV and the transgene is expressed. The transgene can encode a polypeptide and/or an RNA molecule, as described herein. Accordingly, in certain embodiments, the present disclosure provides methods for producing a polypeptide and/or an RNA molecule in a cell in a subject, the method generally comprising administering to the subject an effective amount of a pharmaceutical composition comprising an rAAV as described herein, whereby the cell is transduced by the rAAV and the polypeptide and/or an RNA molecule is produced.

The pharmaceutical compositions described herein can comprise an rAAV comprising an editing genome. rAAV comprising editing genomes can be used to edit the genome of a cell by homologous recombination of the editing genome with a homologous target locus in the cell. Accordingly, in certain embodiments, the present disclosure provides a method for editing a target locus in a genome of a cell in a subject, the method generally comprising administering to the subject an effective amount of a pharmaceutical composition comprising such an rAAV, whereby the cell is transduced by the rAAV and the target locus is edited.

Cells suitable for being transduced by the rAAV comprised within the pharmaceutical compositions described herein include, without limitation, cells of the blood, liver, heart, joint tissue, muscle, brain, kidney, or lung. In certain embodiments, the cell is a cell of the central nervous system or peripheral nervous system.

In another aspect, the invention provides an rAAV as disclosed herein for use in medicine. In another aspect, the invention provides an rAAV as disclosed herein for use as therapy. In another aspect, the invention provides an rAAV as disclosed herein for use as a medicament.

In another aspect, the present disclosure provides a method for the preparation of a pharmaceutical composition described herein, comprising the steps of mixing an adeno-associated virus (AAV), histidine, trehalose, and greater than about 150 mM sodium chloride.

In certain embodiments, the method further comprises one or more steps of freezing and thawing.

In certain embodiments, the method further comprises the step of storing the pharmaceutical composition at a temperature from about −80° C. to about 40° C. In some embodiments, the temperature is from about 2° C. to about 8° C. In some embodiments, the temperature is about 5° C. In some embodiments, the temperature is about 25° C. In some embodiments, the temperature is about 40° C.

### EXAMPLES

The following Examples are offered by way of illustration, and not by way of limitation.

### Example 1: Optimization of Adeno-Associated Virus Formulations

Several AAV vector formulations (Formulations 1-6) containing various concentrations of salt and stabilizing agent were generated. The components of these formulations are set forth in Table 1.

TABLE 1
Components of Formulations 1-6.

| | Formulation | | | | | |
|---|---|---|---|---|---|---|
| | 1 | 2 | 3 | 4 | 5 | 6 |
| Sodium | 20 | 20 | 20 | 20 | 20 | 20 |
| Phosphate | | | | | | |
| (mM) | | | | | | |
| Sodium | 175 | 150 | 200 | 175 | 175 | 175 |
| Chloride (mM) | | | | | | |
| Sucrose | 1 | 1 | 1 | — | — | — |
| (% w/v) | | | | | | |
| Trehalose | — | — | — | 1 | 3 | 5 |
| (% w/v) | | | | | | |
| Poloxamer | 0.03 | 0.03 | 0.03 | 0.03 | 0.03 | 0.03 |
| 188 (% w/v) | | | | | | |
| pH | 7.3 | 7.3 | 7.3 | 7.3 | 7.3 | 7.3 |

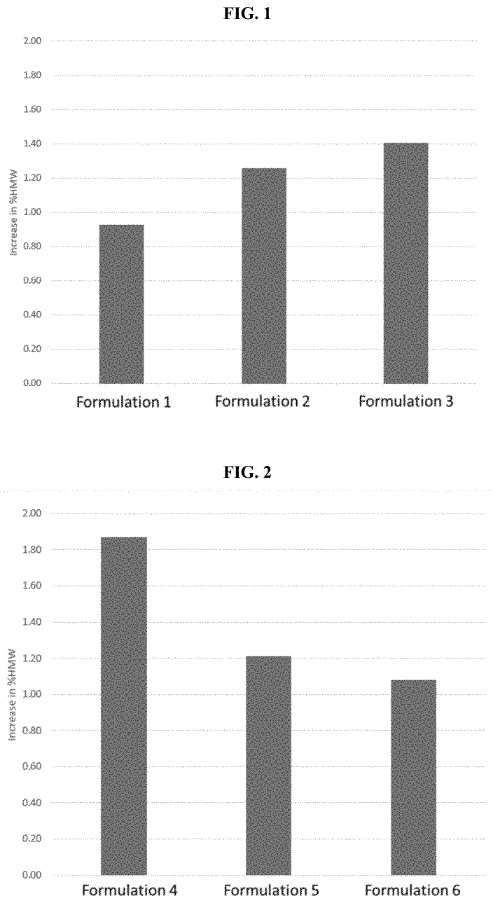

To assess the effect of salt concentration on AAV aggregation, AAV vectors were incubated in Formulations 1, 2, or 3 at 40° C. for 2 weeks, and the increase in percentage of AAV vector aggregation was determined. As shown in FIG. 1, Formulation 1 (comprising 175 mM sodium chloride) resulted in a lower percentage increase in AAV vector aggregation than Formulation 2 (comprising 150 mM sodium chloride) or Formulation 3 (comprising 200 mM sodium chloride).

To assess the effect of trehalose and sucrose on AAV aggregation, AAV vectors were incubated in Formulation 4, 5, or 6 at 40° C. for 2 weeks, and the increase in percentage AAV vector aggregation was determined. As shown in FIG. 2, Formulation 5 (comprising 3% (w/v) trehalose) resulted in a lower percentage increase in AAV vector aggregation than Formulation 4 (comprising 1% (w/v) trehalose). Formulation 6 (comprising 5% (w/v) trehalose) performed similarly to Formulation 5.

Several additional AAV vector formulations (Formulations 7-10) containing citrate or histidine buffers were generated. The components of these additional formulations are set forth in Table 2.

TABLE 2
Components of Formulations 7-10.

| | Formulation | | | |
|---|---|---|---|---|
| | 7 | 8 | 9 | 10 |
| Histidine (mM) | 20 | — | 20 | — |
| Citrate (mM) | — | 20 | — | 20 |
| Sodium Chloride | 175 | 175 | 175 | 175 |
| Sucrose (% w/v) | 1 | 1 | — | — |
| Trehalose (% w/v) | — | — | 3 | 3 |
| Poloxamer 188 (% w/v) | 0.03 | 0.03 | 0.03 | 0.03 |
| pH | 7.3 | 7.3 | 7.3 | 7.3 |

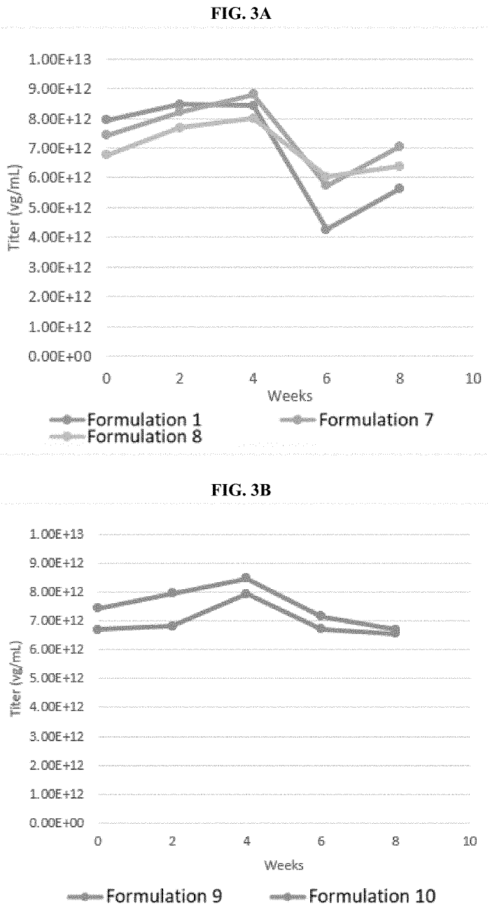

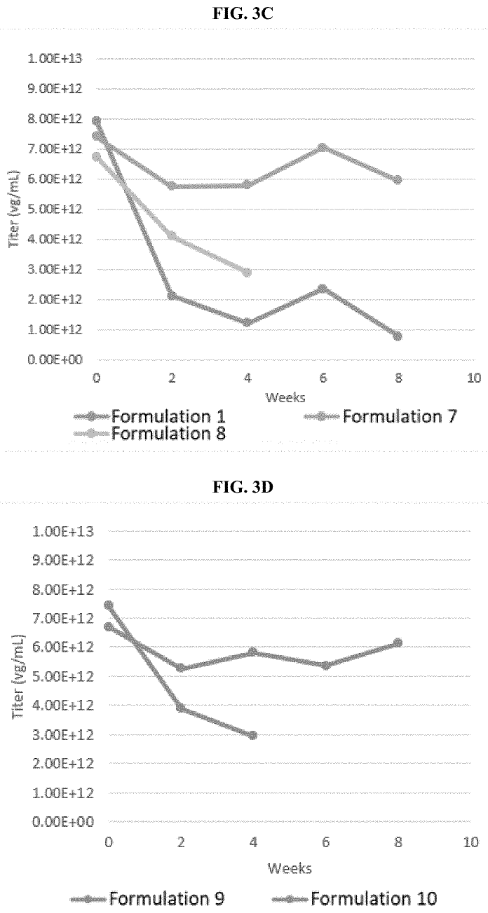

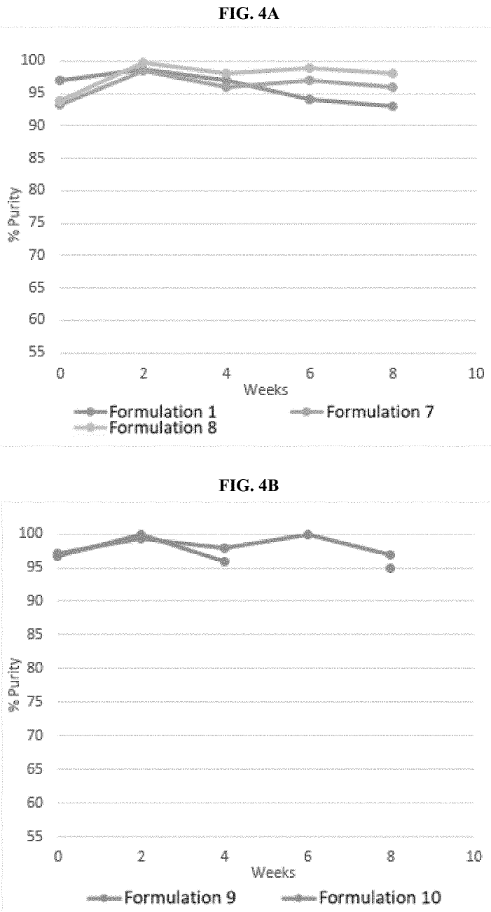

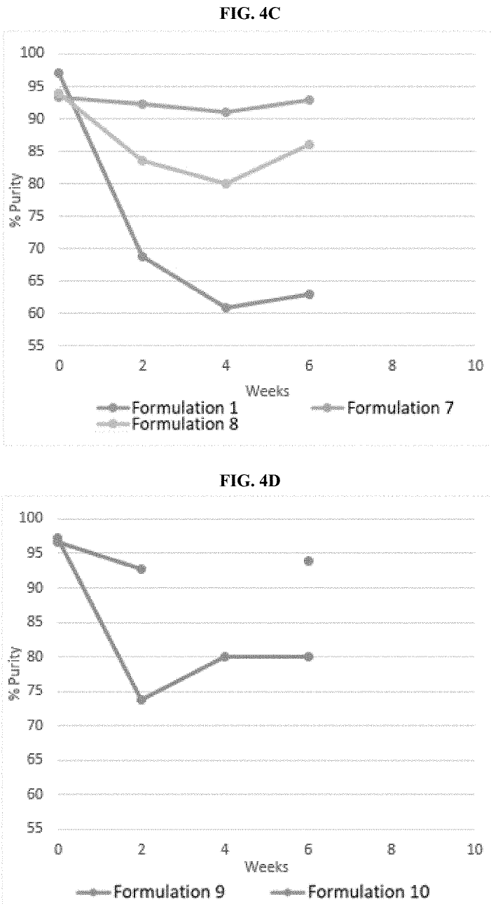

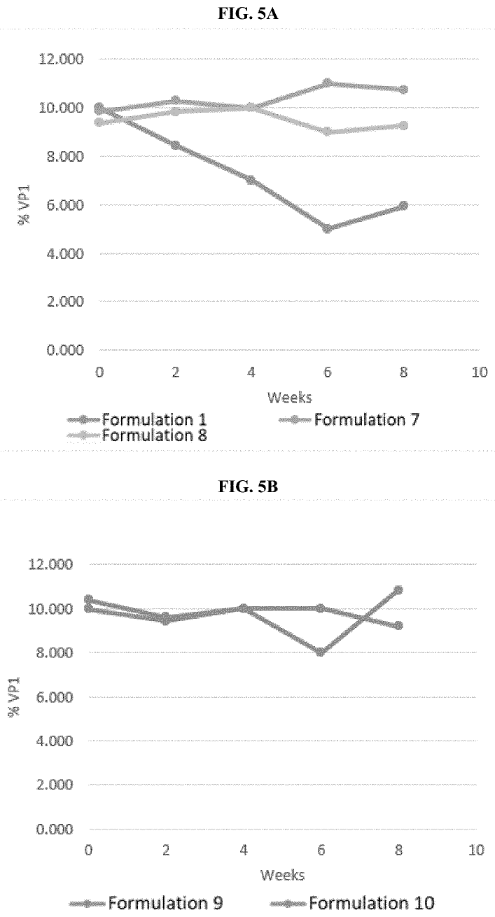

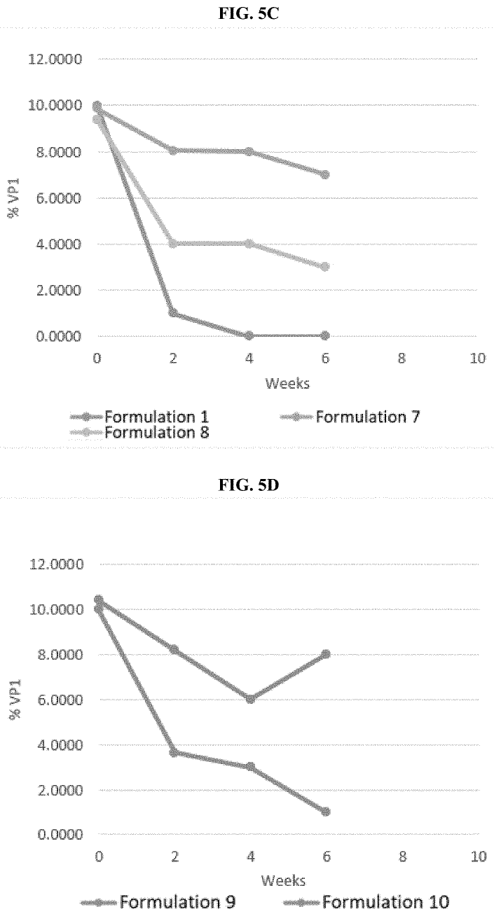

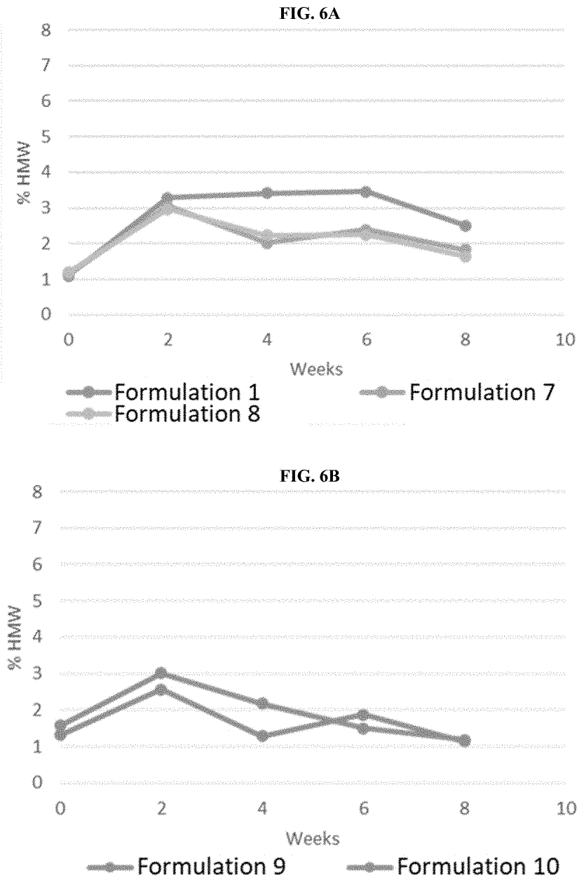

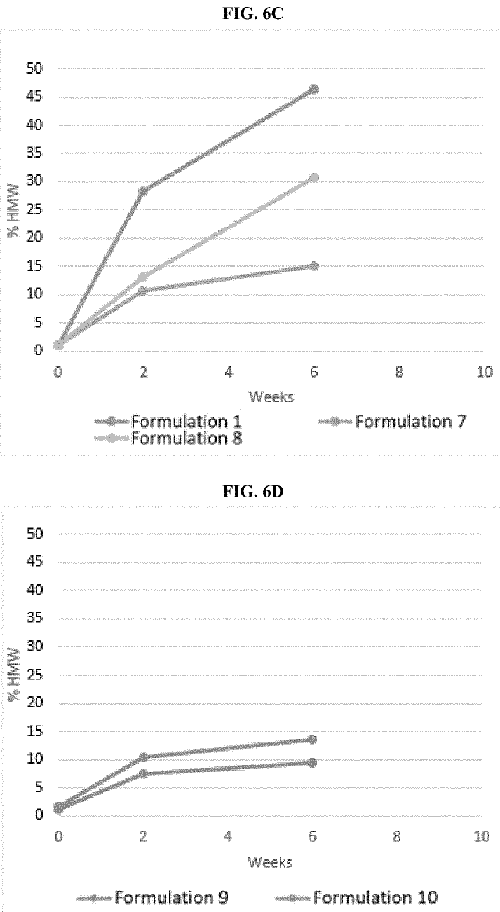

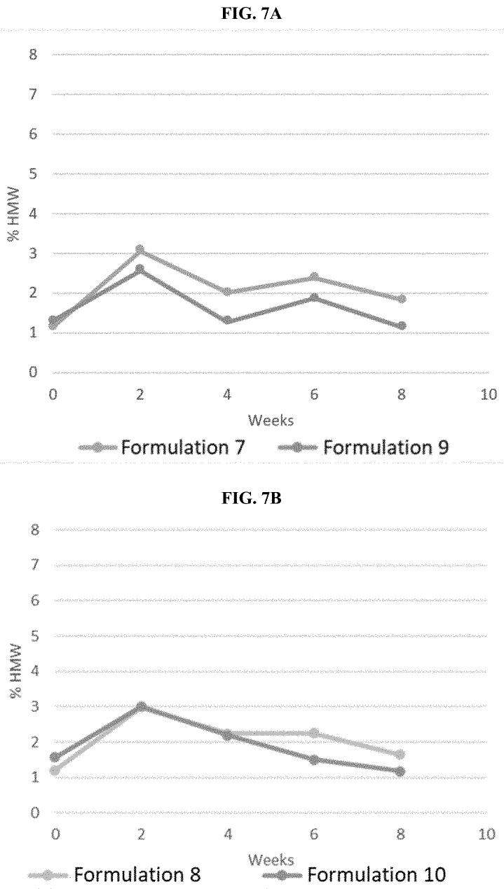

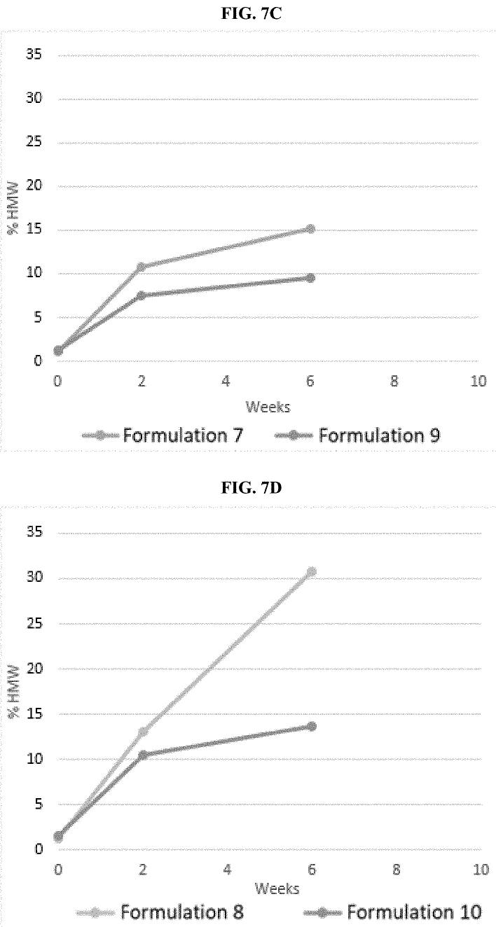

Formulations 1, 7, 8, 9, and 10 were tested for their ability to maintain viral genome titer (FIGS. 3A-3D), maintain total AAV vector purity (FIGS. 4A-4D), maintain VP1 capsid protein integrity (FIGS. 5A-5D), and control the level of AAV vector aggregate formation (FIGS. 6A-7D), over a specified period of time at a specified temperature.

Vector genome titers were determined using droplet digital PCR (ddPCR). As shown in FIGS. 3A-3D, formulations containing histidine (Formulations 7 and 9) were able to maintain vector genome titers over time when incubated at 25° C. (FIGS. 3A and 3B) and 40° C. (FIGS. 3C and 3D). Of the formulations containing 1% sucrose, the histidine formulation (Formulation 7) was able to maintain vector genome titers over time when incubated at both temperatures (FIGS. 3A and 3C). Of the formulations containing 3% trehalose, the histidine formulation (Formulation 9) was able to maintain vector genome titers over time when incubated at both temperatures (FIGS. 3B and 3D). When the formulations were incubated at 40° C., formulations containing histidine (Formulations 7 and 9) were able to maintain higher vector genome titers over time when compared to formulations containing citrate and sodium phosphate (FIGS. 3C and 3D).

AAV vector purity of the formulations and the ability of the formulations to retain VP1 capsid integrity (level of VP1 capsid protein integrity measured as a function of corrected area under the curve for VP1) was determined by CE-SDS analysis. As shown in FIGS. 4A-4D, formulations containing histidine (Formulations 7 and 9) were able to maintain AAV vector purity over time when incubated at 25° C. (FIGS. 4A and 4B) and 40° C. (FIGS. 4C and 4D). Of the formulations containing 1% sucrose, the histidine formulation (Formulation 7) was able to maintain AAV vector purity over time when incubated at both temperatures (FIGS. 4A and 4C). Of the formulations containing 3% trehalose, the histidine formulation (Formulation 9) was able to maintain AAV vector purity over time when incubated at both temperatures (FIGS. 4B and 4D). When the formulations were incubated at 40° C., formulations containing histidine (Formulations 7 and 9) were able to maintain higher AAV vector purity over time when compared to formulations containing citrate and sodium phosphate (FIGS. 4C and 4D). As shown in FIGS. 5A-5D, formulations containing histidine (Formulations 7 and 9) were able to retain VP1 capsid protein integrity over time when incubated at 25° C. (FIGS. 5A and 5B) and 40° C. (FIGS. 5C and 5D). Of the formulations containing 1% sucrose, the histidine formulation (Formulation 7) was able to retain VP1 capsid protein integrity over time when incubated at both temperatures (FIGS. 5A and 5C). Of the formulations containing 3% trehalose, the histidine formulation (Formulation 9) was able to retain VP1 capsid protein integrity over time when incubated at both temperatures (FIGS. 5B and 5D). When the formulations were incubated at 40° C., formulations containing histidine (Formulations 7 and 9) were able to retain higher VP1 capsid protein integrity over time when compared to formulations containing citrate and sodium phosphate (FIGS. 5C and 5D).

The level of AAV vector aggregates was determined as a function of the percentage of high molecular weight (HMW) species detected in each formulation using SEC. As shown in FIGS. 6A-6D, formulations containing histidine (Formulations 7 and 9) were able to better control the percentage of AAV vector aggregates formed over time when incubated at 25° C. (FIGS. 6A and 6B) and 40° C. (FIGS. 6C and 6D). Of the formulations containing 1% sucrose, the histidine formulation (Formulation 7) was able to better control the percentage of AAV vector aggregates formed over time when incubated at both temperatures (FIGS. 6A and 6C). Of the formulations containing 3% trehalose, the histidine formulation (Formulation 9) was able to better control the percentage of AAV vector aggregates formed over time when incubated at both temperatures (FIGS. 6B and 6D). When the formulations were incubated at 40° C., formulations containing histidine (Formulations 7 and 9) were able to better control the percentage of AAV vector aggregates formed over time when compared to formulations containing citrate and sodium phosphate (FIGS. 6C and 6D).

FIGS. 7A-7D show that the choice of sucrose or trehalose in histidine and citrate formulations has a large impact on the ability of the formulations to better control the percentage of AAV vector aggregates formed over time. Of the formulations containing histidine, the trehalose formulation (Formulation 9) was able to better control the percentage of AAV vector aggregates formed over time when incubated at 25° C. (FIG. 7A) and 40° C. (FIG. 7C). Of the formulations containing citrate, the trehalose formulation (Formulation 10) was able to better control the percentage of AAV vector aggregates formed over time when incubated at 25° C. (FIG. 7B) and 40° C. (FIG. 7D). As such, formulations containing trehalose (Formulations 9 and 10), irrespective of the buffering agent, outperformed sucrose formulations (Formulations 7 and 8) in terms of the ability of the formulations to better control the percentage of AAV vector aggregates formed over time.

The foregoing data suggests that AAV vector formulations comprising histidine buffer and trehalose can provide improved long term AAV vector stability to relative formulations comprising phosphate or citrate buffer and sucrose.

### Example 2: Long-Term Stability Studies

In order to further investigate the advantages of an AAV vector formulation containing histidine buffer and trehalose, a head-to-head comparison between Formulation 9 (comprising histidine buffer and trehalose) and Formulation 1 (comprising phosphate buffer and sucrose) was performed.

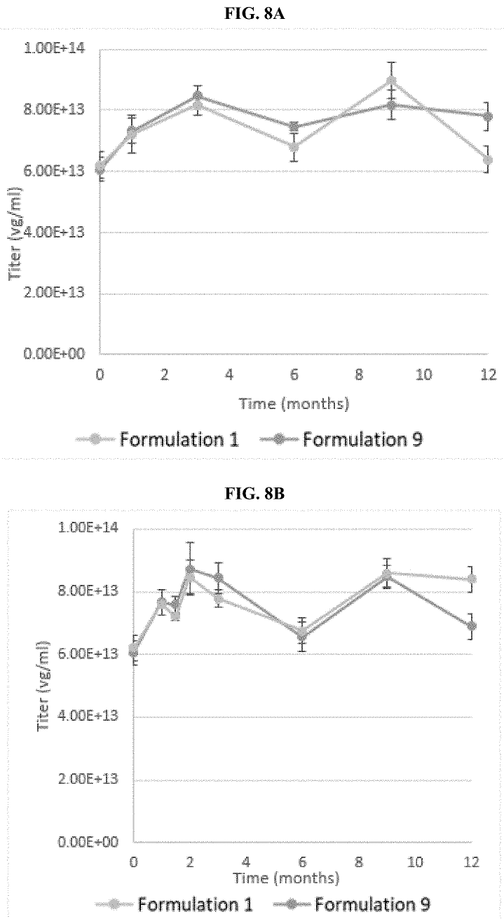

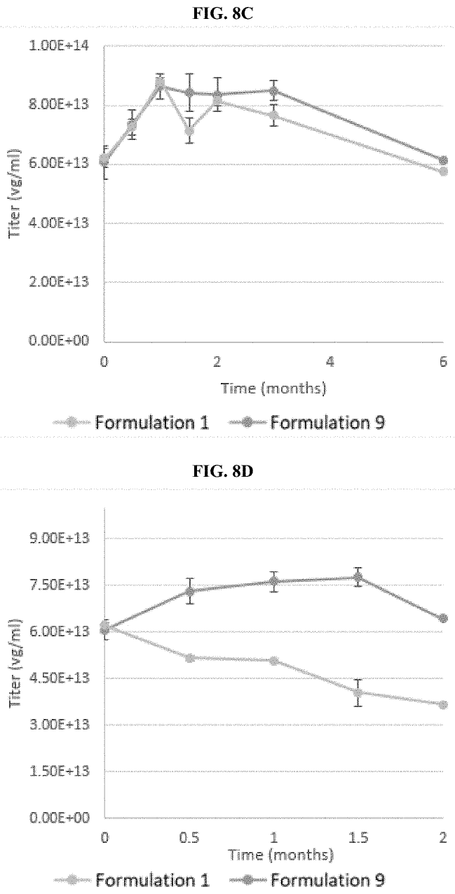

Formulations 1 and 9 incubated at various temperatures over time and AAV vector genome titers were determined using droplet digital PCR (ddPCR) (FIGS. 8A-8D). As shown in FIGS. 8A-8C, Formulations 1 and 9 were able to maintain vector genome titers to a similar extent over the time course studied, when incubated at −80° C. (FIG. 8A), 5° C. (FIG. 8B), and 25° C. (FIG. 8C). However, when the formulations were incubated at 40° C., Formulation 9 was able to maintain higher vector genome titers compared to Formulation 1 (FIG. 8D).

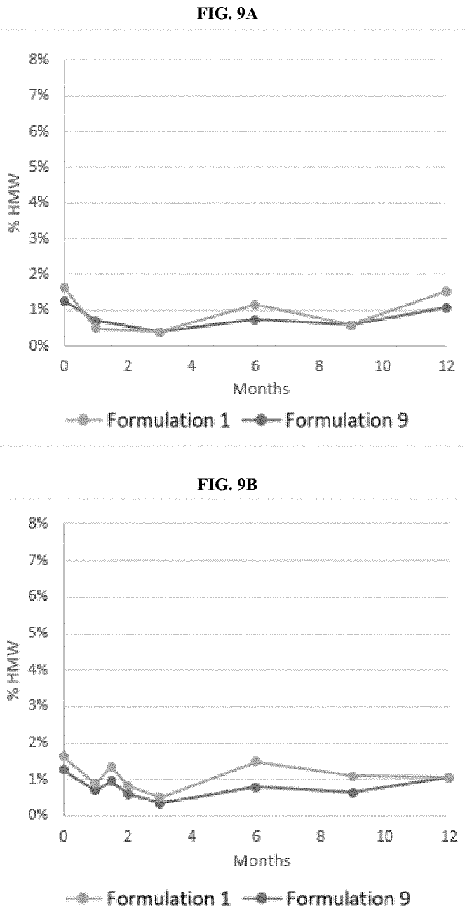

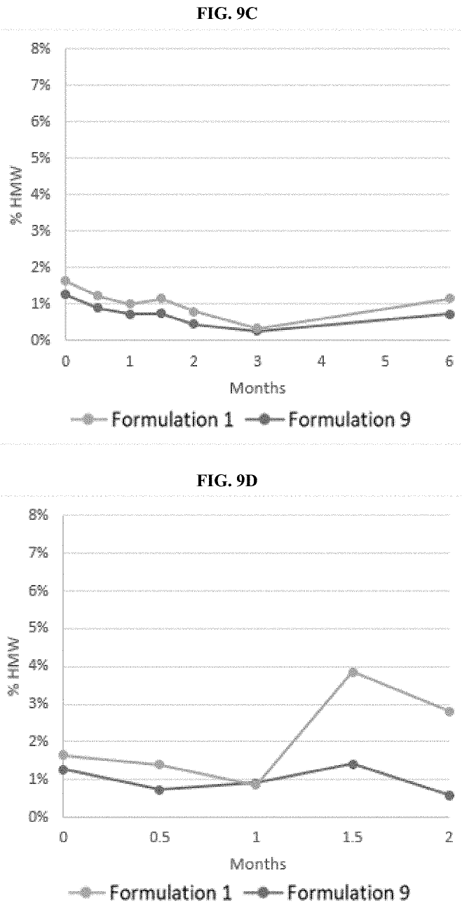

The level of AAV vector aggregates in Formulations 1 and 9 incubated at various temperatures was determined as a function of the percentage of high molecular weight (HMW) species detected using SEC (FIGS. 9A-9D). As shown in FIGS. 9A-9C, the percentage of aggregates formed over the time course studied in Formulations 1 and 9 was similar when incubated at −80° C. (FIG. 9A), 5° C. (FIG. 9B), and 25° C. (FIG. 9C). However, the percentage of aggregates formed after 2 months in Formulation 9 at 40° C. was significantly less compared to Formulation 1 (FIG. 9D).

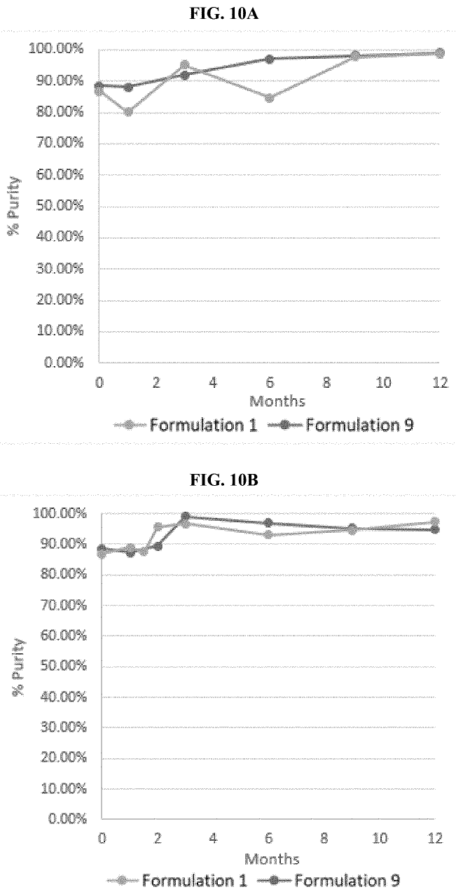

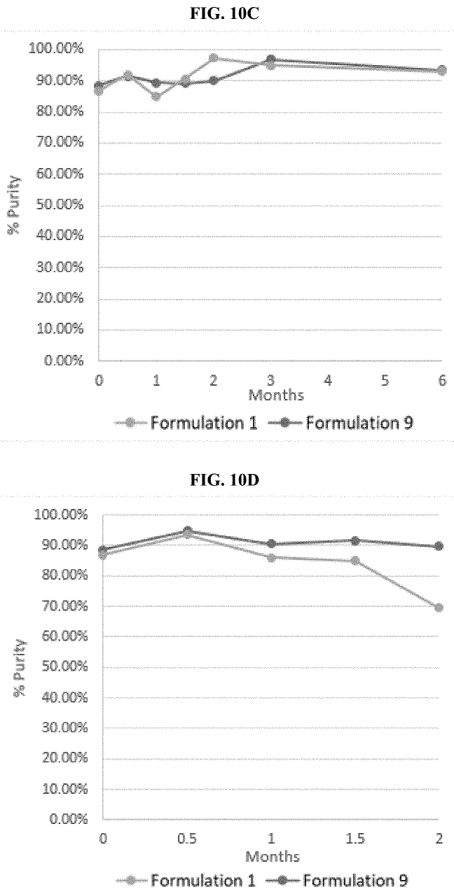

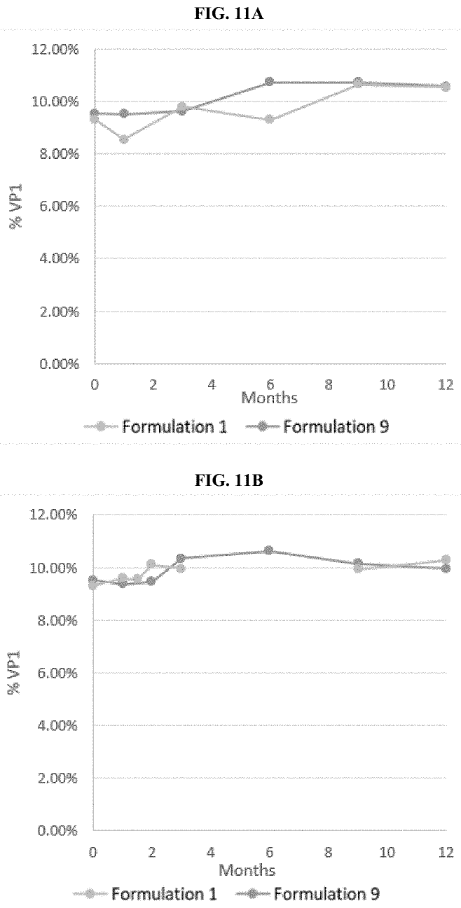

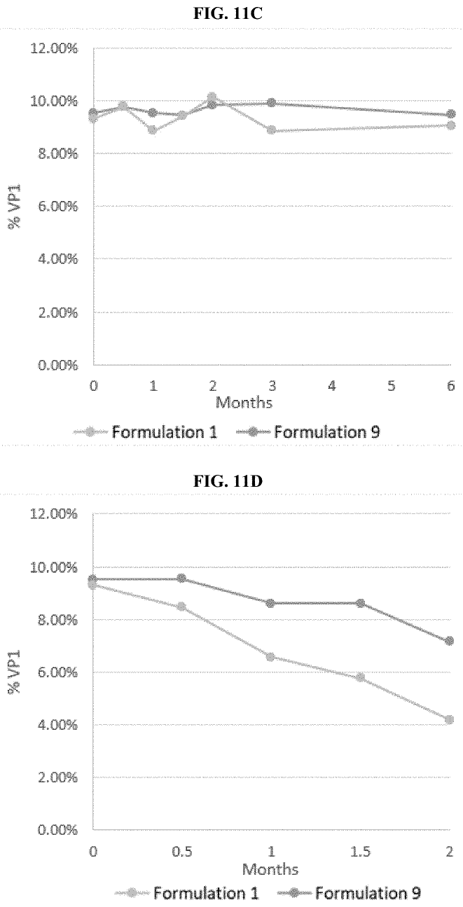

AAV vector purity of the formulations and the ability of the formulations to retain VP1 capsid protein integrity (level of VP1 capsid protein integrity measured as a function of corrected area under the curve for VP1) was determined by CE-SDS analysis of AAV vectors incubated at various temperatures over the time course studied in Formulations 1 and 9 (FIGS. 10A-11D). As shown in FIGS. 10A-10C, both Formulations 1 and 9 were able to maintain a similar level of AAV vector purity over time, when incubated at −80° C. (FIG. 10A), 5° C. (FIG. 10B), and 25° C. (FIG. 10C). However, when the formulations were incubated at 40° C., Formulation 9 was able to maintain higher AAV vector purity as compared to Formulation 1 (FIG. 10D). As shown in FIGS. 11A-11C, Formulations 1 and 9 were able to retain a similar level of VP1 capsid protein integrity over time, when incubated at −80° C. (FIG. 11A), 5° C. (FIG. 11B), and 25° C. (FIG. 11C). However, when the formulations were incubated at 40° C., Formulation 9 was able to retain a higher level of VP1 capsid protein integrity compared to Formulation 1 (FIG. 11D).

The foregoing data confirmed that AAV vector formulations comprising histidine buffer and trehalose can provide improved long term AAV vector stability relative to more traditional phosphate buffered formulations.

### Example 3: Long-Term Stability Studies—Temperature

In order to assess the stability of AAV vector formulations comprising histidine buffer and trehalose, Formulation 9 was incubated at various temperatures. Vector genome titers, capsid titers, AAV aggregation, AAV vector purity, capsid protein integrity, and AAV vector potency were measured over time.

Clade F Capsid material was received in Formulation 9 as Drug Substance. The Drug Substance was 0.2 μm filtered under a Biosafety Cabinet and vialed into 0.5 mL Crystal Zenith COP vials to make the Drug Product. The Drug Product was then placed on stability at −80° C., 5° C., and 25° C. At stability timepoints, the vials were removed from their respective storage conditions and subjected to testing of VG titers by ddPCR, capsid titers by ELISA, AAV vector aggregates by SEC, AAV vector purity by Capillary Electrophoresis (CE), VP1 content by CE, and AAV potency by relative gene expression. A subset of samples were evaluated for sub-visible particles using the Aura Halo system following USP <788> guidelines.

The level of VG titers, capsid titers, AAV % HMW, VP1 capsid protein integrity, and AAV vector purity were determined as described in Example 1. The level of AAV vector potency was determined by relative gene expression. Subvisible particulate counts were measured in terms of particle counts/mL.

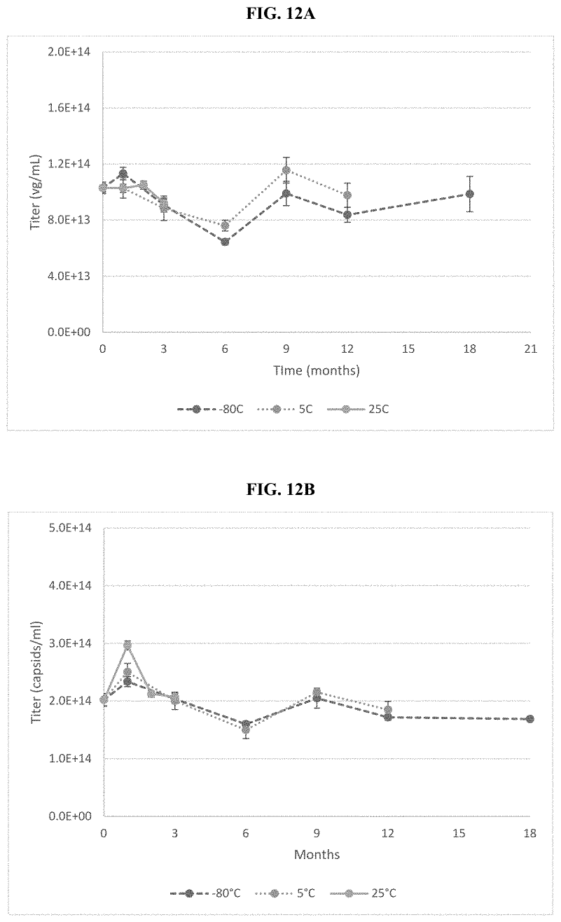

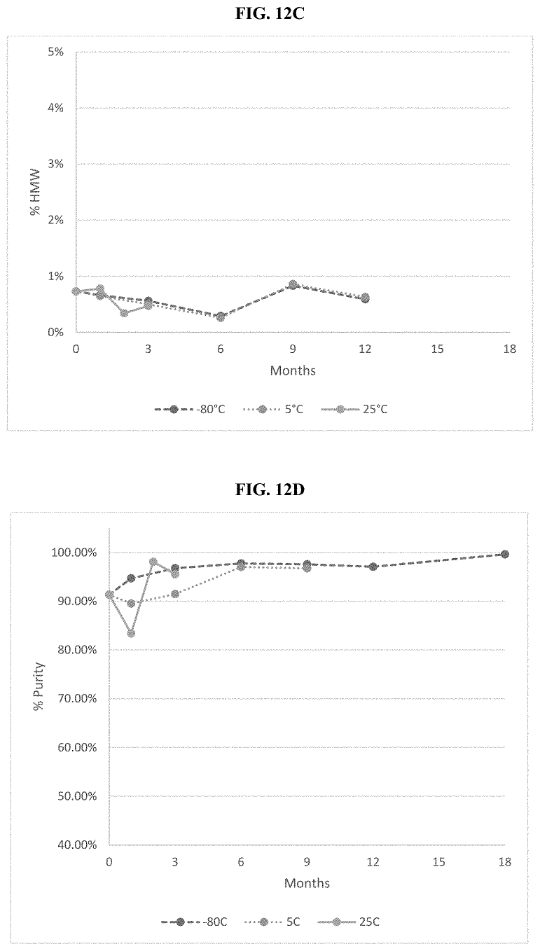

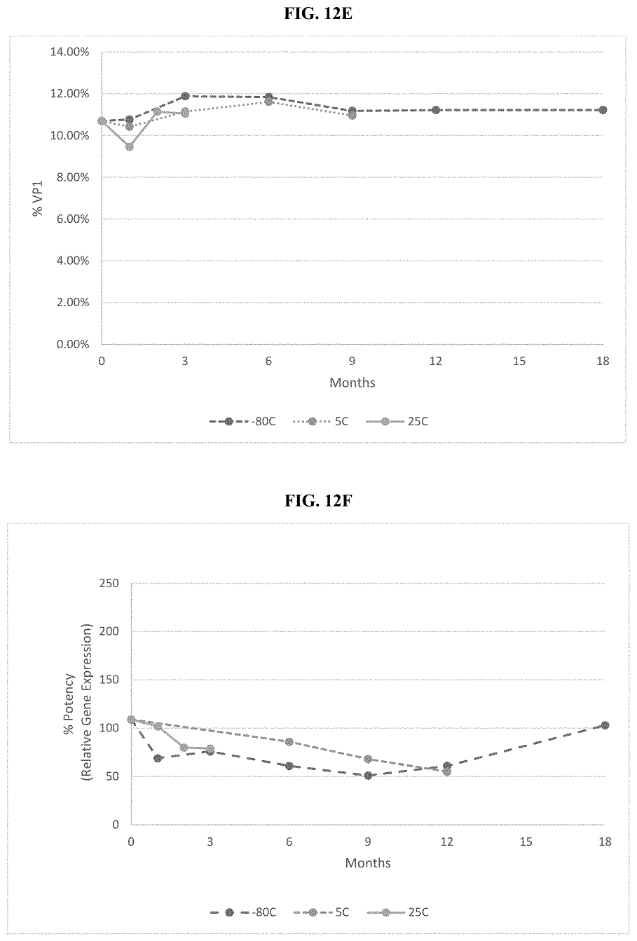

As shown in FIG. 12, Formulation 9 was able to maintain the level of vector genome titers (FIG. 12A), capsid titers (FIG. 12B), AAV vector aggregates (FIG. 12C), AAV vector purity (FIG. 12D), VP1 capsid protein integrity (FIG. 12E), and AAV vector potency (FIG. 12F) over time when incubated at −80° C., 5° C., and 25° C. Stability was maintained at −80° C. for 18 months, 5° C. for 12 months, and 25° C. for 3 months.

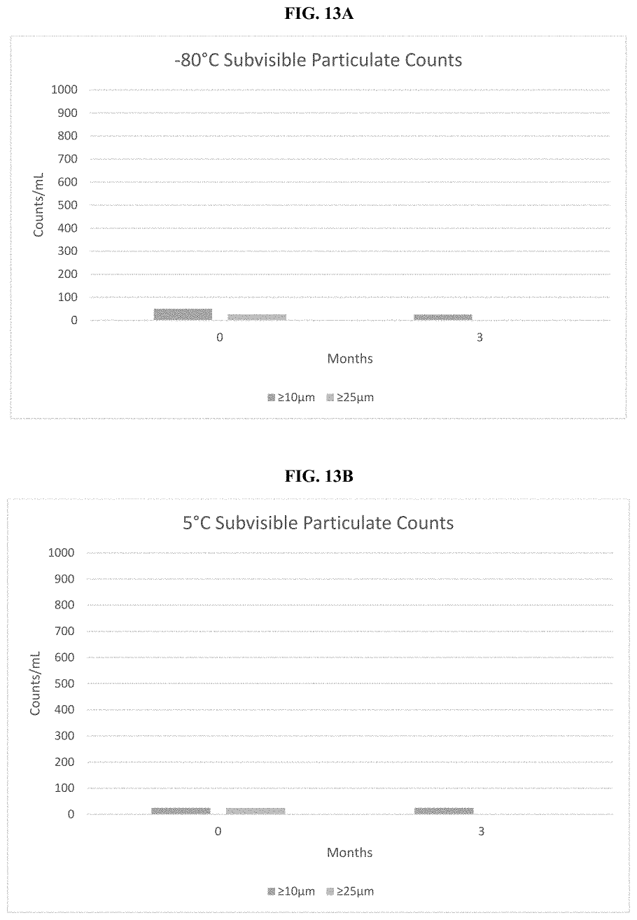

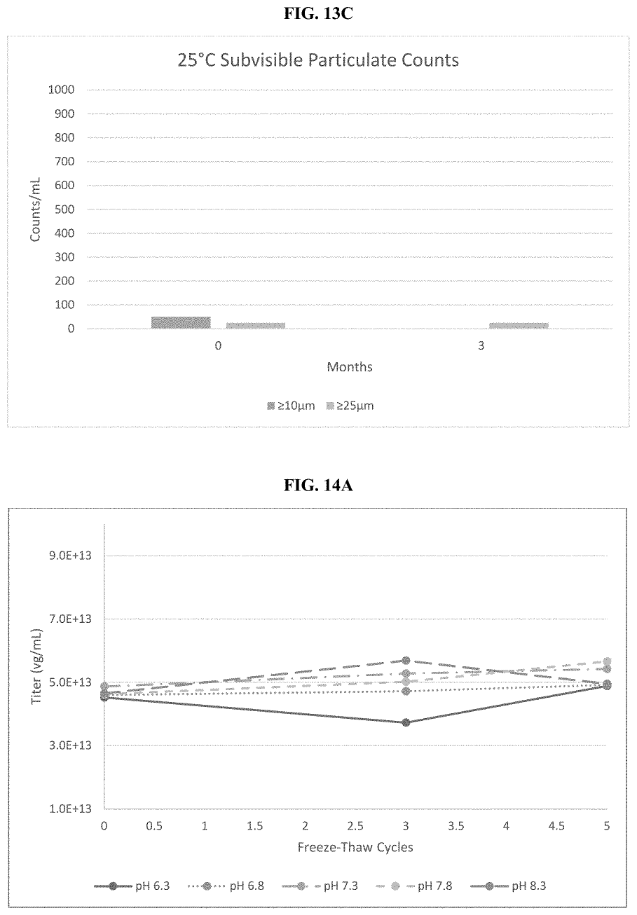

As shown in FIG. 13, Formulation 9 was able to maintain and keep the level of subvisible particulate counts over time below the USP 787 Guidance of >10 μm: <6000 particles/mL≥25 μm: <600 particles/mL when incubated at −80° C. (FIG. 13A), 5° C. (FIG. 13B) and 25° C. (FIG. 13C).

The foregoing data suggests that AAV vector formulations comprising histidine buffer and trehalose can provide improved long term AAV vector stability across various thermal stresses.

### Example 4: Product Stability Studies—Freeze-Thaw Cycles

In order to further assess the stability of AAV vector formulations comprising histidine buffer and trehalose, Formulation 9 was incubated at various pH values and subjected to multiple freeze-thaw cycles. The level of AAV vector potency, vector genome titers, capsid titers, percentage aggregation of AAV vectors, AAV vector purity, VP1 capsid protein integrity, and AAV potency were measured.

Clade F Capsid material was received in Formulation 9 as Drug Substance. The Drug Substance was initially titered to obtain T:0 vector genome titer. Once titer was received, 0.3 mL of Drug Substance was filled into 0.5 mL Crystal Zenith COP vials under a Biosafety Cabinet (BSC). Once vials were filled, they were stoppered under BSC, capped with aluminium seal and labelled. Once sealed, one vial was subjected to testing at 5° C. to obtain Ox Freeze-thaw/never frozen sample. All other samples were frozen at 80° C. Once samples were completely frozen, they were placed at 5° C. to thaw until no ice was present in the sample. This process was then repeated as many times as necessary to obtain the appropriate number of freeze-thaws.

Vector genome titers, capsid titers, AAV vector aggregates, AAV vector purity, and VP1 capsid protein integrity were determined as described in Example 1. The level of AAV vector potency was determined as described in Example 3.

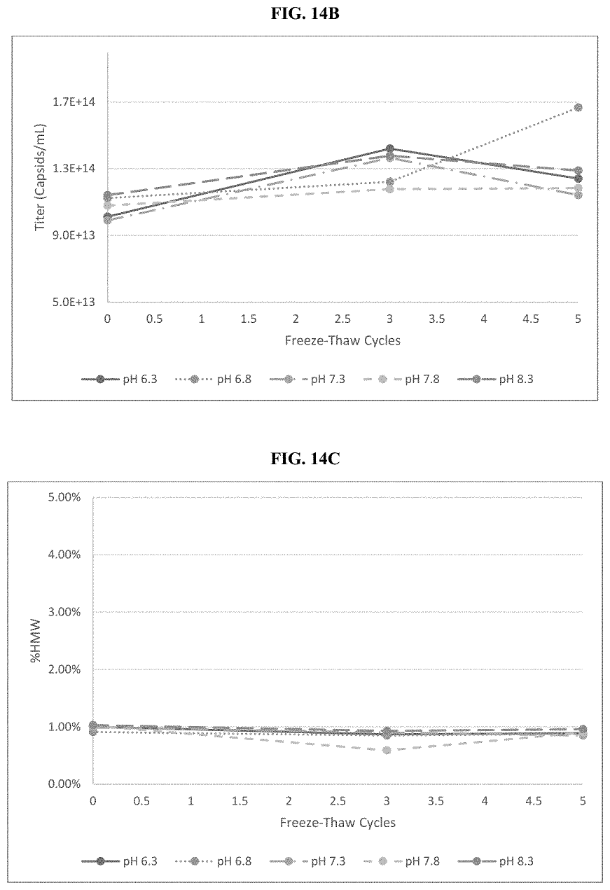

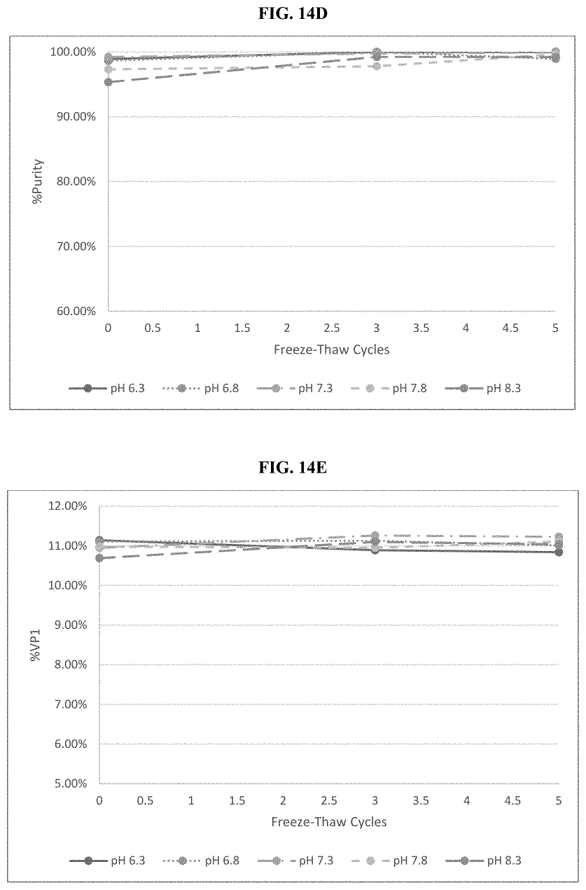

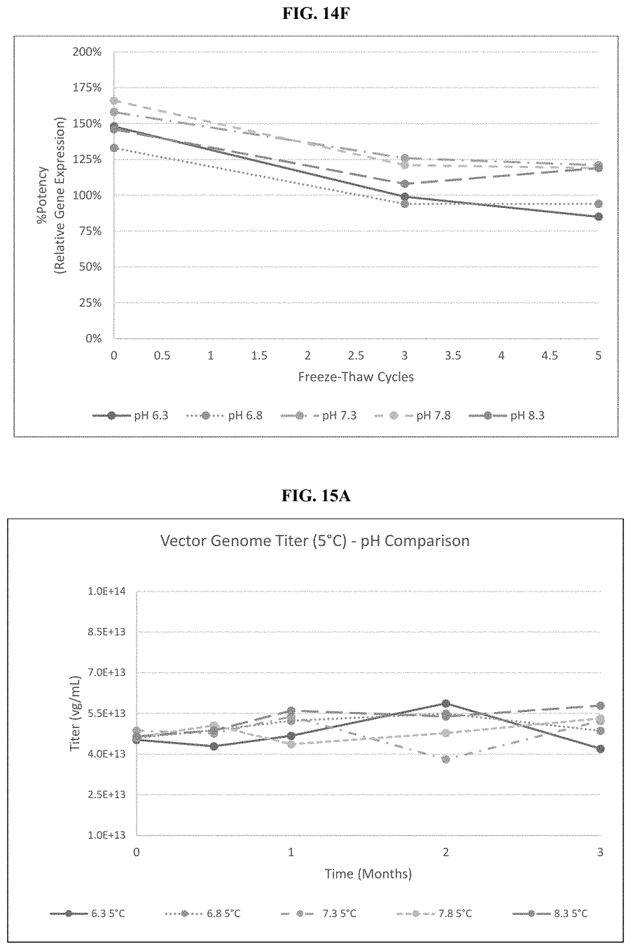

As shown in FIG. 14, Formulation 9 was able to maintain the level of vector genome titers (FIG. 14A), capsid titers (FIG. 14B), AAV vector aggregates (FIG. 14C), AAV vector purity (FIG. 14D), and VP1 capsid protein integrity (FIG. 14E), and AAV vector potency (FIG. 14F) across multiple freeze-thaw cycles at a range of pH values (6.3 to 8.3).

The foregoing data suggests that AAV vector formulations comprising histidine buffer and trehalose can provide improved AAV vector stability across multiple freeze-thaw cycles at a range of pH values.

### Example 5: Long-Term Stability Studies—pH

In order to further assess the stability of AAV vector formulations comprising histidine buffer and trehalose, Formulation 9 was incubated at various pH values, and at different temperatures. The level of vector genome titers, capsid titers, AAV vector aggregates, AAV purity, and VP1 capsid protein integrity were measured.

This study was started by first prepping the Formulation 9 buffer (20 mM Histidine, 175 mM Sodium Chloride, 3% (w/v %) Trehalose, 0.03% Poloxamer 188). The buffers were then adjusted to the target pH (6.3, 6.8, 7.3 (control), 7.8, 8.3). Clade F Capsid material was received as Drug Substance (DS) in standard Formulation 9 buffer. DS was buffer exchanged into new Formulation 9 buffers at the different pH values by spin concentration in a centrifuge. Five separate 15 mL centrifuge filtration tubes were filled with 15 mL of DS in Formulation 9 buffer. DS concentrated until approximately 5 mL of DS remained in the centrifuge filtration tube. 10 mL of designated pH buffer was then added to appropriate filtration tubes. This process was repeated 3× to ensure thorough buffer exchange took place. A 100 μL aliquot was taken from each pH sample to measure and confirm appropriate pH was reached. Once buffer exchange was complete, the material was taken up into a 20 mL syringe with a 21G needle, then filtered through a 0.2 μm filtered into a new sterile 15 mL conical tube to create the Drug Product (DP). Each arm was then filled into 15 labeled sterile 2 mL CZ vials containing 1 mL of Drug Product each using a 1 mL pipette. Vials were then stoppered and then placed in its designated sample box and placed at appropriate storage for stability. Samples were removed from chambers at designated time points and stored at −80° C. prior to analytical testing, including the level of vector genome titers, capsid titers, AAV vector aggregates, AAV vector purity, and level of AAV potency.

The level of vector genome titers, capsid titers, AAV vector aggregates, AAV vector purity, and VP1 capsid protein integrity were determined as described in Example 1. The level of AAV potency was determined as described in Example 3.

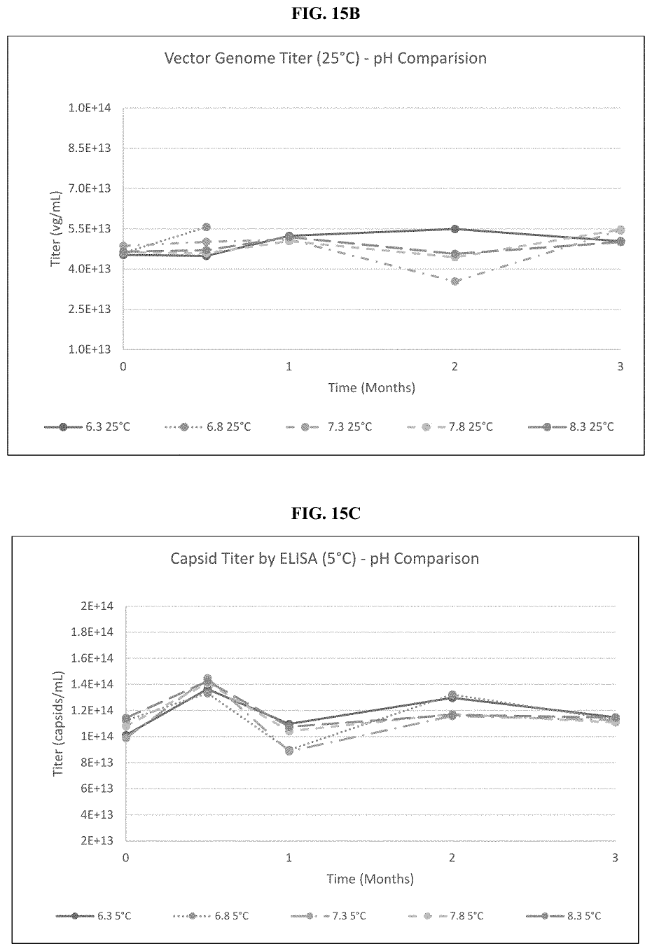

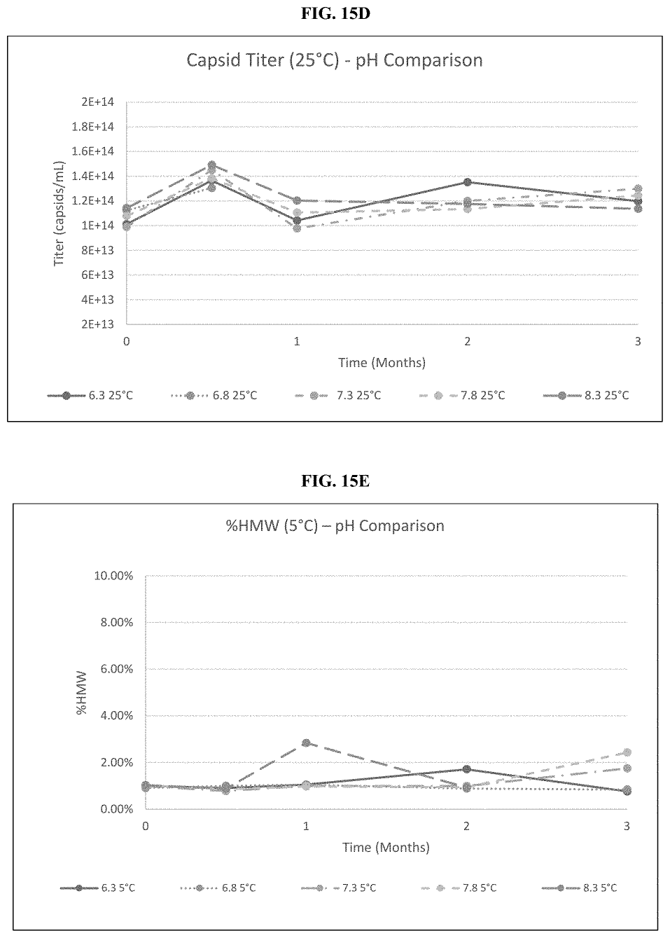

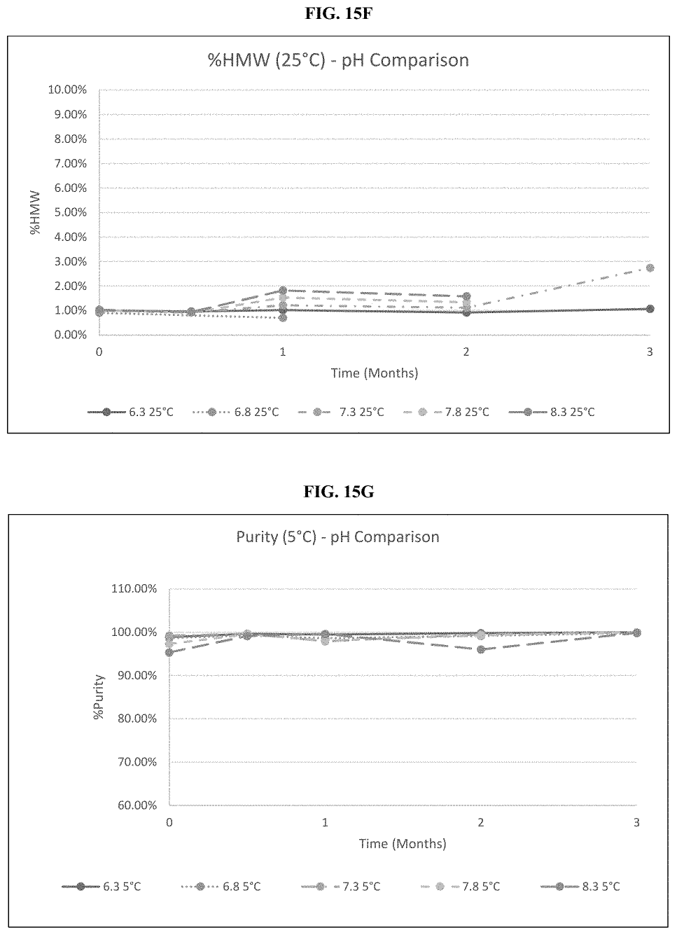

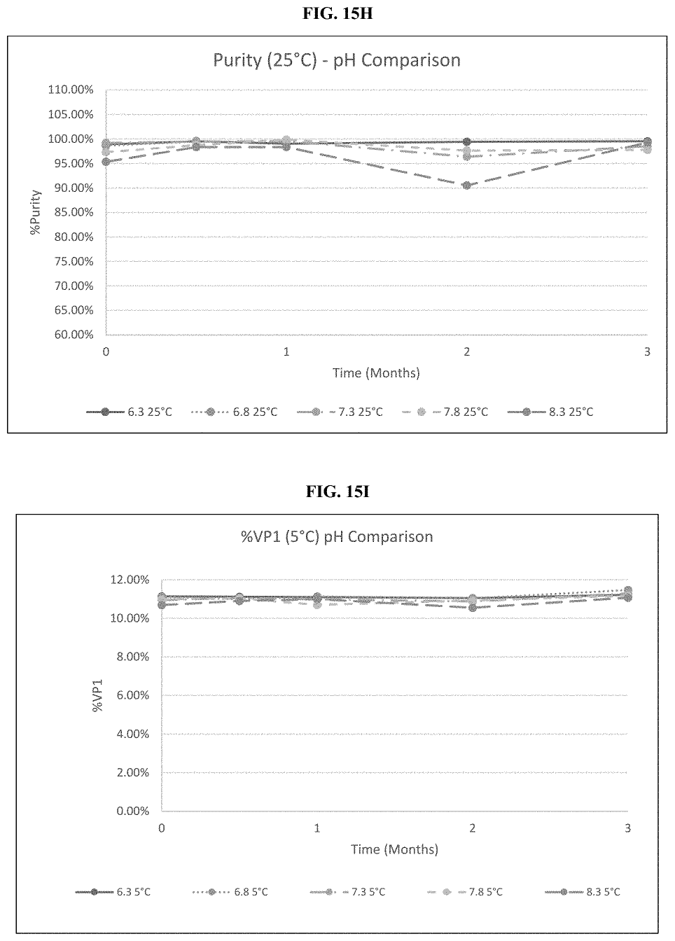

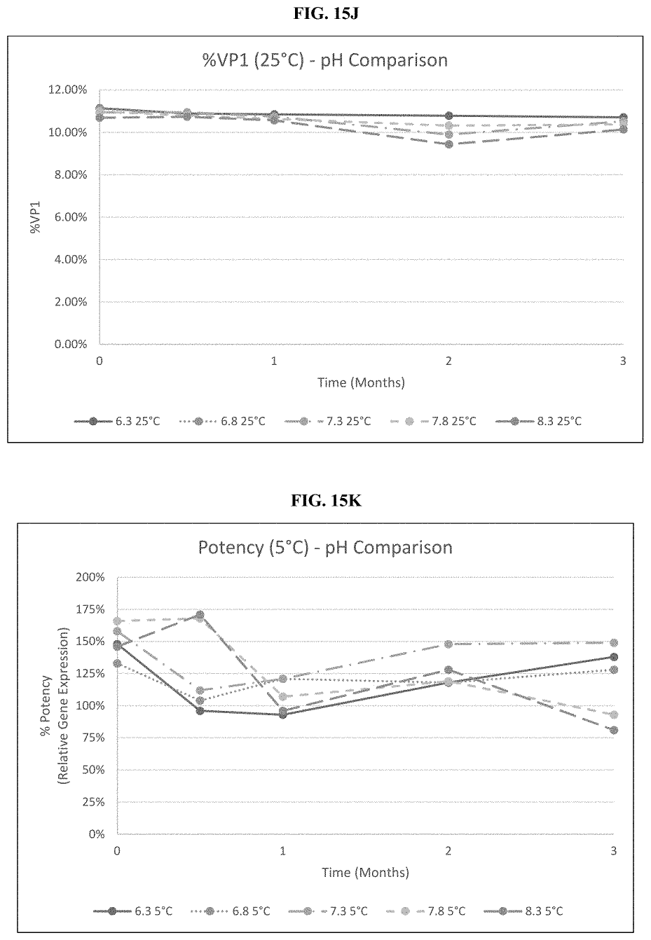

As shown in FIG. 15, Formulation 9 was able to maintain the level of vector genome titers (FIGS. 15A-15B), capsid titers (FIGS. 15C-15D), AAV vector aggregates (FIGS. 15E-15F), AAV vector purity (FIGS. 15G-15H), and VP1 capsid protein integrity (FIGS. 15I-15J) at a range of pH values (6.3-8.3) at 5° C. (FIGS. 15A, C, E, G, I) and 25° C. (FIGS. 15B, D, F, H, J).

The foregoing data suggests that AAV vector formulations comprising histidine buffer and trehalose can provide improved long term AAV vector stability across a range of pH values and temperatures.

### Example 6: High Titer Achievement

To assess the stability of AAV vector formulations comprising histidine buffer and trehalose across various AAV titers, the level of AAV vector aggregation was determined at different concentrations of a capsid drug product in Formulation 9 prepared over time.

Clade F Capsid Drug Product (DP) was received at 4.56E13 vg/mL in Formulation 9. To prepare higher concentration samples, 40 mL of DP was concentrated to 4 mL using a centrifuge filtration tubes. The 4 mL of 10×concentrated material was added to a new centrifuge tube and material was concentrated while stopping periodically to inspect the volume and material. Notes were taken at each stopping point and a 150 μL aliquot was removed and set aside for analytical testing. This was then repeated for all the following targets. Samples were submitted for analytical testing, including the level of vector genome titers, capsid titers, AAV vector aggregates, AAV vector purity, and level of AAV potency.

Vector genome titers, capsid titers, AAV vector aggregates, AAV vector purity, and VP1 capsid protein integrity were determined as described in Example 1. The level of AAV potency was determined as described in Example 3. In addition, the level of AAV vector aggregates was determined as a function of diameter on Dynamic Light Scattering (DLS) or Polydispersity Index (PDI).

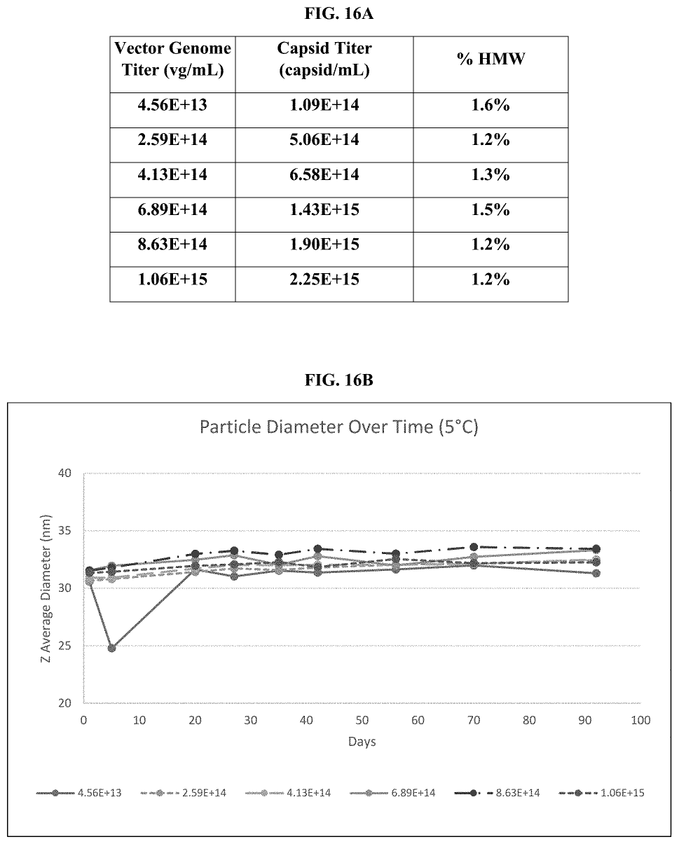

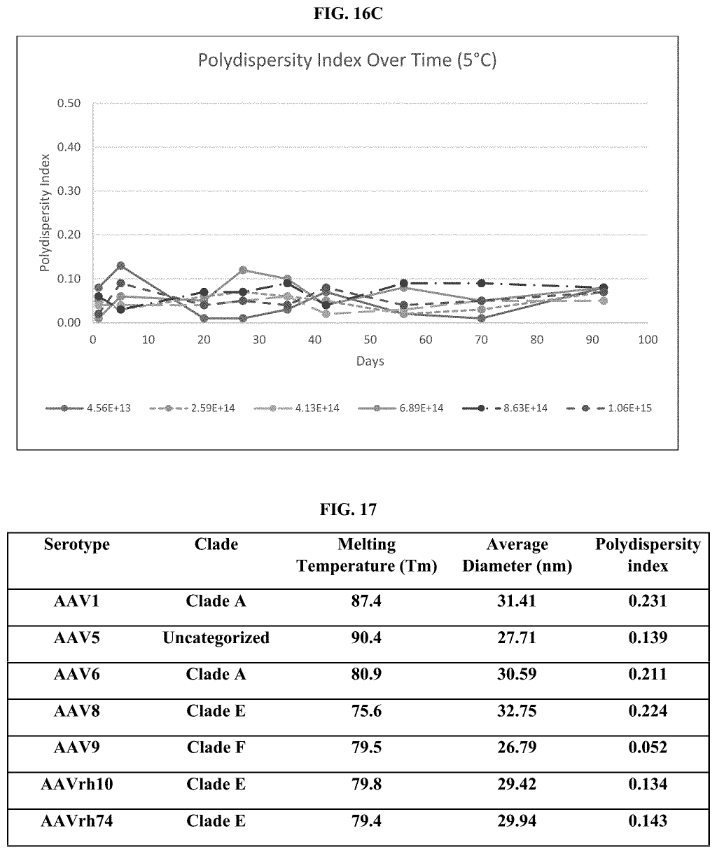

As shown in FIGS. 16A-16C, Formulation 9 was able to maintain the level of AAV vector aggregates as a function of high molecular weight species (% HMW) (FIG. 16A) and as a function of diameter on DLS or PDI (FIGS. 16B-C) across a range of vector genome titers and capsid titers.

The foregoing data suggests that AAV vector formulations comprising histidine buffer and trehalose can provide improved long term AAV vector stability at high AAV titers.

### Example 7: Alternative Capsid Serotypes

To assess whether the advantages of an AAV vector formulation containing histidine buffer and trehalose extend across alternative capsid serotypes, the stability of various capsid serotypes in Formulation 9 was measured.

Capsids from different Clades were received as an affinity product and buffer exchanged into Formulation 9 using centrifuge filtration tubes. Polydispersity and average particle diameter were evaluated by Static Light Scattering (SLS) and DLS. To measure melting temperature, a thermal ramp coupled with SLS measurements at each temperature ramp was used.

As shown in FIG. 17, Formulation 9 was able to maintain stability in terms of melting temperature and AAV aggregation across multiple AAV capsid serotypes.

The foregoing data suggests that AAV vector formulations comprising histidine buffer and trehalose can provide improved long term AAV vector stability across multiple AAV capsid serotypes.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

Further embodiments of the invention are set out in the following clauses:

Clause 1. A pharmaceutical composition comprising:

(a) an adeno-associated virus (AAV);

(b) histidine;

(c) trehalose; and

(d) greater than about 150 mM sodium chloride.

Clause 2. The pharmaceutical composition of clause 1, comprising about 5 mM to about 50 mM histidine.

Clause 3. The pharmaceutical composition of clause 1 or 2, comprising about 20 mM histidine.

Clause 4. The pharmaceutical composition of any preceding clause, comprising about 1% (w/v) to about 10% (w/v) trehalose.

Clause 5. The pharmaceutical composition of clause 4, comprising about 1% (w/v) trehalose.

Clause 6. The pharmaceutical composition of clause 4, comprising about 3% (w/v) trehalose.

Clause 7. The pharmaceutical composition of clause 4, comprising about 5% (w/v) trehalose.

Clause 8. The pharmaceutical composition of any one of clauses 1-7, comprising no more than about 200 mM sodium chloride.

Clause 9. The pharmaceutical composition of any one of clauses 1-7, comprising about 175 mM sodium chloride.

Clause 10. The pharmaceutical composition of any one of clauses 1-7, comprising about 200 mM sodium chloride.

Clause 11. The pharmaceutical composition of any preceding clause, further comprising about 0.01% (w/v) to about 0.05% (w/v) Poloxamer 188.

Clause 12. The pharmaceutical composition of clause 11, comprising about 0.03% (w/v) Poloxamer 188.

Clause 13. The pharmaceutical composition of clause 1, comprising:

(a) an adeno-associated virus (AAV);

(b) about 20 mM histidine;

(c) about 3% (w/v) trehalose;

(d) about 0.03% (w/v) Poloxamer 188; and

(e) about 175 mM sodium chloride.

Clause 14. The pharmaceutical composition of any preceding clause, wherein the pH of the pharmaceutical composition is about 7.3.

Clause 15. The pharmaceutical composition of any preceding clause, comprising at least about 1e13 vg/mL of the AAV.

Clause 16. The pharmaceutical composition of any preceding clause, comprising about 1e13 vg/mL to about 6e15 vg/mL of the AAV.

Clause 17. The pharmaceutical composition of clause 16, comprising about 2e13 vg/mL of the AAV.

Clause 18. The pharmaceutical composition of clause 16, comprising about 6e13 vg/mL of the AAV.

Clause 19. The pharmaceutical composition of clause 16, comprising about 8e13 vg/mL of the AAV.

Clause 20. The pharmaceutical composition of clause 16, comprising about 1e14 vg/mL of the AAV.

Clause 21. The pharmaceutical composition of clause 16, comprising about 2e14 vg/mL of the AAV.

Clause 22. The pharmaceutical composition of any preceding clause, comprising at least about 1e15 vg/mL of the AAV.

Clause 23. The pharmaceutical composition of any preceding clause, wherein the AAV is a recombinant AAV (rAAV) comprising an rAAV genome comprising a transgene.

Clause 24. The pharmaceutical composition of clause 23, wherein the transgene encodes a polypeptide.

Clause 25. The pharmaceutical composition of clause 23, wherein the transgene encodes an miRNA, shRNA, siRNA, antisense RNA, gRNA, antagomir, miRNA sponge, RNA aptazyme, RNA aptamer, lncRNA, ribozyme, or mRNA.

Clause 26. The pharmaceutical composition of clause 23, wherein the transgene encodes a protein selected from the group consisting of phenylalanine hydroxylase (PAH), glucose-6-phosphatase (G6Pase), iduronate-2-sulfatase (12S), arylsulfatase A (ARSA), and frataxin (FXN).

Clause 27. The pharmaceutical composition of any one of clauses 23-26, wherein the rAAV genome further comprises a transcriptional regulatory element operably linked to the transgene.

Clause 28. The pharmaceutical composition of clause 27, wherein the transcriptional regulatory element comprises a promoter element and/or an intron element.

Clause 29. The pharmaceutical composition of any one of clauses 23-28, wherein the rAAV genome further comprises a polyadenylation sequence.

Clause 30. The pharmaceutical composition of clause 29, wherein the polyadenylation sequence is 3′ to the transgene.

Clause 31. The pharmaceutical composition of any one of clauses 23-30, wherein the rAAV genome comprises a nucleotide sequence that is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 50, 51, 52, 53, or 54.

Clause 32. The pharmaceutical composition of any one of clauses 23-31, wherein the rAAV genome further comprises a 5′ inverted terminal repeat (5′ ITR) nucleotide sequence 5′ of the transgene, and a 3′ inverted terminal repeat (3′ ITR) nucleotide sequence 3′ of the transgene.

Clause 33. The pharmaceutical composition of clause 32, wherein the 5′ ITR nucleotide sequence is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 39, 41, or 42, and/or the 3′ ITR nucleotide sequence is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 40, 43, or 44.

Clause 34. The pharmaceutical composition of any one of clauses 23-33, wherein the rAAV genome comprises a nucleotide sequence that is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 55, 56, 57, 58, or 59.

Clause 35. The pharmaceutical composition of any one of clauses 23-34, wherein the rAAV comprises an AAV capsid comprising an AAV capsid protein.

Clause 36. The pharmaceutical composition of clause 35, wherein the AAV capsid protein is selected from the group consisting of AAV1, AAV2, AAV3, AAV4, AAV5, AAV6, AAV7, AAV8, AAV9, AAV-DJ, AAV-LK03, NP59, VOY101, VOY201, VOY701, VOY801, VOY1101, AAVPHP.N, AAVPHP.A, AAVPHP.B, PHP.B2, PHP.B3, G2A3, G2B4, G2B5, and PuP.S.

Clause 37. The pharmaceutical composition of clause 35 or 36, wherein the AAV capsid protein comprises an amino acid sequence that is at least 85% identical to the amino acid sequence of amino acids 203-736 of SEQ ID NO: 1, 2, 3, 4, 5, 6, 7, 8, 9, 10, 11, 12, 13, 15, 16, or 17.

Clause 38. The pharmaceutical composition of clause 37, wherein: the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

Clause 39. The pharmaceutical composition of clause 38, wherein:

(a) the amino acid in the capsid protein corresponding to amino acid 626 of SEQ ID NO: 16 is G, and the amino acid in the capsid protein corresponding to amino acid 718 of SEQ ID NO: 16 is G;
(b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M;
(c) the amino acid in the capsid protein corresponding to amino acid 505 of SEQ ID NO: 16 is R, and the amino acid in the capsid protein corresponding to amino acid 687 of SEQ ID NO: 16 is R;
(d) the amino acid in the capsid protein corresponding to amino acid 346 of SEQ ID NO: 16 is A, and the amino acid in the capsid protein corresponding to amino acid 505 of SEQ ID NO: 16 is R; or
(e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

Clause 40. The pharmaceutical composition of clause 37, wherein the AAV capsid protein comprises the amino acid sequence of amino acids 203-736 of SEQ ID NO: 1, 2, 3, 4, 5, 6, 7, 8, 9, 10, 11, 12, 13, 15, 16, or 17.

Clause 41. The pharmaceutical composition of any one of clauses 35-40, wherein the AAV capsid protein comprises an amino acid sequence that is at least 85% identical to the amino acid sequence of amino acids 138-736 of SEQ ID NO: 1, 2, 3, 4, 5, 6, 7, 8, 9, 10, 11, 12, 13, 15, 16, or 17.

Clause 42. The pharmaceutical composition of clause 41, wherein: the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

Clause 43. The pharmaceutical composition of clause 42, wherein:

(a) the amino acid in the capsid protein corresponding to amino acid 626 of SEQ ID NO: 16 is G, and the amino acid in the capsid protein corresponding to amino acid 718 of SEQ ID NO: 16 is G;
(b) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M;
(c) the amino acid in the capsid protein corresponding to amino acid 505 of SEQ ID NO: 16 is R, and the amino acid in the capsid protein corresponding to amino acid 687 of SEQ ID NO: 16 is R;
(d) the amino acid in the capsid protein corresponding to amino acid 346 of SEQ ID NO: 16 is A, and the amino acid in the capsid protein corresponding to amino acid 505 of SEQ ID NO: 16 is R; or
(e) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

Clause 44. The pharmaceutical composition of clause 41, wherein the AAV capsid protein comprises the amino acid sequence of amino acids 138-736 of SEQ ID NO: 1, 2, 3, 4, 5, 6, 7, 9, 10, 11, 12, 13, 15, 16, or 17.

Clause 45. The pharmaceutical composition of any one of clauses 35-44, wherein the AAV capsid protein comprises an amino acid sequence that is at least 85% identical to the amino acid sequence of amino acids 1-736 of SEQ ID NO: 1, 2, 3, 4, 5, 6, 7, 8, 9, 10, 11, 12, 13, 15, 16, or 17.

Clause 46. The pharmaceutical composition of clause 45, wherein: the amino acid in the capsid protein corresponding to amino acid 2 of SEQ ID NO: 16 is T; the amino acid in the capsid protein corresponding to amino acid 65 of SEQ ID NO: 16 is I; the amino acid in the capsid protein corresponding to amino acid 68 of SEQ ID NO: 16 is V; the amino acid in the capsid protein corresponding to amino acid 77 of SEQ ID NO: 16 is R; the amino acid in the capsid protein corresponding to amino acid 119 of SEQ ID NO: 16 is L; the amino acid in the capsid protein corresponding to amino acid 151 of SEQ ID NO: 16 is R; the amino acid in the capsid protein corresponding to amino acid 160 of SEQ ID NO: 16 is D; the amino acid in the capsid protein corresponding to amino acid 206 of SEQ ID NO: 16 is C; the amino acid in the capsid protein corresponding to amino acid 296 of SEQ ID NO: 16 is H; the amino acid in the capsid protein corresponding to amino acid 312 of SEQ ID NO: 16 is Q; the amino acid in the capsid protein corresponding to amino acid 346 of SEQ ID NO: 16 is A; the amino acid in the capsid protein corresponding to amino acid 464 of SEQ ID NO: 16 is N; the amino acid in the capsid protein corresponding to amino acid 468 of SEQ ID NO: 16 is S; the amino acid in the capsid protein corresponding to amino acid 501 of SEQ ID NO: 16 is I; the amino acid in the capsid protein corresponding to amino acid 505 of SEQ ID NO: 16 is R; the amino acid in the capsid protein corresponding to amino acid 590 of SEQ ID NO: 16 is R; the amino acid in the capsid protein corresponding to amino acid 626 of SEQ ID NO: 16 is G or Y; the amino acid in the capsid protein corresponding to amino acid 681 of SEQ ID NO: 16 is M; the amino acid in the capsid protein corresponding to amino acid 687 of SEQ ID NO: 16 is R; the amino acid in the capsid protein corresponding to amino acid 690 of SEQ ID NO: 16 is K; the amino acid in the capsid protein corresponding to amino acid 706 of SEQ ID NO: 16 is C; or, the amino acid in the capsid protein corresponding to amino acid 718 of SEQ ID NO: 16 is G.

Clause 47. The pharmaceutical composition of clause 46, wherein:

(a) the amino acid in the capsid protein corresponding to amino acid 2 of SEQ ID NO: 16 is T, and the amino acid in the capsid protein corresponding to amino acid 312 of SEQ ID NO: 16 is Q;
(b) the amino acid in the capsid protein corresponding to amino acid 65 of SEQ ID NO: 16 is I, and the amino acid in the capsid protein corresponding to amino acid 626 of SEQ ID NO: 16 is Y;
(c) the amino acid in the capsid protein corresponding to amino acid 77 of SEQ ID NO: 16 is R, and the amino acid in the capsid protein corresponding to amino acid 690 of SEQ ID NO: 16 is K;
(d) the amino acid in the capsid protein corresponding to amino acid 119 of SEQ ID NO: 16 is L, and the amino acid in the capsid protein corresponding to amino acid 468 of SEQ ID NO: 16 is S;
(e) the amino acid in the capsid protein corresponding to amino acid 626 of SEQ ID NO: 16 is G, and the amino acid in the capsid protein corresponding to amino acid 718 of SEQ ID NO: 16 is G;
(f) the amino acid in the capsid protein corresponding to amino acid 296 of SEQ ID NO: 16 is H, the amino acid in the capsid protein corresponding to amino acid 464 of SEQ ID NO: 16 is N, the amino acid in the capsid protein corresponding to amino acid 505 of SEQ ID NO: 16 is R, and the amino acid in the capsid protein corresponding to amino acid 681 of SEQ ID NO: 16 is M;
(g) the amino acid in the capsid protein corresponding to amino acid 505 of SEQ ID NO: 16 is R, and the amino acid in the capsid protein corresponding to amino acid 687 of SEQ ID NO: 16 is R;
(h) the amino acid in the capsid protein corresponding to amino acid 346 of SEQ ID NO: 16 is A, and the amino acid in the capsid protein corresponding to amino acid 505 of SEQ ID NO: 16 is R; or
(i) the amino acid in the capsid protein corresponding to amino acid 501 of SEQ ID NO: 16 is I, the amino acid in the capsid protein corresponding to amino acid 505 of SEQ ID NO: 16 is R, and the amino acid in the capsid protein corresponding to amino acid 706 of SEQ ID NO: 16 is C.

Clause 48. The pharmaceutical composition of clause 45, wherein the AAV capsid protein comprises the amino acid sequence of amino acids 1-736 of SEQ ID NO: 1, 2, 3, 4, 5, 6, 7, 8, 9, 10, 11, 12, 13, 15, 16, or 17.

Clause 49. A method of transducing a target cell in a subject, comprising administering to the subject a therapeutically effective amount of the pharmaceutical composition of any one of clauses 1-46 under conditions whereby the target cell is transduced.

Clause 50. A method of expressing a transgene in a target cell in a subject, comprising administering to the subject a therapeutically effective amount of the pharmaceutical composition of any one of clauses 1-46 under conditions whereby the target cell is transduced and the transgene is expressed.

Clause 51. The method of clause 49 or 50, wherein the target cell is a cell of the blood, liver, heart, joint tissue, muscle, brain, kidney, or lung.

Clause 52. The method of clause 49 or 50, wherein the target cell is a cell of the central nervous system, or the peripheral nervous system.

Clause 53. A method of treating or preventing a disease in a subject in need thereof, comprising administering to the subject a therapeutically effective amount of the pharmaceutical composition of any one of clauses 1-48.

Clause 54. The method of any one of clauses 49-53, wherein the formulation is administered to the subject intravenously, intraperitoneally, subcutaneously, intramuscularly, intrathecally, intracerebroventricularly, intradermally, or directly into the central nervous system of the subject.

Clause 55. The method of any one of clauses 49-54, wherein the subject is a human subject.

1. A pharmaceutical composition comprising:
(a) an adeno-associated virus (AAV);
(b) histidine;
(c) trehalose; and
(d) greater than about 150 mM sodium chloride.

2. The pharmaceutical composition of claim 1, comprising about 5 mM to about 50 mM histidine.

3. (canceled)

4. The pharmaceutical composition of claim 1, comprising about 1% (w/v %) to about 10% (w/v %) trehalose.

5. (canceled)

6. (canceled)

7. (canceled)

8. The pharmaceutical composition of claim 1, comprising no more than about 200 mM sodium chloride.

9. (canceled)

10. (canceled)

11. The pharmaceutical composition of claim 1, further comprising about 0.01% (w/v %) to about 0.05% (w/v %) Poloxamer 188.

12. (canceled)

13. The pharmaceutical composition of claim 1, comprising:
(a) an adeno-associated virus (AAV);
(b) about 20 mM histidine;
(c) about 3% (w/v %) trehalose;
(d) about 0.03% (w/v %) Poloxamer 188; and
(e) about 175 mM sodium chloride.

14. The pharmaceutical composition of claim 1, wherein the pH of the pharmaceutical composition is from about 6 to about 8.

15. The pharmaceutical composition of claim 1, wherein the pH of the pharmaceutical composition is from about 6.3 to 8.3.

16. (canceled)

17. (canceled)

18. The pharmaceutical composition of claim 1, comprising about 1e13 vg/mL to about 6e15 vg/mL of the AAV.

19. (canceled)

20. (canceled)

21. (canceled)

22. (canceled)

23. (canceled)

24. (canceled)

25. The pharmaceutical composition of claim 1, wherein the AAV is a recombinant AAV (rAAV) comprising an rAAV genome comprising a transgene.

26. (canceled)

27. (canceled)

28. The pharmaceutical composition of claim 25, wherein the transgene encodes a protein selected from the group consisting of glucose-6-phosphatase (G6Pase) and frataxin (FXN).

29. (canceled)

30. (canceled)

31. (canceled)

32. (canceled)

33. The pharmaceutical composition of claim 25, wherein the rAAV genome further comprises a 5′ inverted terminal repeat (5′ ITR) nucleotide sequence 5′ of the transgene, and a 3′ inverted terminal repeat (3′ ITR) nucleotide sequence 3′ of the transgene.

34. The pharmaceutical composition of claim 33, wherein the 5′ ITR nucleotide sequence is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 39, 41, or 42, and/or the 3′ ITR nucleotide sequence is at least 85%, 86%, 87%, 88%, 89%, 90%, 91%, 92%, 93%, 94%, 95%, 96%, 97%, 98%, 99%, or 100% identical to the nucleotide sequence set forth in SEQ ID NO: 40, 43, or 44.

35. The pharmaceutical composition of claim 25, wherein the rAAV comprises an AAV capsid comprising an AAV capsid protein.

36. The pharmaceutical composition of claim 35, wherein the AAV capsid protein is selected from the group consisting of AAV1, AAV2, AAV3, AAV4, AAV5, AAV6, AAV7, AAV8, AAV9, AAV10, AAV11, AAV12, AAV13, AAV-DJ, AAV-LK03, NP59, VOY101, VOY201, VOY701, VOY801, VOY1101, AAVPHP.N, AAVPHP.A, AAVPHP.B, PHP.B2, PHP.B3, G2A3, G2B4, G2B5, PHP.S, AAVRh32.33, AAVrh74, and AAVrh10.

37. A method of transducing a target cell in a subject, comprising administering to the subject a therapeutically effective amount of the pharmaceutical composition of claim 1 under conditions whereby the target cell is transduced.

38. A method of expressing a transgene in a target cell in a subject, comprising administering to the subject a therapeutically effective amount of the pharmaceutical composition of claim 1 under conditions whereby the target cell is transduced and the transgene is expressed.

39. (canceled)

40. (canceled)

41. A method of treating or preventing a disease in a subject in need thereof, comprising administering to the subject a therapeutically effective amount of the pharmaceutical composition of claim 1.

42. (canceled)

43. The method of claim 37, wherein the subject is a human subject.

44. A method for the preparation of a pharmaceutical composition of claim 1, wherein the method comprises the steps of mixing:
(a) an adeno-associated virus (AAV);
(b) histidine;
(c) trehalose; and
(d) greater than about 150 mM sodium chloride.

45. (canceled)

46. The method of claim 44, wherein the method further comprises the step of storing the pharmaceutical composition at a temperature from about −80° C. to about 25° C.

47. (canceled)

48. (canceled)

49. (canceled)

50. (canceled)
